(19) United States
(12) Patent Application Publication
(10) Pub. No.: US 2026/0347383 A1
(43) Pub. Date: Nov. 5, 2026

(54) CRYSTALLINE FORMS OF SIALIC ACID DERIVATIVES AND METHODS FOR THEIR USE

(71) Applicant: Ultragenyx Pharmaceutical Inc., Novato, CA (US)

(72) Inventors: Jean Carol BAUM, Novato, CA (US); Dong YAN, Novato, CA (US); Yen Thi Hong CU, Novato, CA (US)

(21) Appl. No.: 19/155,478

(22) PCT Filed: Feb. 16, 2024
(86) PCT No.: PCT/US2024/016157

§ 371 Date: Aug. 11, 2025

Related U.S. Application Data
(60) Provisional application No. 63/485,872, filed on Feb. 17, 2023.

(51) Int. Cl. C07H 13/06

(52) U.S. Cl. CPC C07H 13/06

(57) ABSTRACT

The present disclosure relates to solid forms of (1R,4R,5R,6R,7S)-6-acetamido-4-((R)-1,2-dihydroxyethyl)-7-hydroxy-2-oxo-3,9-dioxabicyclo[3.3.1]nonan-1-yl palmitate (Compound I) or a pharmaceutically acceptable salt and/or solvate thereof, pharmaceutical compositions comprising the solid forms, and therapeutic uses thereof. In particular, the present disclosure relates to solid forms of Compound I or a pharmaceutically acceptable salt and/or solvate thereof, which are useful for treating sialic acid deficiency.

This application claims the benefit of U.S. Provisional Application No. 63/485,872, filed Feb. 17, 2023, the disclosure of which is incorporated by reference herein in its entirety.

### FIELD OF THE INVENTION

The present disclosure relates to solid forms of Compound I or a pharmaceutically acceptable salt and/or solvate thereof, pharmaceutical compositions comprising the solid forms, and therapeutic uses thereof. In particular, the present disclosure relates to solid forms of Compound I or a pharmaceutically acceptable salt and/or solvate thereof, which are useful for treating sialic acid deficiency.

### BACKGROUND OF THE INVENTION

Sialic acid is a sugar that contains a net negative charge and is typically found on terminating branches of N-glycans, O-glycans, and glycosphingolipids (gangliosides) (and occasionally capping side chains of GPI anchors). The sialic acid modification of cell surface molecules is crucial for many biological phenomena including protein structure and stability, regulation of cell adhesion, and signal transduction. Thus, sialic acid abnormalities, such as sialic acid deficiency, can cause severe health problems. Sialic acid deficiency disorders, such as GNE myopathy (also known as hereditary inclusion body myopathy (HIBM), Nonaka myopathy, Nonaka distal myopathy, distal myopathy with rimmed vacuoles (DMRV), inclusion body myopathy 2 (IBM2), and quadriceps-sparing myopathy), are clinical diseases resulting from a reduction in sialic acid production.

Sialic acid production is dysregulated or reduced, for example, in individuals harboring mutations in the GNE gene, which encodes the bifunctional enzyme uridine diphospho-N-acetylglucosamine (UDP-GlcNAc) 2-epimerase/N-acetyl-mannosamine (ManNAc) kinase (GNE/MNK), which itself is critical in the biosynthesis of acetylneuraminic acid (Neu5Ac), also known as sialic acid. Huizing, Marjan, and Donna M. Krasnewich. Biochimica et Biophysica Acta (BBA)-Molecular Basis of Disease 1792.9: 881-887 (2009).

Because reduced production of sialic acid is the key reason mutations in the GNE gene causes disease, replacing a metabolite after the genetic block in the pathway could, in theory, alleviate symptoms of a sialic acid deficiency. Jay er al. Gene Reg. and Sys. Biology 3: 181-190 (2009). In practice, however, administering one or more compounds in the sialic acid biosynthetic pathway in vivo is a significant challenge. These compounds have extraordinarily rapid clearance rates and are excreted in the urine before they can be metabolized. Thus, prodrugs of sialic acids can be a useful strategy as described in WO 2013/063149, the contents of which are hereby incorporated by reference.

### SUMMARY OF THE INVENTION

The present disclosure relates to solid forms of Compound I:

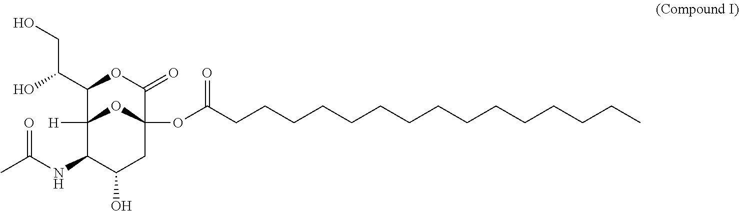 [SMILES: CCCCCCCCCCCCCCCC(=O)O[C@@]12C[C@H](O)[C@@H](NC(C)=O)[C@@H](O1)[C@@H]([C@H](O)CO)OC2=O]

or a pharmaceutically acceptable salt and/or solvate thereof, pharmaceutical compositions comprising the solid forms, and therapeutic uses thereof.

In embodiments, the present disclosure provides a solid form of Compound I or a pharmaceutically acceptable salt, solvate, or solvate salt thereof. In one embodiment, the crystalline form is solvated. In embodiments, the solvate is a hydrate. In one embodiment, the crystalline form is anhydrous or non-solvated. In one embodiment of the crystalline form, Compound I is not present as a pharmaceutically acceptable salt.

In one embodiment of the present disclosure, the crystalline form of Compound I or a pharmaceutically acceptable salt, solvate, or solvate salt thereof exhibits an X-ray powder diffraction (XRPD) pattern comprising peaks at about 16.6±0.2, about 18.0±0.2, and about 19.5±0.2 degrees two-theta. In one embodiment, the XRPD pattern further comprises peaks at about 3.9±0.2, about 11.5±0.2, about 15.3±0.2, and/or about 21.0±0.2 degrees two-theta. In one embodiment, the XRPD pattern further comprises a peak at about 11.7±0.2, about 14.2±0.2, and/or about 23.6±0.2 degrees two-theta. In embodiments, the crystalline form of Compound I exhibits an XRPD pattern substantially similar to FIG. 1A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 73 °C to about 77 °C. In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 150 °C to about 152 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 3.2% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.8±0.2, about 8.4±0.2, and about 19.9±0.2 degrees two-theta. In one embodiment, the XRPD pattern further comprises peaks at about 20.3±0.2, about 9.9±0.2, and/or about 13.9±0.2 degrees two-theta. In one embodiment, the XRPD pattern further comprises peaks at about 19.1±0.2, about 9.1±0.2, and/or about 12.3±0.2 degrees two-theta. In embodiments, the crystalline form of Compound I exhibits an XRPD pattern substantially similar to FIG. 2A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 77 °C to about 82 °C; and/or an endothermic peak having a peak maximum of from about 148 °C to about 152 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 6.8% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.9±0.2, about 8.5±0.2, and about 17.0±0.2 degrees two-theta. In one embodiment, the XRPD pattern further comprises peaks at about. In one embodiment, the XRPD pattern further comprises peaks at about 19.9±0.2, about 14.1±0.2, and/or about 22.9±0.2 degrees two-theta. In one embodiment, the XRPD pattern further comprises peaks at about. In one embodiment, the XRPD pattern further comprises peaks at about 10.6±0.2, about 19.3±0.2, and/or about 25.6±0.2 degrees two-theta. In embodiments, the crystalline form of Compound I exhibits an XRPD pattern substantially similar to FIG. 3A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 81 °C to about 85 °C; and/or an endothermic peak having a peak maximum of from about 148 °C to about 152 °C. In one embodiment of the present disclosure, the crystalline form of Compound I or a pharmaceutically acceptable salt, solvate, or solvate salt thereof exhibits a thermogravimetric analysis (TGA) thermogram that at exhibits weight percent loss of about 5.3% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.8±0.2, about 3.9±0.2, and about 8.2±0.2 degrees two-theta degrees two-theta. In one embodiment, the XRPD pattern further comprises peaks at about 15.4±0.2, about 11.5±0.2, and/or about 7.7±0.2 degrees two-theta. In one embodiment, the XRPD pattern further comprises peaks at about 19.2±0.2, about 16.4±0.2, and/or about 34.9±0.2 degrees two-theta. In embodiments, the crystalline form of Compound I exhibits an XRPD pattern substantially similar to FIG. 4.

In one embodiment of the present disclosure, the crystalline form of Compound I or a pharmaceutically acceptable salt, solvate, or solvate salt thereof exhibits an X-ray powder diffraction (XRPD) pattern comprising peaks at about 3.1±0.2, about 9.3±0.2, and about 18.6±0.2 degrees two-theta. In one embodiment, the XRPD pattern further comprises peaks at about 19.3±0.2, about 6.2±0.2, and/or about 21.8±0.2 degrees two-theta. In one embodiment, the XRPD pattern further comprises peaks at about 24.8±0.2, about 12.4±0.2, and/or about 15.0±0.2 degrees two-theta. In embodiments, the crystalline form of Compound I exhibits an XRPD pattern substantially similar to FIG. 5A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 34 °C to about 38 °C, an endothermic peak having a peak maximum of from about 76 °C to about 80 °C and/or an endothermic peak having a peak maximum of from about 148 °C to about 152 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 2.9% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.9±0.2, about 8.5±0.2, and about 20.3±0.2 degrees two-theta. In one embodiment, the XRPD pattern further comprises peaks at about 14.1±0.2, about 19.7±0.2, and/or about 10.0±0.2 degrees two-theta. In one embodiment, the XRPD pattern further comprises peaks at about 10.6±0.2, about 22.7±0.2, and/or about 16.9±0.2 degrees two-theta. In embodiments, the crystalline form of Compound I exhibits an XRPD pattern substantially similar to FIG. 6A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 81 °C to about 85 °C, and/or an endothermic peak having a peak maximum of from about 148 °C to about 152 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 23.2% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.8±0.2, about 8.3±0.2, and about 19.9±0.2 degrees two-theta degrees two-theta. In one embodiment, the XRPD pattern further comprises peaks at about 13.9±0.2, about 9.8±0.2, and/or about 10.5±0.2 degrees two-theta. In one embodiment, the XRPD pattern further comprises peaks at about 11.3±0.2, about 19.3±0.2, and/or about 24.9±0.2 degrees two-theta. In embodiments, the crystalline form of Compound I exhibits an XRPD pattern substantially similar to FIG. 7A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 90 °C to about 95 °C; and/or an endothermic peak having a peak maximum of from about 150 °C to about 154 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 7.7% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.8±0.2, about 8.1±0.2, and about 21.7±0.2 degrees two-theta. In one embodiment, the XRPD pattern further comprises peaks at about 10.8±0.2, about 16.3±0.2, and/or about 19.0±0.2 degrees two-theta. In one embodiment, the XRPD pattern further comprises peaks at about 5.4±0.2, about 19.8±0.2, and/or about 22.0±0.2 degrees two-theta. In embodiments, the crystalline form of Compound I exhibits an XRPD pattern substantially similar to FIG. 8A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 94 °C to about 98 °C; and/or an endothermic peak having a peak maximum of from about 149 °C to about 153 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 3.0% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.5±0.2, about 2.8±0.2, and about 8.4±0.2 degrees two-theta. In one embodiment, the XRPD pattern further comprises peaks at about 5.0±0.2, about 7.4±0.2, and/or about 16.9±0.2 degrees two-theta. In one embodiment, the XRPD pattern further comprises peaks at about 12.1±0.2, about 14.1±0.2, and/or about 19.7±0.2 degrees two-theta. In embodiments, the crystalline form of Compound I exhibits an XRPD pattern substantially similar to FIG. 9.

In one embodiment of the present disclosure, the crystalline form of Compound I or a pharmaceutically acceptable salt, solvate, or solvate salt thereof exhibits an X-ray powder diffraction (XRPD) pattern comprising peaks at about 2.8±0.2, about 8.2±0.2, and about 19.2±0.2 degrees two-theta. In one embodiment, the XRPD pattern further comprises peaks at about 11.0±0.2, about 11.3±0.2, and/or about 16.4±0.2 degrees two-theta. In one embodiment, the XRPD pattern further comprises peaks at about 5.5±0.2, and/or about 9.9±0.2 degrees two-theta. In embodiments, the crystalline form of Compound I exhibits an XRPD pattern substantially similar to FIG. 10A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 40 °C to about 50 °C; and/or an endothermic peak having a peak maximum of from about 150 °C to about 165 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 27.0% by thermogravimetric analysis (TGA).

In one embodiment of the present disclosure, the crystalline form of Compound I or a pharmaceutically acceptable salt, solvate, or solvate salt thereof exhibits an X-ray powder diffraction (XRPD) pattern comprising peaks at about 3.0±0.2, about 8.9±0.2, and about 19.1±0.2 degrees two-theta. In one embodiment, the XRPD pattern further comprises peaks at about 22.5±0.2 degrees two-theta. In embodiments, the crystalline form of Compound I exhibits an XRPD pattern substantially similar to FIG. 11A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 75 °C to about 80 °C and/or an endothermic peak having a peak maximum of from about 148 °C to about 153 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 1.8% by thermogravimetric analysis (TGA).

In embodiments, the crystalline form of Compound I exhibits an XRPD pattern substantially similar to FIG. 12A.

In one embodiment of the present disclosure, the crystalline form of Compound I or a pharmaceutically acceptable salt, solvate, or solvate salt thereof exhibits a differential scanning calorimetry (DSC) thermogram comprising an endothermic peak having a peak maximum of from about 148 °C to about 153 °C. In one embodiment of the present disclosure, the crystalline form of Compound I or a pharmaceutically acceptable salt, solvate, or solvate salt thereof exhibits a thermogravimetric analysis (TGA) thermogram that exhibits weight percent loss of about 0.4% by thermogravimetric analysis (TGA).

In embodiments, the present disclosure relates to an amorphous form of Compound I or a pharmaceutically acceptable salt, solvate, or solvate salt thereof. In one embodiment of the amorphous form, Compound I is not present as a pharmaceutically acceptable salt. In another embodiment, the amorphous form of Compound I or a pharmaceutically acceptable salt, solvate, or solvate salt thereof is in a pharmaceutical composition. In embodiments, the amorphous form of Compound I exhibits an XRPD pattern substantially similar to FIG. 13A.

In one embodiment of the present disclosure, the amorphous form of Compound I or a pharmaceutically acceptable salt, solvate, or solvate salt thereof exhibits a thermogravimetric analysis (TGA) thermogram that exhibits weight percent loss of about 0.6% by thermogravimetric analysis (TGA).

In embodiments, the present disclosure also relates to compositions comprising any one of the crystalline forms or the amorphous forms of Compound I or a pharmaceutically acceptable salt, solvate, or solvate salt thereof as disclosed herein, and a pharmaceutically acceptable carrier. In embodiments of the present disclosure, the composition is formulated as an oral dosage form. In embodiments, the solid oral dosage form comprises at least one tablet, at least one capsule, or granules. In embodiments, the oral dosage form is a liquid oral dosage form.

In one embodiment, any one of the compositions disclosed herein can further comprising an additional therapeutic agent. In one embodiment, any one of the compositions disclosed herein can further comprising one or more additional therapeutic agents.

In embodiments, the present disclosure also relates to methods for treating or preventing sialic acid deficiency comprising administering any one of the crystalline forms or the amorphous forms of Compound I or a pharmaceutically acceptable salt, solvate, or solvate salt thereof as disclosed herein.

In embodiments, the present disclosure also relates to methods for treating or preventing GNE myopathy comprising administering any one of the crystalline forms or the amorphous forms of Compound I or a pharmaceutically acceptable salt, solvate, or solvate salt thereof as disclosed herein.

### BRIEF DESCRIPTION OF THE DRAWINGS

FIG. 1A shows an X-ray powder diffraction (XRPD) pattern of a Form A of Compound 1 (monohydrate). FIG. 1B shows an overlay of a thermo-gravimetric analysis (TGA) thermogram and a differential scanning calorimetry (DSC) thermogram of a Form A of Compound 1 (monohydrate). FIG. 1C shows dynamic vapor sorption (DVS) plot of a Form A of Compound 1 (monohydrate).

FIG. 2A shows an XRPD pattern of a Form B of Compound 1. FIG. 2B shows an overlay of a TGA thermogram and a DSC thermogram of a Form B of Compound 1. FIG. 2C shows a DVS plot of a Form B of Compound 1.

FIG. 3A shows an XRPD pattern of a Form C of Compound 1. FIG. 3B shows an overlay of a TGA thermogram and a DSC thermogram of a Form C of Compound 1. FIG. 3C shows a DVS plot of a Form C of Compound 1.

FIG. 4 shows an XRPD pattern of a Form D of Compound 1.

FIG. 5A shows an XRPD pattern of a Form E of Compound 1. FIG. 5B shows an overlay of a TGA thermogram and a DSC thermogram of a Form E of Compound 1. FIG. 5C shows a DVS plot of a Form E of Compound 1.

FIG. 6A shows an XRPD pattern of a Form F of Compound 1. FIG. 6B shows an overlay of a TGA thermogram and a DSC thermogram of a Form F of Compound 1.

FIG. 7A shows an XRPD pattern of a Form G of Compound 1. FIG. 7B shows an overlay of a TGA thermogram and a DSC thermogram of a Form G of Compound 1.

FIG. 8A shows an XRPD pattern of a Form H of Compound 1. FIG. 8B shows an overlay of a TGA thermogram and a DSC thermogram of a Form H of Compound 1.

FIG. 9 shows an XRPD pattern of a Form I of Compound 1.

FIG. 10A shows an XRPD pattern of a Form J of Compound 1. FIG. 10B shows an overlay of a TGA thermogram and a DSC thermogram of a Form J of Compound 1.

FIG. 11A shows an XRPD pattern of a Form K of Compound 1. FIG. 11B shows an overlay of a TGA thermogram and a DSC thermogram of a Form K of Compound 1.

FIG. 12A shows an XRPD pattern of a Form L of Compound 1. FIG. 12B shows an overlay of a TGA thermogram and a DSC thermogram of a Form L of Compound 1.

FIG. 13A shows an XRPD pattern of an amorphous form of Compound 1 (anhydrous). FIG. 13B shows a TGA thermogram of an amorphous form of Compound 1 (anhydrous). FIG. 13C shows a DVS plot of an amorphous form of Compound 1 (anhydrous).

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### DEFINITIONS

While the following terms are believed to be well understood by one of ordinary skill in the art, the following definitions are set forth to facilitate explanation of the presently disclosed subject matter.

Compound 1 is (1R,4R,5R,6R,7S)-6-acetamido-4-((R)-1,2-dihydroxyethyl)-7-hydroxy-2-oxo-3,9-dioxabicyclo[3.3.1]nonan-1-yl palmitate having the structure shown below. Compound I is disclosed in WO 2013/063149, which is hereby incorporated by reference in its entirety.

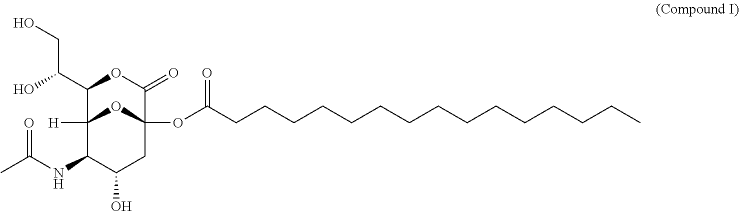 [SMILES: CCCCCCCCCCCCCCCC(=O)O[C@@]12C[C@H](O)[C@@H](NC(C)=O)[C@@H](O1)[C@@H]([C@H](O)CO)OC2=O]

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably. Unless otherwise noted, the term “about” when immediately preceding a numerical value means ±10% of the numerical value.

Throughout the present specification, numerical ranges are provided for certain quantities. It is to be understood that these ranges comprise endpoints and all subranges therein, including each integer in and between a disclosed range. Thus, the range “from 50 to 80” includes all possible ranges therein (e.g., 51-79, 52-78, 53-77, 54-76, 55-75, 60-70, etc.) as well as each individual integer from 50 to 80 (e.g., 50, 51, 52, 53, 54, etc.). Where ranges are provided in the form or fractions, percentages, decimals, and the like, such ranges likewise include all possible subranges therein and each individual fraction, percentage, decimal, etc. in and between the disclosed range. For example, the range “from 0.1 to 1.0” includes all possible ranges therein (e.g., 0.2 to 0.9, etc.) and each individual 1/10^{th} decimal from 0.1 to 1.0 (e.g., 0.1, 0.2, 0.3, 0.4, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of” the steps, elements, and/or reagents described in the claims.

The term “solvate” means a complex formed by solvation (the combination of solvent molecules with molecules or ions of the active agent of the present invention), or an aggregate that consists of a solute ion or molecule (the active agent of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form (solvate salt). The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g., ethyl acetate; may be embedded in the crystal grating. Preferred are pharmaceutically acceptable solvents.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof. In some embodiments, a “therapeutically effective amount” refers to the amount of any one of the solid forms or the compositions as disclosed herein that is effective to treat or prevent sialic acid deficiency, including GNE myopathy, in a subject in need thereof.

The term “subject” can refer to a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

In one embodiment, the crystalline forms are characterized by the interlattice plane intervals determined by an X-ray powder diffraction (XRPD) pattern. The spectrum of XRPD is typically represented by a diagram plotting the intensity of the peaks versus the location of the peaks, i.e., diffraction angle 2θ (two-theta) in degrees. The characteristic peaks of a given XRPD can be selected according to the peak locations and their relative intensity to conveniently distinguish this crystalline structure from others.

Those skilled in the art recognize that the measurements of the XRPD peak locations and/or intensity for a given crystalline form of the same compound will vary within a margin of error. The values of degree 2θ allow appropriate error margins. Typically, the error margins are represented by “±”. For example, the degree 2θ of about “3.8±0.2” denotes a range from about 3.6 to 4.0 degree 2θ. Depending on the sample preparation techniques, the calibration techniques applied to the instruments, human operational variation, and etc., those skilled in the art recognize that the appropriate error of margins for a XRPD can be about ±0.5; ±0.4; ±0.3; ±0.2; ±0.1; ±0.05; or less. Additional details of the methods and equipment used for the XRPD analysis are described in the Examples section.

In one embodiment, the crystalline forms are characterized by a differential scanning calorimetry (DSC). The DSC thermogram is typically expressed by a diagram plotting the normalized heat flow in units of Watts/gram (“W/g”) versus the measured sample temperature in degree Celsius. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others. Those skilled in the art recognize that the measurements of the DSC thermogram for a given crystalline form of the same compound will vary within a margin of error. The values of a single peak characteristic value, expressed in degree Celsius, allow appropriate error margins. Depending on the sample preparation techniques, the calibration techniques applied to the instruments, human operational variations, and etc., those skilled in the art recognize that the appropriate error of margins for a DSC thermogram characteristic value (°C) can be ±3; ±2.5; ±2.0; ±1.5; ±1.0; ±0.5; or less. Additional details of the methods and equipment used for the DSC thermogram analysis are described in the Examples section.

In one embodiment, the crystalline forms are characterized by a thermogravimetric analysis (TGA). The TGA thermogram is typically expressed by a diagram plotting the weight (%) versus the measured sample temperature in degrees Celsius. The TGA thermogram is usually evaluated to measure weight loss of the sample due to heating. Those skilled in the art recognize that the measurements of the TGA thermogram for a given crystalline form of the same compound will vary within a margin of error. Depending on the sample preparation techniques, the calibration techniques applied to the instruments, human operational variations, and etc., those skilled in the art recognize that the appropriate error of margins for a TGA thermogram characteristic value (%) can be ±3; ±2.5; ±2.0; ±1.5; ±1.0; ±0.5; or less. Additional details of the methods and equipment used for the DSC thermogram analysis are described in the Examples section.

In one embodiment, the crystalline forms are characterized by a dynamic vapor sorption (DVS) analysis. The DVS profile is typically expressed by a diagram plotting the sample relative humidity (RH) versus the change in mass (%). The DVS profile provides information on hygroscopicity of the crystalline form at different RH conditions. Additional details of the methods and equipment used for DVS are described in the Examples section.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, “substantially the same as” when used in connection with an XRPD pattern means that that one skilled in the art would be able to identify two solid forms being of the same crystalline form based on XRPD patterns. In some embodiments, “substantially the same as” when used in connection with an XRPD pattern means each peak of the XRPD pattern that is unique to a specific crystalline form differs from a respective peak of a reference compound by no more than ±0.2 degrees 2θ.

As used herein, “substantially the same as” when used in connection with an XRPD pattern means that that one skilled in the art would be able to identify two solid forms being of the same crystalline form based on XRPD patterns. In some embodiments, “substantially the same as” when used in connection with an XRPD pattern means each peak of the XRPD pattern that is unique to a specific crystalline form differs from a respective peak of a reference compound by no more than ±0.2 degrees 2θ.

As used herein, “substantially the same as” when used in connection with a DSC thermogram means that each peak of the DSC thermogram differs from a respective peak of a reference DSC thermogram by no more than ±3 °C.

As used herein, “substantially the same as” when used in connection with a TGA thermogram means that each peak of the TGA thermogram differs from a respective peak of a reference TG thermogram by no more than ±2%.

The present disclosure includes information that may be useful in understanding the present invention. It is not an admission that any of the information provided herein is prior art or relevant to the presently claimed inventions, or that any publication specifically or implicitly referenced is prior art.

### Solid Forms of Compound I

In one embodiment, the present disclosure relates to solid forms of Compound I or a pharmaceutically acceptable salt, solvate, or solvate salt thereof. In one embodiment, the solid form is for Compound I hydrate. In one embodiment, the solid form is for Compound I solvate. In one embodiment, the solid form is for Compound I solvate salt. In one embodiment, the solid form is for Compound I (not a salt, not a solvate, not a solvate salt). In one embodiment, the solid form is for a pharmaceutically acceptable salt of Compound I. In one embodiment the solid form is amorphous or crystalline form. In one embodiment the solid form is a polymorph.

In another embodiment, the amorphous form of Compound I or a pharmaceutically acceptable salt, solvate, or solvate salt thereof is in a pharmaceutical composition. In a specific embodiment, the pharmaceutical composition comprises Compound I in a solid dispersion.

In one embodiment, the solid form of Compound I is crystalline Form A. In one embodiment, the solid form of Compound I is Form A, Form B, Form C, Form D, Form E, Form F, Form G, Form H, Form I, Form J, Form K, or Form L. In one embodiment, the solid form of Compound I is an amorphous form.

In some embodiments, the solid forms of Compound I is described by its XRPD peaks.

In one embodiment, the present disclosure relates to an isolated solid form of Compound I or a pharmaceutically acceptable salt, solvate, or solvate salt thereof. In one embodiment, the isolated solid form is an isolated crystalline form of Compound I or a pharmaceutically acceptable salt, solvate, or solvate salt thereof. In one embodiment, the isolated solid form is an isolated crystalline Form A of Compound I. In one embodiment, the isolated solid form is an isolated amorphous form of Compound I or a pharmaceutically acceptable salt, solvate, or solvate salt thereof. In one embodiment, the isolated solid form is an isolated amorphous form of Compound I.

In one embodiment, the solid form of Compound I or a pharmaceutically acceptable salt, solvate, or solvate salt thereof has a purity of at least about 75%, at least about 80%, at least about 85%, at least about 90%, at least about 95%, at least about 97%, at least about 98%, or at least about 99%. In one embodiment, the solid form of Compound I or a pharmaceutically acceptable salt, solvate, or solvate salt thereof has a purity of at least about 75%, at least about 80%, at least about 85%, at least about 90%, at least about 95%, at least about 97%, at least about 98%, or at least about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 90% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 95% to about 99.9% with respect to one specific solid form of Compound I or a pharmaceutically acceptable salt, solvate, or solvate salt thereof.

In one embodiment, the solid form of Compound I or a pharmaceutically acceptable salt, solvate, or solvate salt thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the solid form of Compound I or a pharmaceutically acceptable salt, solvate, or solvate salt thereof has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90% with respect to one specific solid form of Compound I or a pharmaceutically acceptable salt, solvate, or solvate salt thereof.

In one embodiment, the solid form of Compound I or a pharmaceutically acceptable salt, solvate, or solvate salt thereof has a purity of about 75% to about 99%. In one embodiment, the solid form of Compound I or a pharmaceutically acceptable salt, solvate, or solvate salt thereof has a purity of about 80% to about 99%. In one embodiment, the solid form of Compound I or a pharmaceutically acceptable salt, solvate, or solvate salt thereof has a purity of about 85% to about 99%. In one embodiment, the solid form of Compound I or a pharmaceutically acceptable salt, solvate, or solvate salt thereof has a purity of about 90% to about 99%. In one embodiment, the solid form of Compound I or a pharmaceutically acceptable salt, solvate, or solvate salt thereof has a purity of about 95% to about 99%. In one embodiment, the solid form of Compound I or a pharmaceutically acceptable salt, solvate, or solvate salt thereof has a purity of about 95% to about 99.9%.

In one embodiment, the solid form of Compound I or a pharmaceutically acceptable salt, solvate, or solvate salt thereof has a purity of about 75% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 80% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 85% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 90% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 95% to about 99% with respect to one specific solid form of Compound I or a pharmaceutically acceptable salt, solvate, or solvate salt thereof. In one embodiment, the solid form of Compound I or a pharmaceutically acceptable salt, solvate, or solvate salt thereof has a purity of about 95% to about 99.9% with respect to one specific solid form of Compound I or a pharmaceutically acceptable salt, solvate, or solvate salt thereof.

In one embodiment, the specific solid form of Compound I or a pharmaceutically acceptable salt, solvate, or solvate salt thereof with high purity is crystalline Form A of Compound I. In one embodiment, the specific solid form of Compound I or a pharmaceutically acceptable salt, solvate, or solvate salt thereof with high purity is an amorphous form of Compound I.

In one embodiment, the present disclosure relates to solid forms of Compound I or a pharmaceutically acceptable salt, solvate, or solvate salt thereof, wherein the solid form comprises one or more solid forms of Compound I or a pharmaceutically acceptable salt, solvate, or solvate salt thereof. In one embodiment, the solid form of the present disclosure comprises one or more forms selected from the group consisting of: Form A, Form B, Form C, Form D, Form E, Form F, Form G, Form H, Form I, Form J, Form K, Form L, or amorphous form of Compound I.

### Form A of Compound I

In one embodiment, the present disclosure relates to a Form A of Compound I or a pharmaceutically acceptable salt, solvate, or solvate salt thereof. In some embodiments, Form A is a monohydrate of Compound I. In one embodiment, Form A is crystalline.

In some embodiments, a monohydrate of Compound I exhibits an X-ray powder diffraction (XRPD) pattern comprising a peak at about 3.9±0.2, about 16.6±0.2, and about 19.5±0.2 degrees two-theta. In some embodiments, the monohydrate of Compound I exhibits an XRPD pattern further comprising a peak at about 11.5±0.2, about 15.3±0.2, about 18.0±0.2, and/or about 21.0±0.2 degrees two-theta. In some embodiments, the monohydrate of Compound I exhibits an XRPD pattern further comprising a peak at about 11.7±0.2, about 14.2±0.2, and/or about 23.6±0.2 degrees two-theta.

In some embodiments, a monohydrate of Compound I exhibits an XRPD pattern comprising a peak at about 18.0±0.2 degrees two-theta. In some embodiments, the monohydrate of Compound I exhibits an XRPD pattern further comprising a peak at about 16.6±0.2, and about 19.5±0.2 degrees two-theta. In some embodiments, the monohydrate of Compound I exhibits an XRPD pattern further comprising a peak at about 15.3±0.2 and about 21.0±0.2 degrees two-theta.

In some embodiments, a monohydrate of Compound I exhibits an XRPD pattern comprising a peak at about 16.6±0.2, about 18.0±0.2, and about 19.5±0.2 degrees two-theta.

In some embodiments, a monohydrate of Compound I exhibits an XRPD pattern comprising a peak at about 15.3±0.2, about 16.6±0.2, about 18.0±0.2, about 19.5±0.2, and about 21.0±0.2 degrees two-theta.

In some embodiments, Form A of Compound I, or a pharmaceutically acceptable salt, solvate, or solvate salt thereof, exhibits an X-ray powder diffraction (XRPD) pattern comprising a peak at about 3.9±0.2, about 16.6±0.2, and about 19.5±0.2 degrees two-theta. In some embodiments, Form A exhibits an XRPD pattern further comprising a peak at about 11.5±0.2, about 15.3±0.2, about 18.0±0.2, and/or about 21.0±0.2 degrees two-theta. In some embodiments, Form A exhibits an XRPD pattern further comprising a peak at about 11.7±0.2, about 14.2±0.2, and/or about 23.6±0.2 degrees two-theta.

In some embodiments, Form A exhibits an XRPD pattern comprising a peak at about 18.0±0.2 degrees two-theta. In some embodiments, Form A exhibits an XRPD pattern further comprising a peak at about 16.6±0.2, and about 19.5±0.2 degrees two-theta. In some embodiments, Form A exhibits an XRPD pattern further comprising a peak at about 15.3±0.2 and about 21.0±0.2 degrees two-theta.

In some embodiments, Form A exhibits an XRPD pattern comprising a peak at about 16.6±0.2, about 18.0±0.2, and about 19.5±0.2 degrees two-theta.

In some embodiments, Form A exhibits an XRPD pattern comprising a peak at about 15.3±0.2, about 16.6±0.2, about 18.0±0.2, about 19.5±0.2, and about 21.0±0.2 degrees two-theta.

In some embodiments, Form A exhibits an XRPD pattern comprising 1 peak, 2 peaks, 3 peaks, 4 peaks, 5 peaks, 6 peaks, 7 peaks, 8 peaks, 9 peaks, or 10 peaks of Table A. In some embodiments, Form A exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 3.9±0.2, about 11.5±0.2, 11.7±0.2, about 14.2±0.2, about 15.3±0.2, about 16.6±0.2, about 18.0±0.2, about 19.5±0.2, about 21.0±0.2, and about 23.6±0.2 degrees two-theta.

In some embodiments, Form A exhibits an XRPD pattern comprising a peak from Table A having an intensity (I %) of greater than 8%. In some embodiments, Form A exhibits an XRPD pattern comprising a peak from Table A having an intensity (I %) of greater than 7%. In some embodiments, Form A exhibits an XRPD pattern comprising a peak from Table A having an intensity (I %) of greater than 6%. In some embodiments, Form A exhibits an XRPD pattern comprising a peak from Table A having an intensity (I %) of greater than 5%. In some embodiments, Form A exhibits an XRPD pattern comprising a peak from Table A having an intensity (I %) of greater than 4%.

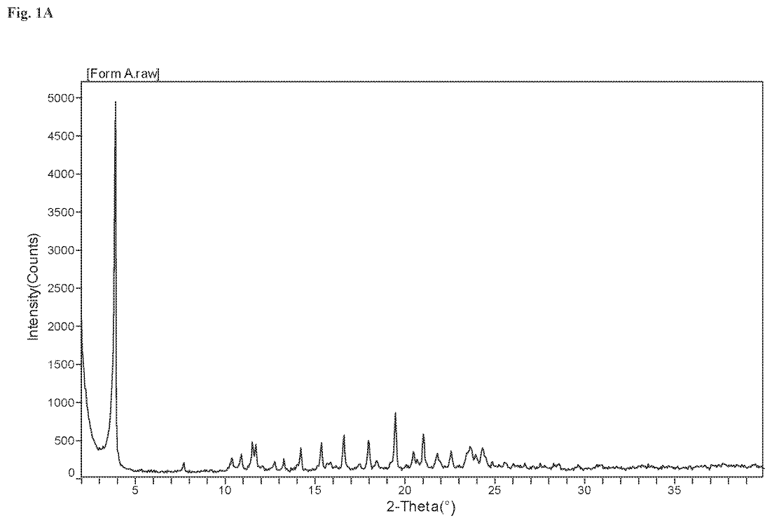

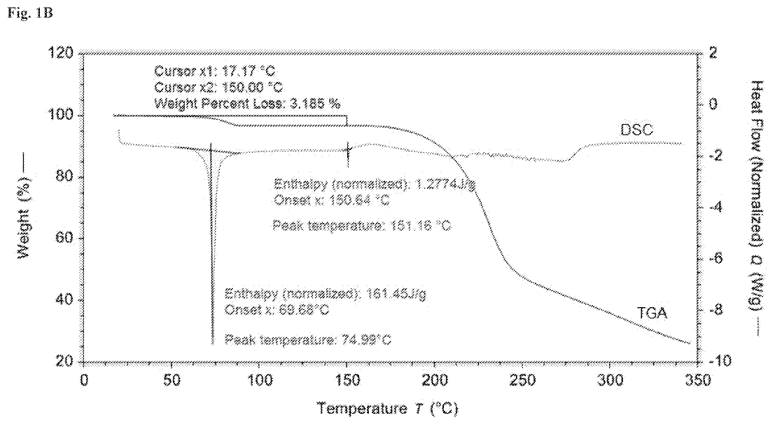

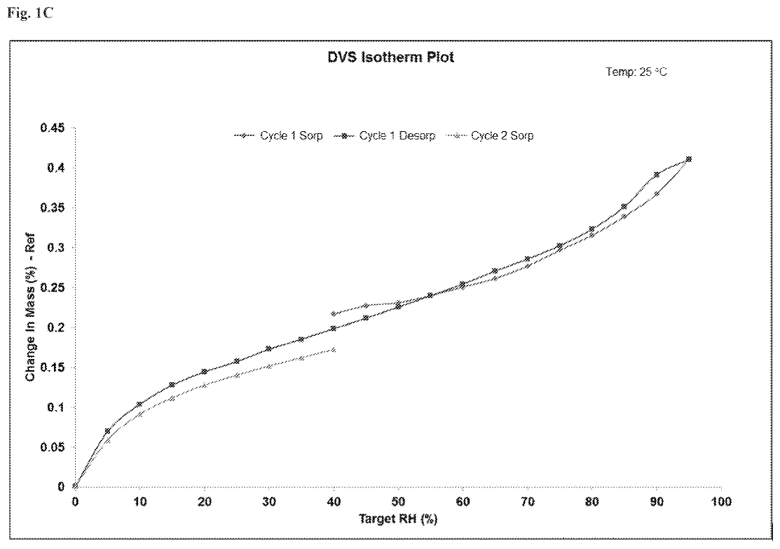

In some embodiments, Form A exhibits an XRPD pattern that is substantially the same as that depicted in FIG. 1A.

TABLE A
XRPD Table of Form A of Compound I

| # | 2-Theta | d(A) | I % |
|---|---|---|---|
| 1 | 3.873 | 22.7942 | 100 0 |
| 2 | 7.686 | 11.4924 | 2.5 |
| 3 | 10.355 | 8.5355 | 3.2 |
| 4 | 10.886 | 8.1203 | 3.9 |
| 5 | 11.522 | 7.6737 | 7.2 |
| 6 | 11.691 | 7.5634 | 6.5 |
| 7 | 12.742 | 6.416 | 2.1 |
| 8 | 13.258 | 6.6726 | 2.9 |
| 9 | 14.198 | 6.2327 | 6.0 |
| 10 | 15.346 | 5.7690 | 7.1 |
| 11 | 15.841 | 5.5899 | 1.6 |
| 12 | 16.600 | 5.3360 | 8.9 |
| 13 | 17.978 | 4.9301 | 7.4 |
| 14 | 18.434 | 4.8069 | 0.8 |
| 15 | 19.467 | 4.5561 | 14.9 |
| 16 | 20.476 | 4.3338 | 4.2 |
| 17 | 21.029 | 4.2211 | 8.7 |
| 18 | 21.810 | 4.0716 | 3.4 |
| 19 | 22.566 | 3.9369 | 4.2 |
| 20 | 23.625 | 3.7627 | 5.0 |
| 21 | 24.323 | 3.6564 | 4.4 |
| 22 | 24.856 | 3.5791 | 1.0 |
| 23 | 25.568 | 3.4811 | 1.0 |
| 24 | 26.036 | 3.4196 | 1.0 |
| 25 | 26.666 | 3.3402 | 1.1 |

In some embodiments, Form A has a melting range from about 130 °C to about 165 °C.

In some embodiments, Form A exhibits a differential scanning calorimetry (DSC) thermogram comprising an endothermic peak having a peak maximum of from about 70 °C to about 80 °C. In some embodiments, Form A exhibits a DSC thermogram comprising an endothermic peak having a peak maximum of from about 73 °C to about 77 °C. In some embodiments, Form A exhibits a DSC thermogram comprising an endothermic peak having a peak maximum of about 73 °C. In some embodiments, Form A exhibits a DSC thermogram comprising an endothermic peak having a peak maximum of about 75 °C. In some embodiments, Form A exhibits a DSC thermogram comprising an endothermic peak that onsets at about 70 °C. In some embodiments, Form A exhibits a DSC thermogram comprising an endothermic peak that onsets at about 72 °C.

In some embodiments, Form A exhibits a differential scanning calorimetry (DSC) thermogram comprising an endothermic peak having a peak maximum of from about 148 °C to about 153 °C. In some embodiments, Form A exhibits a differential scanning calorimetry (DSC) thermogram comprising an endothermic peak having a peak maximum of from about 150 °C to about 153 °C. In some embodiments, Form A exhibits a DSC thermogram comprising an endothermic peak having a peak maximum of from about 150 °C to about 152 °C. In some embodiments, Form A exhibits a DSC thermogram comprising an endothermic peak having a peak maximum of about 151 °C. In some embodiments, Form A exhibits a DSC thermogram comprising an endothermic peak that onsets at about 150 °C.

In some embodiments, Form A exhibits weight percent loss of about 3.2% by thermogravimetric analysis (TGA). In some embodiments, Form A exhibits weight percent loss of about 3.2% by TGA from room temperature to about 150 °C.

In some embodiments, Form A exhibits a DSC thermogram that is substantially the same as that depicted in FIG. 1B. In some embodiments, Form A exhibits a TGA thermogram that is substantially the same as that depicted in FIG. 1B.

In some embodiments, the water content of Form A is about 3.5% by volumetric Karl Fischer titration.

In some embodiments, Form A water uptake from 40% RH to 80% RH at 25 °C is less than about 1% by weight. In some embodiments, Form A water uptake from 40% RH to 80% RH at 25 °C is less than about 0.5% by weight. Form A water uptake from 40% RH to 80% RH at 25 °C is less than about 0.2% by weight. In some embodiments, Form A water uptake from 40% RH to 80% RH at 25 °C is less than about 0.1% by weight. In some embodiments, Form A is non-hygroscopic. In some embodiments, water uptake is measured by dynamic vapor sorption (DVS).

In some embodiments, Form A exhibits a DVS spectrum that is substantially the same as that depicted in FIG. 1C.

In some embodiments, Form A is purified. In some embodiments, Form A is isolated. In some embodiments, Form A is purified and isolated.

In some embodiments, Form A is at least about 90% pure by weight. In some embodiments, Form A is at least about 95% pure by weight. In some embodiments, Form A is at least about 95% pure by weight. In some embodiments, Form A is at least about 99% pure by weight. In some embodiments, Form A is at least about 90%, about 91%, about 92%, about 93%, about 94%, about 95%, about 96%, about 97%, about 98%, or about 99% pure by weight. In some embodiments, Form A is about 90% to about 99% pure by weight. In some embodiments, Form A is about 90% to about 99.9% pure by weight.

In some embodiments, Form A obtained according to Example 2 is at least about 90% pure by weight. In some embodiments, Form A obtained according to Example 2 is at least about 95% pure by weight. In some embodiments, Form A obtained according to Example 2 is at least about 95% pure by weight. In some embodiments, Form A obtained according to Example 2 is at least about 99% pure by weight. In some embodiments, Form A obtained according to Example 2 is about 95% to about 99% pure by weight. In some embodiments, Form A obtained according to Example 2 is about 95% to about 99.9% pure by weight.

In some embodiments, Form A is at least about 95% pure by weight, and Form A comprises no more than about 5% of an impurity by weight of Form A. In some embodiments, Form A is about 95.0% to 100% pure by weight, and Form A comprises 0% to about 5% of an impurity by weight of Form A. In some embodiments, Form A is about 98% to 100% pure by weight, and Form A comprises 0% to about 2% of an impurity by weight of Form A. In some embodiments, Form A is about 98%, about 98.5%, about 99%, about 99.5%, or 100% pure by weight, and Form A comprises about 2%, about 1.5%, about 1%, about 0.5%, about 0.25%, about 0.1%, about 0.05%, or 0%, respectively, of an impurity by weight of Form A. In some embodiments, Form A is about 99.5%, about 99.9%, or about 99.95% pure by weight, and Form A comprises about 0.5%, about 0.1%, or about 0.05%, respectively, of an impurity by weight of Form A.

In some embodiments, the purity or the impurity are determined by high-performance liquid chromatography (HPLC).

In some embodiments, Form A comprises less than 5% of each of other forms of Compound I selected from an amorphous form, Form B, Form C, Form D, Form E, Form F, Form G, Form H, Form I, Form J, Form K, or Form L. In some embodiments, Form A comprises less than 3% of each of other forms of Compound I selected from an amorphous form, Form B, Form C, Form D, Form E, Form F, Form G, Form H, Form I, Form J, Form K, or Form L. In some embodiments, Form A comprises less than 2% of each of other forms of Compound I selected from an amorphous form, Form B, Form C, Form D, Form E, Form F, Form G, Form H, Form I, Form J, Form K, or Form L. In some embodiments, Form A comprises less than 1% of each of other forms of Compound I selected from an amorphous form, Form B, Form C, Form D, Form E, Form F, Form G, Form H, Form I, Form J, Form K, or Form L. In some embodiments, Form A is purified. In some embodiments, Form A is isolated. In some embodiments, Form A is purified and isolated.

### Form B of Compound I

In one embodiment, the present disclosure relates to a Form B of Compound I or a pharmaceutically acceptable salt, solvate, or solvate salt thereof. In one embodiment, Form B is crystalline.

In some embodiments, Form B exhibits an X-ray powder diffraction (XRPD) pattern comprising a peak at about 2.8±0.2, about 8.4±0.2, and about 19.9±0.2 degrees two-theta. In some embodiments, Form B exhibits an XRPD pattern further comprising a peak at about 20.3±0.2, about 9.9±0.2, and/or about 13.9±0.2 degrees two-theta. In some embodiments, Form B exhibits an XRPD pattern further comprising a peak at about 19.1±0.2, about 9.1±0.2, and/or about 12.3±0.2 degrees two-theta.

In some embodiments, Form B exhibits an XRPD pattern comprising 1 peak, 2 peaks, 3 peaks, 4 peaks, 5 peaks, 6 peaks, 7 peaks, 8 peaks, 9 peaks, or 10 peaks of Table B. In some embodiments, Form B exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.8±0.2, about 8.4±0.2, 9.1±0.2, about 9.9±0.2, about 11.3±0.2, about 12.3±0.2, about 13.9±0.2, about 19.1±0.2, about 19.9±0.2, and about 20.3±0.2 degrees two-theta.

In some embodiments, Form B exhibits an XRPD pattern comprising a peak from Table B having an intensity (I %) of greater than 2%. In some embodiments, Form B exhibits an XRPD pattern comprising a peak from Table B having an intensity (I %) of greater than 1%. In some embodiments, Form B exhibits an XRPD pattern comprising a peak from Table B having an intensity (I %) of greater than 0.5%.

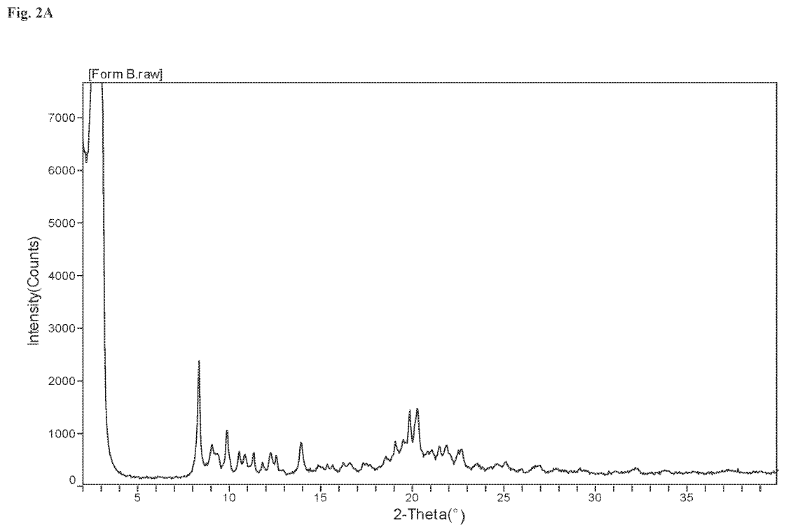

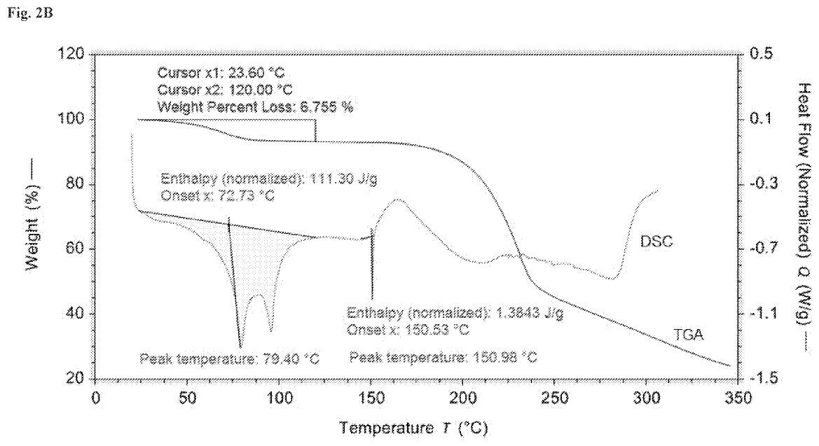

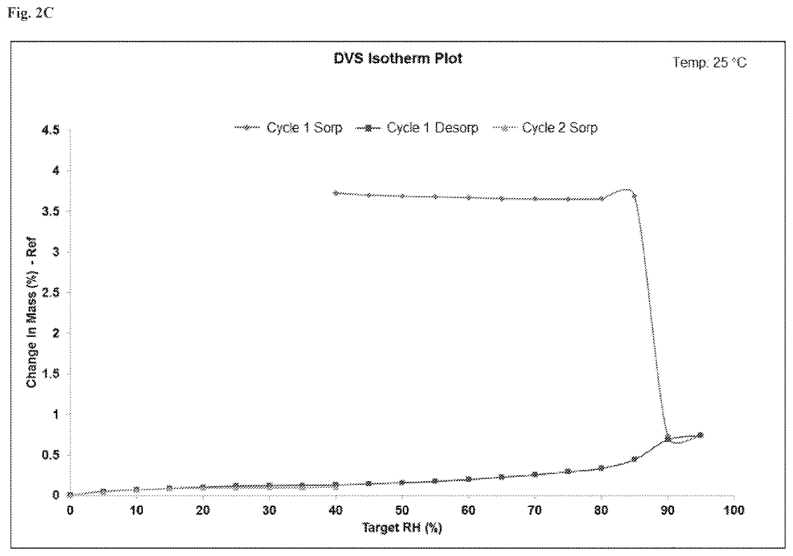

In some embodiments, Form B exhibits an XRPD pattern that is substantially the same as that depicted in FIG. 2A.

TABLE B
XRPD Table of Form B of Compound I

| # | 2-Theta | d(A) | BG | Height | I % |
|---|---|---|---|---|---|
| 1 | 2.833 | 31.1592 | 272 | 40581 | 100.0 |
| 2 | 8.347 | 10.5840 | 147 | 2225 | 5.5 |
| 3 | 9.059 | 9.7539 | 403 | 371 | 0.9 |
| 4 | 9.875 | 8.9493 | 367 | 670 | 1.7 |
| 5 | 10.555 | 8.3748 | 296 | 344 | 0.8 |
| 6 | 11.333 | 7.8014 | 275 | 340 | 0.8 |
| 7 | 12.265 | 7.2106 | 249 | 366 | 0.9 |
| 8 | 13.921 | 6.3561 | 262 | 552 | 1.4 |
| 9 | 14.877 | 5.9498 | 280 | 105 | 0.3 |
| 10 | 15.651 | 5.6572 | 277 | 101 | 0.2 |
| 11 | 16.568 | 5.3461 | 276 | 147 | 0.4 |
| 12 | 17.335 | 5.1114 | 285 | 137 | 0.3 |
| 13 | 19.076 | 4.6486 | 314 | 516 | 1.3 |
| 14 | 19.864 | 4.4660 | 562 | 865 | 2.1 |
| 15 | 20.266 | 4.3783 | 627 | 832 | 2.1 |
| 16 | 21.489 | 4.1318 | 559 | 181 | 0.4 |
| 17 | 21.866 | 4.0613 | 517 | 247 | 0.6 |
| 18 | 22.676 | 3.9181 | 416 | 268 | 0.7 |
| 19 | 23.566 | 3.7721 | 316 | 101 | 0.2 |
| 20 | 24.624 | 3.6124 | 292 | 106 | 0.3 |
| 21 | 25.118 | 3.5424 | 273 | 171 | 0.4 |
| 22 | 26.963 | 3.3041 | 245 | 134 | 0.3 |
| 23 | 27.904 | 3.1947 | 249 | 66 | 0.2 |
| 24 | 29.162 | 3.0597 | 236 | 86 | 0.2 |
| 25 | 32.185 | 2.7789 | 230 | 102 | 0.3 |
| 26 | 33.785 | 2.6509 | 226 | 53 | 0.1 |

In some embodiments, Form B has a melting range from about 153 °C to about 168 °C.

In some embodiments, Form B exhibits a differential scanning calorimetry (DSC) thermogram comprising an endothermic peak having a peak maximum of from about 70 °C to about 85 °C. In some embodiments, Form B exhibits a DSC thermogram comprising an endothermic peak having a peak maximum of from about 77 °C to about 82 °C. In some embodiments, Form B exhibits a DSC thermogram comprising an endothermic peak having a peak maximum of about 79 °C. In some embodiments, Form B exhibits a DSC thermogram comprising an endothermic peak that onsets at about 73 °C.

In some embodiments, Form B exhibits a differential scanning calorimetry (DSC) thermogram comprising an endothermic peak having a peak maximum of from about 148 °C to about 153 °C. In some embodiments, Form B exhibits a DSC thermogram comprising an endothermic peak having a peak maximum of from about 150 °C to about 152 °C. In some embodiments, Form B exhibits a DSC thermogram comprising an endothermic peak having a peak maximum of about 151 °C. In some embodiments, Form B exhibits a DSC thermogram comprising an endothermic peak that onsets at about 150 °C.

In some embodiments, Form B exhibits weight percent loss of about 6.8% by thermogravimetric analysis (TGA). In some embodiments, Form B exhibits weight percent loss of about 6.8% by TGA from room temperature to about 120 °C.

In some embodiments, Form B exhibits a DSC thermogram that is substantially the same as that depicted in FIG. 2B. In some embodiments, Form B exhibits a TGA thermogram that is substantially the same as that depicted in FIG. 2B.

In some embodiments, the water content of Form B is about 2.2% by volumetric Karl Fischer titration.

In some embodiments, Form B comprises about 0.3 molar equivalent of MTBE (4.76% by weight) by ¹H NMR.

In some embodiments, Form B water uptake from 40% RH to 80% RH at 25 °C is less than about 1% by weight. In some embodiments, Form B water uptake from 40% RH to 80% RH at 25 °C is less than about 0.5% by weight. In some embodiments, Form B water uptake from 40% RH to 80% RH at 25 °C is less than about 0.2% by weight. In some embodiments, Form B water uptake from 40% RH to 80% RH at 25 °C is less than about 0.1% by weight. In some embodiments, Form B is non-hygroscopic. In some embodiments, water uptake is measured by dynamic vapor sorption (DVS).

In some embodiments, Form B exhibits a DVS spectrum that is substantially the same as that depicted in FIG. 2C.

In some embodiments, Form B is purified. In some embodiments, Form B is isolated. In some embodiments, Form B is purified and isolated.

In some embodiments, Form B is at least about 90% pure by weight. In some embodiments, Form B is at least about 95% pure by weight. In some embodiments, Form B is at least about 95% pure by weight. In some embodiments, Form B is at least about 99% pure by weight. In some embodiments, Form B is at least about 90%, about 91%, about 92%, about 93%, about 94%, about 95%, about 96%, about 97%, about 98%, or about 99% pure by weight.

In some embodiments, Form B obtained according to Example 3 is at least about 90% pure by weight. In some embodiments, Form B obtained according to Example 3 is at least about 95% pure by weight. In some embodiments, Form B obtained according to Example 3 is at least about 95% pure by weight. In some embodiments, Form B obtained according to Example 3 is at least about 99% pure by weight.

In some embodiments, Form B is at least about 95% pure by weight, and Form B comprises no more than about 5% of an impurity by weight of Form B. In some embodiments. Form B is about 95.0% to 100% pure by weight, and Form B comprises 0% to about 5% of an impurity by weight of Form B. In some embodiments, Form B is about 98% to 100% pure by weight, and Form B comprises 0% to about 2% of an impurity by weight of Form B. In some embodiments, Form B is about 98%, about 98.5%, about 99%, about 99.5%, or 100% pure by weight, and Form B comprises about 2%, about 1.5%, about 1%, about 0.5%, or 0%, respectively, of an impurity by weight of Form B. In some embodiments, Form B is about 99.5%, about 99.9%, or about 99.95% pure by weight, and Form B comprises about 0.5%, about 0.1%, or about 0.05%, respectively, of an impurity by weight of Form B. In some embodiments, the purity or the impurity are determined by high-performance liquid chromatography (HPLC).

In some embodiments, Form B comprises less than 5% of each of other forms of Compound I selected from an amorphous form, Form A, Form C, Form D, Form E, Form F, Form G, Form H, Form I, Form J, Form K, or Form L. In some embodiments, Form B comprises less than 3% of each of other forms of Compound I selected from an amorphous form, Form A, Form C, Form D, Form E, Form F, Form G, Form H, Form I, Form J, Form K. or Form L. In some embodiments, Form B comprises less than 2% of each of other forms of Compound I selected from an amorphous form, Form A, Form C, Form D, Form E, Form F, Form G, Form H, Form I, Form J, Form K, or Form L. In some embodiments, Form B comprises less than 1% of each of other forms of Compound I selected from an amorphous form, Form A, Form C, Form D, Form E, Form F, Form G, Form H, Form I, Form J, Form K, or Form L. In some embodiments, Form B is purified. In some embodiments, Form B is isolated. In some embodiments, Form B is purified and isolated.

In some embodiments, Form B converts to Form A after DVS experiment.

In some embodiments, Form B comprises Form A in an amount ranging from about 1% to about 90% by weight, including all subranges and values therebetween. In some embodiments, Form B comprises Form A in an amount ranging from about 5% to about 80% by weight, including all subranges and values therebetween. In some embodiments, Form B comprises Form A in an amount ranging from about 5% to about 50% by weight, including all subranges and values therebetween.

### Form C of Compound I

In one embodiment, the present disclosure relates to a crystalline Form C of Compound I or a pharmaceutically acceptable salt, solvate, or solvate salt thereof.

In some embodiments, Form C exhibits an X-ray powder diffraction (XRPD) pattern comprising a peak at about 2.9±0.2, about 8.5±0.2, and about 17.0±0.2 degrees two-theta. In some embodiments, Form C exhibits an XRPD pattern further comprising a peak at about 19.9±0.2, about 14.1±0.2, and/or about 22.9±0.2 degrees two-theta. In some embodiments, Form C exhibits an XRPD pattern further comprising a peak at about 10.6±0.2, about 19.3±0.2, and/or about 25.6±0.2 degrees two-theta.

In some embodiments, Form C exhibits an XRPD pattern comprising 1 peak, 2 peaks, 3 peaks, 4 peaks, 5 peaks, 6 peaks, 7 peaks, 8 peaks, 9 peaks, or 10 peaks of Table C. In some embodiments, Form C exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.9±0.2, about 5.7±0.2, about 8.5±0.2, about 10.6±0.2, about 14.1±0.2, about 17.0±0.2, about 19.3±0.2, about 19.9±0.2, about 22.9±0.2, and about 25.6±0.2 degrees two-theta.

In some embodiments, Form C exhibits an XRPD pattern comprising a peak from Table C having an intensity (I %) of greater than 7%. In some embodiments, Form C exhibits an XRPD pattern comprising a peak from Table C having an intensity (I %) of greater than 0.5%. In some embodiments, Form C exhibits an XRPD pattern comprising a peak from Table C having an intensity (I %) of greater than 0.2%.

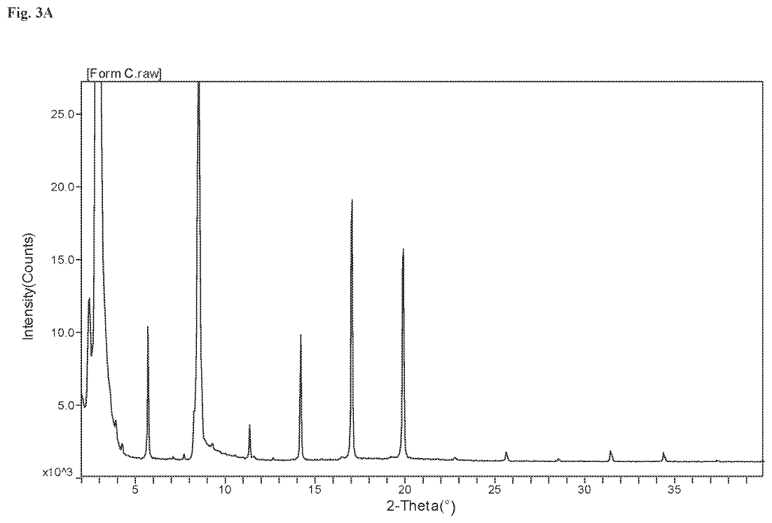

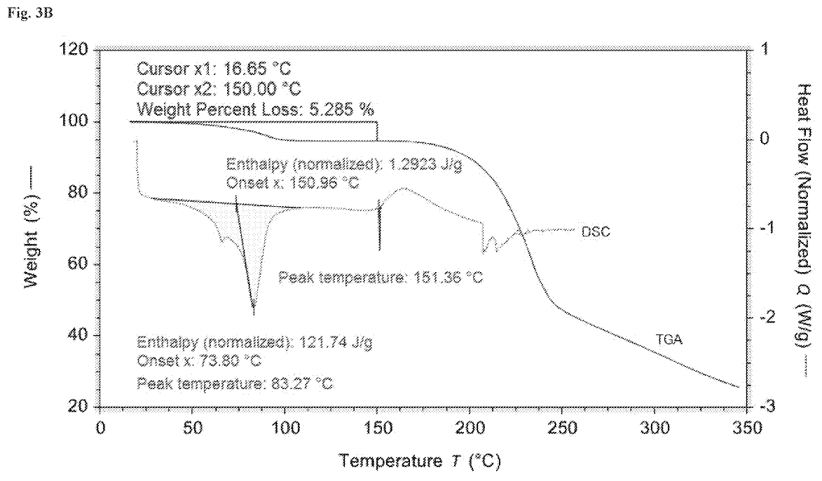

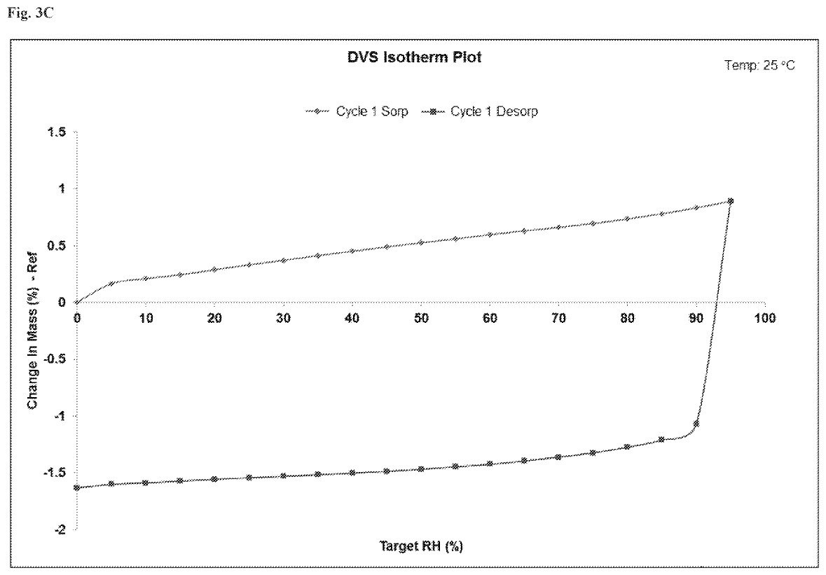

In some embodiments, Form C exhibits an XRPD pattern that is substantially the same as that depicted in FIG. 3A.

TABLE C
XRPD Table of Form C of Compound I

| # | 2-Theta | d(A) | BG | Height | I % |
|---|---|---|---|---|---|
| 1 | 2.875 | 30.7053 | 357 | 196398 | 100.0 |
| 2 | 5.694 | 15.5084 | 128 | 426 | 0.2 |
| 3 | 8.511 | 10.3801 | 136 | 14111 | 7.2 |
| 4 | 10.599 | 8.3399 | 210 | 595 | 0.3 |
| 5 | 10.916 | 8.0985 | 206 | 239 | 0.1 |
| 6 | 11.363 | 7.7806 | 204 | 166 | 0.1 |
| 7 | 12.317 | 7.1799 | 185 | 295 | 0.2 |
| 8 | 14.077 | 6.2859 | 187 | 752 | 0.4 |
| 9 | 16.457 | 5.3821 | 206 | 472 | 0.2 |
| 10 | 17.044 | 5.1980 | 197 | 1178 | 0.6 |
| 11 | 19.262 | 4.6042 | 342 | 594 | 0.3 |
| 12 | 19.896 | 4.4589 | 377 | 989 | 0.5 |
| 13 | 20.203 | 4.3917 | 374 | 440 | 0.2 |
| 14 | 21.865 | 4.0616 | 314 | 368 | 0.2 |
| 15 | 22.913 | 3.8780 | 249 | 726 | 0.4 |
| 16 | 24.810 | 3.5858 | 240 | 267 | 0.1 |
| 17 | 25.628 | 3.4730 | 234 | 615 | 0.3 |
| 18 | 27.172 | 3.2791 | 195 | 209 | 0.1 |
| 19 | 28.573 | 3.1214 | 191 | 203 | 0.1 |

In some embodiments, Form C has a melting range from about 153 °C to about 168 °C.

In some embodiments, Form C exhibits a differential scanning calorimetry (DSC) thermogram comprising an endothermic peak having a peak maximum of from about 70 °C to about 90 °C. In some embodiments, Form C exhibits a DSC thermogram comprising an endothermic peak having a peak maximum of from about 74 °C to about 86 °C. In some embodiments, Form C exhibits a DSC thermogram comprising an endothermic peak having a peak maximum of from about 82 °C to about 84 °C. In some embodiments, Form C exhibits a DSC thermogram comprising an endothermic peak having a peak maximum of about 83 °C. In some embodiments, Form C exhibits a DSC thermogram comprising an endothermic peak that onsets at about 74 °C.

In some embodiments, Form C exhibits a differential scanning calorimetry (DSC) thermogram comprising an endothermic peak having a peak maximum of from about 148 °C to about 153 °C. In some embodiments, Form C exhibits a DSC thermogram comprising an endothermic peak having a peak maximum of from about 150 °C to about 152 °C. In some embodiments, Form C exhibits a DSC thermogram comprising an endothermic peak having a peak maximum of about 151 °C. In some embodiments, Form C exhibits a DSC thermogram comprising an endothermic peak that onsets at about 151 °C.

In some embodiments, Form C exhibits weight percent loss of about 5.3% by thermogravimetric analysis (TGA). In some embodiments, Form C exhibits weight percent loss of about 5.3% by TGA from room temperature to about 150 °C.

In some embodiments, Form C exhibits a DSC thermogram that is substantially the same as that depicted in FIG. 3B. In some embodiments, Form C exhibits a TGA thermogram that is substantially the same as that depicted in FIG. 3B.

In some embodiments, the water content of Form C is about 2.2% by volumetric Karl Fischer titration.

In some embodiments, Form C comprises about 0.5 molar equivalent of ethanol (4.17% by weight) by ¹H NMR.

In some embodiments, Form C water uptake from 0% RH to 80% RH at 25 °C is about 0.2% by weight to about 3% by weight. In some embodiments, Form C water uptake from 0% RH to 80% RH at 25 °C is about 0.4% by weight to about 1.5% by weight. In some embodiments, Form C water uptake from 0% RH to 80% RH at 25 °C is about 0.4% by weight to about 1% by weight. In some embodiments, Form C water uptake from 0% RH to 80% RH at 25 °C is about 0.7% by weight. In some embodiments, Form C is slightly hygroscopic. In some embodiments, water uptake is measured by dynamic vapor sorption (DVS).

In some embodiments, Form C exhibits a DVS spectrum that is substantially the same as that depicted in FIG. 3C.

In some embodiments, Form C is purified. In some embodiments, Form C is isolated. In some embodiments, Form C is purified and isolated.

In some embodiments, Form C is at least about 90% pure by weight. In some embodiments, Form C is at least about 95% pure by weight. In some embodiments, Form C is at least about 95% pure by weight. In some embodiments, Form C is at least about 99% pure by weight. In some embodiments, Form C is at least about 90%, about 91%, about 92%, about 93%, about 94%, about 95%, about 96%, about 97%, about 98%, or about 99% pure by weight.

In some embodiments, Form C obtained according to Example 4 is at least about 90% pure by weight. In some embodiments, Form C obtained according to Example 4 is at least about 95% pure by weight. In some embodiments, Form C obtained according to Example 4 is at least about 95% pure by weight. In some embodiments, Form C obtained according to Example 4 is at least about 99% pure by weight.

In some embodiments, Form C is at least about 95% pure by weight, and Form C comprises no more than about 5% of an impurity by weight of Form C. In some embodiments. Form C is about 95.0% to 100% pure by weight, and Form C comprises 0% to about 5% of an impurity by weight of Form C. In some embodiments, Form C is about 98% to 100% pure by weight, and Form C comprises 0% to about 2% of an impurity by weight of Form C. In some embodiments, Form C is about 98%, about 98.5%, about 99%, about 99.5%, or 100% pure by weight, and Form C comprises about 2%, about 1.5%, about 1%, about 0.5%, or 0%, respectively, of an impurity by weight of Form C. In some embodiments, Form C is about 99.5%, about 99.9%, or about 99.95% pure by weight, and Form C comprises about 0.5%, about 0.1%, or about 0.05%, respectively, of an impurity by weight of Form C. In some embodiments, the purity or the impurity are determined by high-performance liquid chromatography (HPLC).

In some embodiments, Form C comprises less than 5% of each of other forms of Compound I selected from an amorphous form, Form A, Form B, Form D, Form E, Form F. Form G, Form H, Form 1, Form J, Form K, or Form L. In some embodiments, Form C comprises less than 3% of each of other forms of Compound I selected from an amorphous form, Form A, Form B, Form D, Form E, Form F, Form G, Form H, Form I, Form J, Form K, or Form L. In some embodiments, Form C comprises less than 2% of each of other forms of Compound I selected from an amorphous form, Form A, Form B, Form D, Form E, Form F, Form G, Form H, Form I, Form J, Form K, or Form L. In some embodiments, Form C comprises less than 1% of each of other forms of Compound I selected from an amorphous form, Form A, Form B, Form D, Form E, Form F, Form G, Form H, Form I, Form J, Form K, or Form L. In some embodiments, Form C is purified. In some embodiments, Form C is isolated. In some embodiments, Form C is purified and isolated.

In some embodiments, at least 50% of Form C converts to Form A after DVS experiment. In some embodiments, at least 70% of Form C converts to Form A after DVS experiment. In some embodiments, at least 80% of Form C converts to Form A after DVS experiment. In some embodiments, at least 90% of Form C converts to Form A after DVS experiment. In some embodiments, at least 95% of Form C converts to Form A after DVS experiment. In some embodiments, Form C converts to Form A after DVS experiment.

In some embodiments, Form C comprises Form A in an amount ranging from about 1% to about 90% by weight, including all subranges and values therebetween. In some embodiments, Form C comprises Form A in an amount ranging from about 5% to about 80% by weight, including all subranges and values therebetween. In some embodiments, Form C comprises Form A in an amount ranging from about 5% to about 50% by weight, including all subranges and values therebetween.

### Form D of Compound I

In one embodiment, the present disclosure relates to a Form D of Compound I or a pharmaceutically acceptable salt, solvate, or solvate salt thereof. In one embodiment, Form D is crystalline.

In some embodiments, Form D exhibits an X-ray powder diffraction (XRPD) pattern comprising a peak at about 2.8±0.2, about 3.9±0.2, and about 8.2±0.2 degrees two-theta. In some embodiments, Form D exhibits an XRPD pattern further comprising a peak at about 15.4±0.2, about 11.5±0.2, and/or about 7.7±0.2 degrees two-theta. In some embodiments, Form D exhibits an XRPD pattern further comprising a peak at about 19.2±0.2, about 16.4±0.2, and/or about 34.9±0.2 degrees two-theta.

In some embodiments, Form D exhibits an XRPD pattern comprising 1 peak, 2 peaks, 3 peaks, 4 peaks, 5 peaks, 6 peaks, 7 peaks, 8 peaks, 9 peaks, or 10 peaks of Table D. In some embodiments, Form D exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.8±0.2, about 3.9±0.2, about 7.1±0.2, about 8.2±0.2, about 9.9±0.2, about 11.5±0.2, about 15.4±0.2, about 16.4±0.2, about 19.2±0.2, and about 34.9±0.2 degrees two-theta.

In some embodiments, Form D exhibits an XRPD pattern comprising a peak from Table D having an intensity (I %) of greater than 50%. In some embodiments, Form D exhibits an XRPD pattern comprising a peak from Table D having an intensity (I %) of greater than 10%. In some embodiments, Form D exhibits an XRPD pattern comprising a peak from Table D having an intensity (I %) of greater than 5%. In some embodiments, Form D exhibits an XRPD pattern comprising a peak from Table D having an intensity (I %) of greater than 3%. In some embodiments, Form D exhibits an XRPD pattern comprising a peak from Table D having an intensity (I %) of greater than 0.5%.

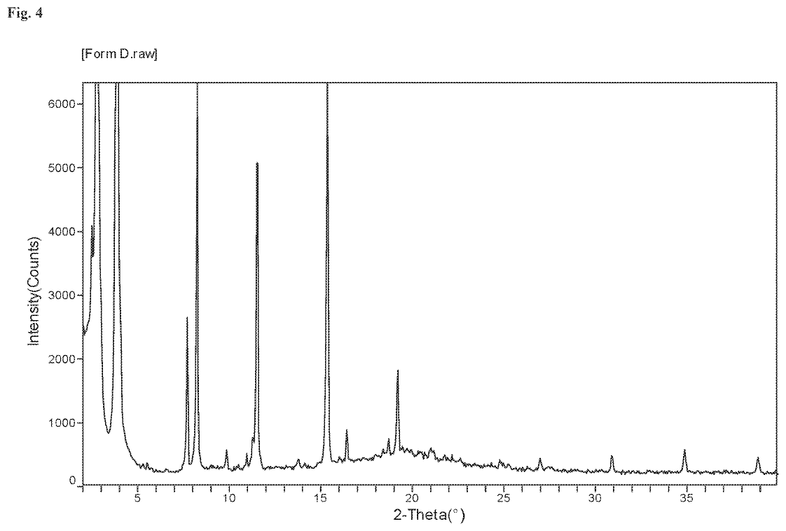

In some embodiments, Form D exhibits an XRPD pattern that is substantially the same as that depicted in FIG. 4.

TABLE D
XRPD Table of Form D of Compound I

| # | 2-Theta | d(A) | BG | Height | I % |
|---|---|---|---|---|---|
| 1 | 2.813 | 31.3824 | 906 | 49459 | 64.2 |
| 2 | 3.897 | 22.6574 | 349 | 77004 | 100.0 |
| 3 | 7.704 | 11.4665 | 240 | 2406 | 3.1 |
| 4 | 8.243 | 10.7175 | 252 | 8541 | 11.1 |
| 5 | 9.850 | 8.9718 | 253 | 301 | 0.4 |
| 6 | 10.954 | 8.0702 | 259 | 242 | 0.3 |
| 7 | 11.521 | 7.6744 | 271 | 4816 | 6.3 |
| 8 | 13.768 | 6.4267 | 291 | 116 | 0.1 |
| 9 | 15.357 | 5.7648 | 351 | 7999 | 10.4 |
| 10 | 16.427 | 5.3918 | 398 | 477 | 0.6 |
| 11 | 18.699 | 4.7414 | 496 | 235 | 0.3 |
| 12 | 19.202 | 4.6184 | 484 | 1332 | 1.7 |
| 13 | 21.020 | 4.2228 | 445 | 145 | 0.2 |
| 14 | 21.767 | 4.0797 | 388 | 90 | 0.1 |
| 15 | 24.805 | 3.5864 | 286 | 115 | 0.1 |
| 16 | 26.986 | 3.3013 | 239 | 183 | 0.2 |
| 17 | 30.903 | 2.8912 | 210 | 251 | 0.3 |
| 18 | 34.880 | 2.5701 | 207 | 357 | 0.5 |
| 19 | 38.889 | 2.3139 | 193 | 245 | 0.3 |

In some embodiments, Form D is purified. In some embodiments, Form D is isolated. In some embodiments, Form D is purified and isolated.

In some embodiments, Form D is at least about 90% pure by weight. In some embodiments, Form D is at least about 95% pure by weight. In some embodiments, Form D is at least about 95% pure by weight. In some embodiments, Form D is at least about 99% pure by weight. In some embodiments, Form D is at least about 90%, about 91%, about 92%, about 93%, about 94%, about 95%, about 96%, about 97%, about 98%, or about 99% pure by weight.

In some embodiments, Form D obtained according to Example 5 is at least about 90% pure by weight. In some embodiments, Form D obtained according to Example 5 is at least about 95% pure by weight. In some embodiments, Form D obtained according to Example 5 is at least about 95% pure by weight. In some embodiments, Form D obtained according to Example 5 is at least about 99% pure by weight.

In some embodiments, Form D is at least about 95% pure by weight, and Form D comprises no more than about 5% of an impurity by weight of Form D. In some embodiments, Form D is about 95.0% to 100% pure by weight, and Form D comprises 0% to about 5% of an impurity by weight of Form D. In some embodiments, Form D is about 98% to 100% pure by weight, and Form D comprises 0% to about 2% of an impurity by weight of Form C. In some embodiments, Form D is about 98%, about 98.5%, about 99%, about 99.5%, or 100% pure by weight, and Form D comprises about 2%, about 1.5%, about 1%, about 0.5%, or 0%, respectively, of an impurity by weight of Form D. In some embodiments, Form D is about 99.5%, about 99.9%, or about 99.95% pure by weight, and Form D comprises about 0.5%, about 0.1%, or about 0.05%, respectively, of an impurity by weight of Form D. In some embodiments, the purity or the impurity are determined by high-performance liquid chromatography (HPLC).

In some embodiments, Form D comprises less than 5% of each of other forms of Compound I selected from an amorphous form, Form A, Form B, Form C, Form E, Form F, Form G, Form H, Form I, Form J, Form K, or Form L. In some embodiments, Form D comprises less than 3% of each of other forms of Compound I selected from an amorphous form, Form A, Form B, Form C, Form E, Form F, Form G, Form H, Form I, Form J, Form K, or Form L. In some embodiments, Form D comprises less than 2% of each of other forms of Compound I selected from an amorphous form, Form A, Form B, Form C, Form E, Form F. Form G, Form H, Form I, Form J, Form K, or Form L. In some embodiments, Form D comprises less than 1% of each of other forms of Compound I selected from an amorphous form, Form A, Form B, Form C, Form E, Form F, Form G, Form H, Form I, Form J, Form K, or Form L. In some embodiments, Form D is purified. In some embodiments, Form D is isolated. In some embodiments, Form D is purified and isolated.

In some embodiments, Form D comprises Form J in an amount ranging from about 1% to about 90% by weight, including all subranges and values therebetween. In some embodiments, Form D comprises Form J in an amount ranging from about 5% to about 80% by weight, including all subranges and values therebetween. In some embodiments, Form D comprises Form J in an amount ranging from about 5% to about 50% by weight, including all subranges and values therebetween.

### Form E of Compound I

In one embodiment, the present disclosure relates to a Form E of Compound I or a pharmaceutically acceptable salt, solvate, or solvate salt thereof. In one embodiment, Form E is crystalline.

In some embodiments, Form E exhibits an X-ray powder diffraction (XRPD) pattern comprising a peak at about 3.1±0.2, about 9.3±0.2, and about 18.6±0.2 degrees two-theta. In some embodiments, Form E exhibits an XRPD pattern further comprising a peak at about 19.3±0.2, about 6.2±0.2, and/or about 21.8±0.2 degrees two-theta. In some embodiments, Form E exhibits an XRPD pattern further comprising a peak at about 24.8±0.2, about 12.4±0.2, and/or about 15.0±0.2 degrees two-theta.

In some embodiments, Form E exhibits an XRPD pattern comprising 1 peak, 2 peaks, 3 peaks, 4 peaks, 5 peaks, 6 peaks, 7 peaks, 8 peaks, 9 peaks, or 10 peaks of Table E. In some embodiments, Form E exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 3.1±0.2, about 6.2±0.2, about 9.3±0.2, about 12.4±0.2, about 15.0±0.2, about 18.6±0.2, about 19.3±0.2, about 21.8±0.2, about 24.8±0.2, and about 26.5±0.2 degrees two-theta.

In some embodiments, Form E exhibits an XRPD pattern comprising a peak from Table E having an intensity (I %) of greater than 8%. In some embodiments, Form E exhibits an XRPD pattern comprising a peak from Table E having an intensity (I %) of greater than 5%. In some embodiments, Form E exhibits an XRPD pattern comprising a peak from Table E having an intensity (I %) of greater than 1%. In some embodiments, Form E exhibits an XRPD pattern comprising a peak from Table E having an intensity (I %) of greater than 0.5%. In some embodiments, Form E exhibits an XRPD pattern comprising a peak from Table E having an intensity (I %) of greater than 0.2%.

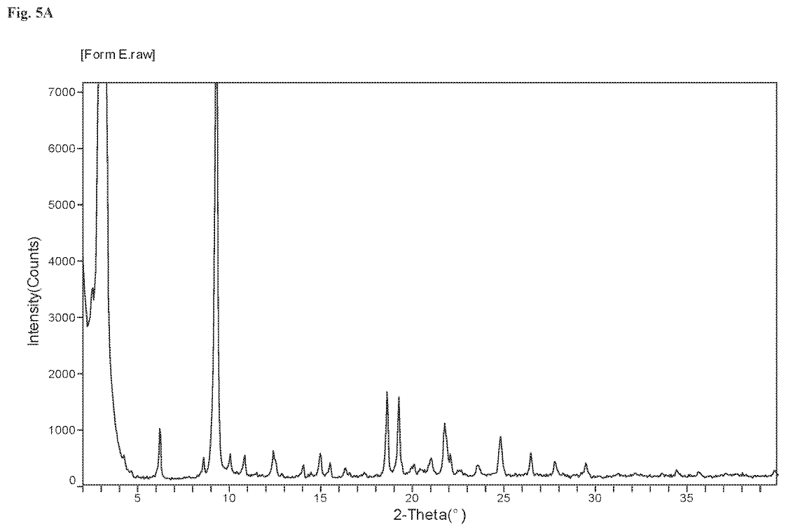

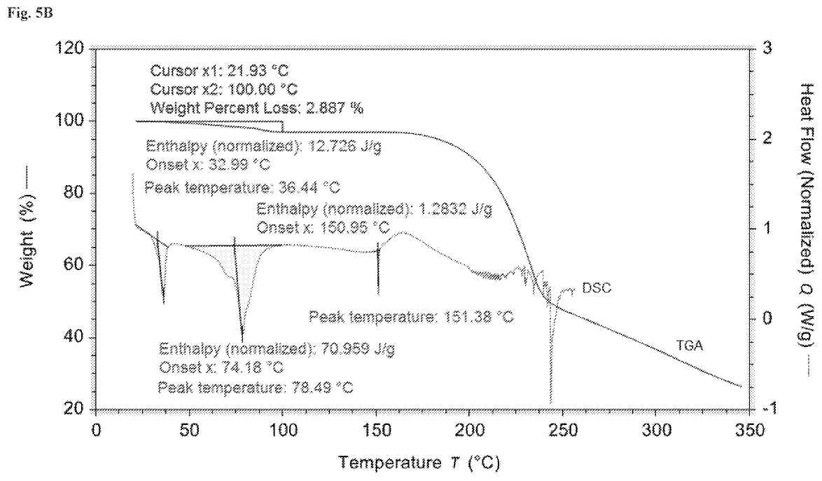

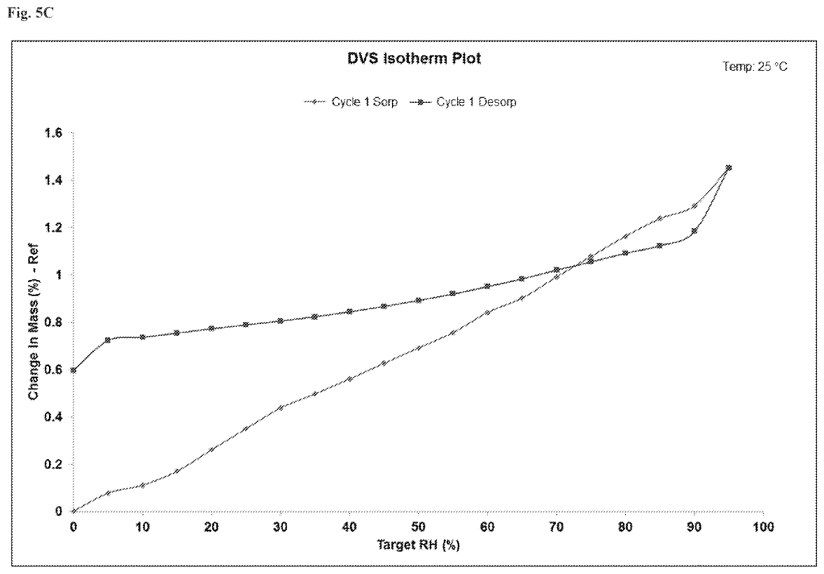

In some embodiments, Form E exhibits an XRPD pattern that is substantially the same as that depicted in FIG. 5A.

TABLE E
XRPD Table of Form E of Compound I

| # | 2-Theta | d(A) | BG | Height | I % |
|---|---|---|---|---|---|
| 1 | 3.138 | 28.1300 | 533 | 217704 | 100.0 |
| 2 | 4.243 | 20.8073 | 135 | 403 | 0.2 |
| 3 | 6.210 | 14.2213 | 126 | 889 | 0.4 |
| 4 | 8.594 | 10.2807 | 253 | 240 | 0.1 |
| 5 | 9.305 | 9.4965 | 198 | 20422 | 9.4 |
| 6 | 10.044 | 8.7996 | 313 | 249 | 0.1 |
| 7 | 10.833 | 8.1604 | 209 | 323 | 0.1 |
| 8 | 12.422 | 7.1195 | 160 | 454 | 0.2 |
| 9 | 14.032 | 6.3063 | 162 | 196 | 0.1 |
| 10 | 14.968 | 5.9140 | 182 | 386 | 0.2 |
| 11 | 15.505 | 5.7102 | 182 | 217 | 0.1 |
| 12 | 16.348 | 5.4176 | 148 | 160 | 0.1 |
| 13 | 18.618 | 4.7619 | 192 | 1477 | 0.7 |
| 14 | 19.261 | 4.6043 | 224 | 1351 | 0.6 |
| 15 | 20.087 | 4.4169 | 240 | 131 | 0.1 |
| 16 | 21.020 | 4.2229 | 234 | 250 | 0.1 |
| 17 | 21.780 | 4.0773 | 222 | 882 | 0.4 |
| 18 | 23.580 | 3.7699 | 182 | 170 | 0.1 |
| 19 | 24.820 | 3.5842 | 181 | 684 | 0.3 |
| 20 | 26.479 | 3.3633 | 179 | 397 | 0.2 |
| 21 | 27.794 | 3.2071 | 168 | 256 | 0.1 |
| 22 | 29.482 | 3.0272 | 151 | 245 | 0.1 |
| 23 | 34.452 | 2.6011 | 165 | 106 | 0.0 |
| 24 | 35.648 | 2.5165 | 158 | 79 | 0.0 |

In some embodiments, Form E has a melting range from about 152 °C to about 163 °C.

In some embodiments, Form E exhibits a differential scanning calorimetry (DSC) thermogram comprising an endothermic peak having a peak maximum of from about 30 °C to about 40 °C. In some embodiments, Form E exhibits a DSC thermogram comprising an endothermic peak having a peak maximum of from about 32 °C to about 40 °C. In some embodiments, Form E exhibits a DSC thermogram comprising an endothermic peak having a peak maximum of from about 34 °C to about 38 °C. In some embodiments, Form E exhibits a DSC thermogram comprising an endothermic peak having a peak maximum of about 36 °C. In some embodiments, Form E exhibits a DSC thermogram comprising an endothermic peak that onsets at about 33 °C.

In some embodiments, Form E exhibits a differential scanning calorimetry (DSC) thermogram comprising an endothermic peak having a peak maximum of from about 70 °C to about 85 °C. In some embodiments, Form E exhibits a DSC thermogram comprising an endothermic peak having a peak maximum of from about 74 °C to about 86 °C. In some embodiments, Form E exhibits a DSC thermogram comprising an endothermic peak having a peak maximum of from about 76 °C to about 80 °C. In some embodiments, Form E exhibits a DSC thermogram comprising an endothermic peak having a peak maximum of about 78 °C. In some embodiments, Form E exhibits a DSC thermogram comprising an endothermic peak that onsets at about 74 °C.

In some embodiments, Form E exhibits a differential scanning calorimetry (DSC) thermogram comprising an endothermic peak having a peak maximum of from about 148 °C to about 153 °C. In some embodiments, Form E exhibits a DSC thermogram comprising an endothermic peak having a peak maximum of from about 150 °C to about 152 °C. In some embodiments, Form E exhibits a DSC thermogram comprising an endothermic peak having a peak maximum of about 151 °C. In some embodiments, Form E exhibits a DSC thermogram comprising an endothermic peak that onsets at about 151 °C.

In some embodiments, Form E exhibits weight percent loss of about 2.9% by thermogravimetric analysis (TGA). In some embodiments, Form E exhibits weight percent loss of about 2.9% by TGA from room temperature to about 100 °C.

In some embodiments, Form E exhibits a DSC thermogram that is substantially the same as that depicted in FIG. 5B. In some embodiments, Form E exhibits a TGA thermogram that is substantially the same as that depicted in FIG. 5B.

In some embodiments, the water content of Form E is about 2.8% by volumetric Karl Fischer titration.

In some embodiments, Form E comprises about 0.2 molar equivalent of methanol (1.20% by weight) by ¹H NMR.

In some embodiments, Form E water uptake from 0% RH to 80% RH at 25 °C is about 0.2% by weight to about 4% by weight. In some embodiments, Form E water uptake from 0% RH to 80% RH at 25 °C is about 0.5% by weight to about 3% by weight. In some embodiments, Form E water uptake from 0% RH to 80% RH at 25 °C is about 0.7% by weight to about 1.5% by weight. In some embodiments, Form E water uptake from 0% RH to 80% RH at 25 °C is about 1.2% by weight. In some embodiments, Form E is slightly hygroscopic. In some embodiments, water uptake is measured by dynamic vapor sorption (DVS).

In some embodiments, Form E exhibits a DVS spectrum that is substantially the same as that depicted in FIG. 5C.

In some embodiments, Form E is purified. In some embodiments, Form E is isolated. In some embodiments, Form E is purified and isolated.

In some embodiments, Form E is at least about 90% pure by weight. In some embodiments, Form E is at least about 95% pure by weight. In some embodiments, Form E is at least about 95% pure by weight. In some embodiments, Form E is at least about 99% pure by weight. In some embodiments, Form E is at least about 90%, about 91%, about 92%, about 93%, about 94%, about 95%, about 96%, about 97%, about 98%, or about 99% pure by weight.

In some embodiments, Form E obtained according to Example 6 is at least about 90% pure by weight. In some embodiments, Form E obtained according to Example 6 is at least about 95% pure by weight. In some embodiments, Form E obtained according to Example 6 is at least about 95% pure by weight. In some embodiments, Form E obtained according to Example 6 is at least about 99% pure by weight.

In some embodiments, Form E is at least about 95% pure by weight, and Form E comprises no more than about 5% of an impurity by weight of Form E. In some embodiments, Form E is about 95.0% to 100% pure by weight, and Form E comprises 0% to about 5% of an impurity by weight of Form E. In some embodiments, Form E is about 98% to 100% pure by weight, and Form E comprises 0% to about 2% of an impurity by weight of Form E. In some embodiments, Form E is about 98%, about 98.5%, about 99%, about 99.5%, or 100% pure by weight, and Form E comprises about 2%, about 1.5%, about 1%, about 0.5%, or 0%, respectively, of an impurity by weight of Form E. In some embodiments, Form E is about 99.5%, about 99.9%, or about 99.95% pure by weight, and Form E comprises about 0.5%, about 0.1%, or about 0.05%, respectively, of an impurity by weight of Form E. In some embodiments, the purity or the impurity are determined by high-performance liquid chromatography (HPLC).

In some embodiments, Form E comprises less than 5% of each of other forms of Compound I selected from an amorphous form, Form A, Form B, Form C, Form D, Form F, Form G, Form H, Form I, Form J, Form K, or Form L. In some embodiments, Form E comprises less than 3% of each of other forms of Compound I selected from an amorphous form, Form A, Form B, Form C, Form D, Form F, Form G, Form H, Form I, Form J, Form K, or Form L. In some embodiments, Form E comprises less than 2% of each of other forms of Compound I selected from an amorphous form, Form A, Form B, Form C, Form D, Form F. Form G, Form H, Form I, Form J, Form K, or Form L. In some embodiments, Form E comprises less than 1% of each of other forms of Compound I selected from an amorphous form, Form A, Form B, Form C, Form D, Form F, Form G, Form H, Form I, Form J, Form K, or Form L. In some embodiments, Form E is purified. In some embodiments, Form E is isolated. In some embodiments, Form E is purified and isolated.

In some embodiments, at least 50% of Form E converts to Form A after DVS experiment. In some embodiments, at least 70% of Form E converts to Form A after DVS experiment. In some embodiments, at least 80% of Form E converts to Form A after DVS experiment. In some embodiments, at least 90% of Form E converts to Form A after DVS experiment. In some embodiments, at least 95% of Form E converts to Form A after DVS experiment. In some embodiments, Form E converts to Form A after DVS experiment.

In some embodiments, Form E comprises Form A in an amount ranging from about 1% to about 90% by weight, including all subranges and values therebetween. In some embodiments, Form E comprises Form A in an amount ranging from about 5% to about 80% by weight, including all subranges and values therebetween. In some embodiments, Form E comprises Form A in an amount ranging from about 5% to about 50% by weight, including all subranges and values therebetween.

### Form F of Compound I

In one embodiment, the present disclosure relates to a Form F of Compound I or a pharmaceutically acceptable salt, solvate, or solvate salt thereof. In one embodiment, Form F is crystalline.

In some embodiments, Form F exhibits an X-ray powder diffraction (XRPD) pattern comprising a peak at about 2.9±0.2, about 8.5±0.2, and about 20.3±0.2 degrees two-theta. In some embodiments, Form F exhibits an XRPD pattern further comprising a peak at about 14.1±0.2, about 19.7±0.2, and/or about 10.0±0.2 degrees two-theta. In some embodiments, Form F exhibits an XRPD pattern further comprising a peak at about 10.6±0.2, about 22.7±0.2, and/or about 16.9±0.2 degrees two-theta.

In some embodiments, Form F exhibits an XRPD pattern comprising 1 peak, 2 peaks, 3 peaks, 4 peaks, 5 peaks, 6 peaks, 7 peaks, 8 peaks, 9 peaks, or 10 peaks of Table F. In some embodiments, Form F exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.9±0.2, about 8.5±0.2, about 9.3±0.2, about 10.0±0.2, about 10.6±0.2, about 14.1±0.2, about 16.9±0.2, about 19.7±0.2, about 20.3±0.2, and about 22.7±0.2 degrees two-theta.

In some embodiments, Form F exhibits an XRPD pattern comprising a peak from Table F having an intensity (I %) of greater than 5%. In some embodiments, Form F exhibits an XRPD pattern comprising a peak from Table F having an intensity (I %) of greater than 1%. In some embodiments, Form F exhibits an XRPD pattern comprising a peak from Table F having an intensity (I %) of greater than 0.5%. In some embodiments, Form F exhibits an XRPD pattern comprising a peak from Table F having an intensity (I %) of greater than 0.2%.

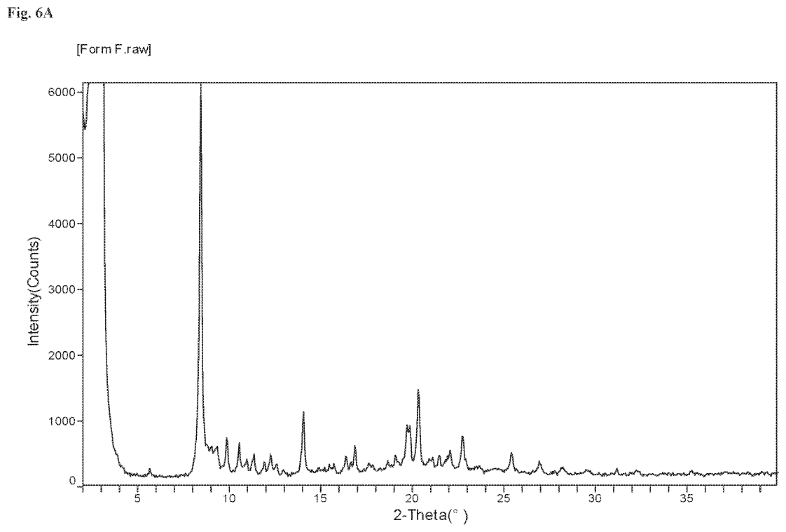

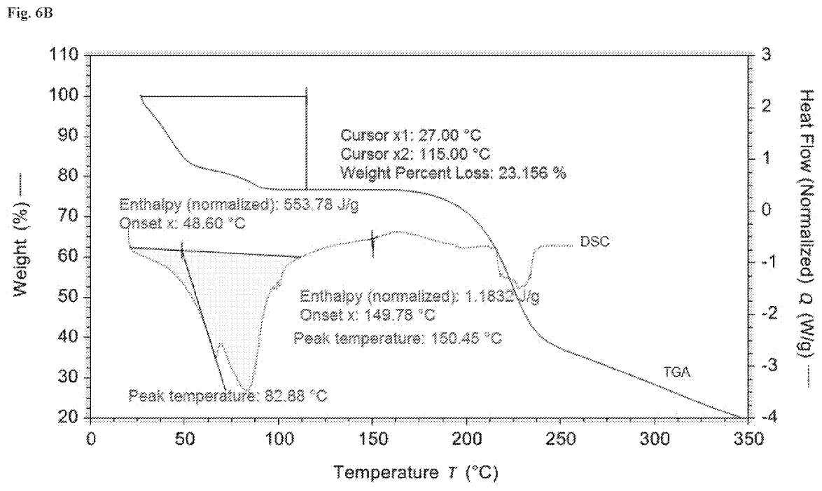

In some embodiments, Form F exhibits an XRPD pattern that is substantially the same as that depicted in FIG. 6A.

TABLE F
XRPD Table of Form F of Compound I

| # | 2-Theta | d(A) | BG | Height | I % |
|---|---|---|---|---|---|
| 1 | 2.852 | 30.9477 | 469 | 119637 | 100.0 |
| 2 | 8.448 | 10.4572 | 160 | 8396 | 7.0 |
| 3 | 9.339 | 9.4619 | 228 | 367 | 0.3 |
| 4 | 9.870 | 8.9543 | 290 | 438 | 0.4 |
| 5 | 10.549 | 8.3795 | 220 | 432 | 0.4 |
| 6 | 10.945 | 8.0773 | 212 | 189 | 0.2 |
| 7 | 11.337 | 7.7987 | 211 | 269 | 0.2 |
| 8 | 12.261 | 7.2130 | 187 | 291 | 0.2 |
| 9 | 12.580 | 7.0306 | 181 | 146 | 0.1 |
| 10 | 14.051 | 6.2976 | 196 | 928 | 0.8 |
| 11 | 15.704 | 5.6383 | 213 | 121 | 0.1 |
| 12 | 16.375 | 5.4089 | 219 | 232 | 0.2 |
| 13 | 16.866 | 5.2523 | 230 | 378 | 0.3 |
| 14 | 19.732 | 4.4955 | 324 | 603 | 0.5 |
| 15 | 20.330 | 4.3646 | 345 | 1119 | 0.9 |
| 16 | 22.055 | 4.0269 | 315 | 222 | 0.2 |
| 17 | 22.741 | 3.9071 | 275 | 476 | 0.4 |
| 18 | 25.421 | 3.5009 | 221 | 278 | 0.2 |
| 19 | 26.939 | 3.3070 | 196 | 180 | 0.2 |
| 20 | 29.491 | 3.0264 | 170 | 72 | 0.1 |

In some embodiments, Form F has a melting range from about 150 °C to about 163 °C.

In some embodiments, Form F exhibits a differential scanning calorimetry (DSC) thermogram comprising an endothermic peak having a peak maximum of from about 75 °C to about 90 °C. In some embodiments, Form F exhibits a DSC thermogram comprising an endothermic peak having a peak maximum of from about 78 °C to about 86 °C. In some embodiments, Form F exhibits a DSC thermogram comprising an endothermic peak having a peak maximum of from about 80 °C to about 85 °C. In some embodiments, Form F exhibits a DSC thermogram comprising an endothermic peak having a peak maximum of about 83 °C. In some embodiments, Form F exhibits a DSC thermogram comprising an endothermic peak that onsets at about 49 °C.

In some embodiments, Form F exhibits a differential scanning calorimetry (DSC) thermogram comprising an endothermic peak having a peak maximum of from about 148 °C to about 153 °C. In some embodiments, Form F exhibits a DSC thermogram comprising an endothermic peak having a peak maximum of from about 149 °C to about 152 °C. In some embodiments, Form F exhibits a DSC thermogram comprising an endothermic peak having a peak maximum of about 150 °C. In some embodiments, Form F exhibits a DSC thermogram comprising an endothermic peak that onsets at about 150 °C.

In some embodiments, Form F exhibits weight percent loss of about 23.2% by thermogravimetric analysis (TGA). In some embodiments, Form F exhibits weight percent loss of about 23.2% by TGA from room temperature to about 115 °C.

In some embodiments, Form F exhibits a DSC thermogram that is substantially the same as that depicted in FIG. 6B. In some embodiments, Form F exhibits a TGA thermogram that is substantially the same as that depicted in FIG. 6B.

In some embodiments, Form F comprises about 0.7 molar equivalent of THF (8.70% by weight) by ¹H NMR.

In some embodiments, Form F is purified. In some embodiments, Form F is isolated. In some embodiments, Form F is purified and isolated.

In some embodiments, Form F is at least about 90% pure by weight. In some embodiments, Form F is at least about 95% pure by weight. In some embodiments, Form F is at least about 95% pure by weight. In some embodiments, Form F is at least about 99% pure by weight. In some embodiments, Form F is at least about 90%, about 91%, about 92%, about 93%, about 94%, about 95%, about 96%, about 97%, about 98%, or about 99% pure by weight.

In some embodiments, Form F obtained according to Example 7 is at least about 90% pure by weight. In some embodiments, Form F obtained according to Example 7 is at least about 95% pure by weight. In some embodiments, Form F obtained according to Example 7 is at least about 95% pure by weight. In some embodiments, Form F obtained according to Example 7 is at least about 99% pure by weight.

In some embodiments, Form F is at least about 95% pure by weight, and Form F comprises no more than about 5% of an impurity by weight of Form F. In some embodiments, Form F is about 95.0% to 100% pure by weight, and Form F comprises 0% to about 5% of an impurity by weight of Form F. In some embodiments, Form F is about 98% to 100% pure by weight, and Form F comprises 0% to about 2% of an impurity by weight of Form F. In some embodiments, Form F is about 98%, about 98.5%, about 99%, about 99.5%, or 100% pure by weight, and Form F comprises about 2%, about 1.5%, about 1%, about 0.5%, or 0%, respectively, of an impurity by weight of Form F. In some embodiments, Form F is about 99.5%, about 99.9%, or about 99.95% pure by weight, and Form F comprises about 0.5%, about 0.1%, or about 0.05%, respectively, of an impurity by weight of Form F. In some embodiments, the purity or the impurity are determined by high-performance liquid chromatography (HPLC).

In some embodiments, Form F comprises less than 5% of each of other forms of Compound I selected from an amorphous form, Form A, Form B, Form C, Form D, Form E, Form G, Form H, Form I, Form J, Form K, or Form L. In some embodiments, Form F comprises less than 3% of each of other forms of Compound I selected from an amorphous form, Form A, Form B, Form C, Form D, Form E, Form G, Form H, Form I, Form J, Form K, or Form L. In some embodiments, Form F comprises less than 2% of each of other forms of Compound I selected from an amorphous form, Form A, Form B, Form C, Form D, Form E, Form G, Form H, Form I, Form J, Form K, or Form L. In some embodiments, Form F comprises less than 1% of each of other forms of Compound I selected from an amorphous form, Form A, Form B, Form C, Form D, Form E, Form G, Form H, Form I, Form J, Form K, or Form L. In some embodiments, Form F is purified. In some embodiments, Form F is isolated. In some embodiments, Form F is purified and isolated.

### Form G of Compound I

In one embodiment, the present disclosure relates to a Form G of Compound I or a pharmaceutically acceptable salt, solvate, or solvate salt thereof. In one embodiment, Form G is crystalline.

In some embodiments, Form G exhibits an X-ray powder diffraction (XRPD) pattern comprising a peak at about 2.8±0.2, about 8.3±0.2, and about 19.9±0.2 degrees two-theta. In some embodiments, Form G exhibits an XRPD pattern further comprising a peak at about 13.9±0.2, about 9.8±0.2, and/or about 10.5±0.2 degrees two-theta. In some embodiments, Form G exhibits an XRPD pattern further comprising a peak at about 11.3±0.2, about 19.3±0.2, and/or about 24.9±0.2 degrees two-theta.

In some embodiments, Form G exhibits an XRPD pattern comprising 1 peak, 2 peaks, 3 peaks, 4 peaks, 5 peaks, 6 peaks, 7 peaks, 8 peaks, 9 peaks, or 10 peaks of Table G. In some embodiments, Form G exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.8±0.2, about 8.3±0.2, about 9.8±0.2, about 10.5±0.2, about 11.3±0.2, about 13.9±0.2, about 19.3±0.2, about 19.9±0.2, about 20.3±0.2, and about 24.9±0.2 degrees two-theta.

In some embodiments, Form G exhibits an XRPD pattern comprising a peak from Table G having an intensity (I %) of greater than 5%. In some embodiments, Form G exhibits an XRPD pattern comprising a peak from Table G having an intensity (I %) of greater than 1%. In some embodiments, Form G exhibits an XRPD pattern comprising a peak from Table G having an intensity (I %) of greater than 0.5%. In some embodiments, Form G exhibits an XRPD pattern comprising a peak from Table G having an intensity (I %) of greater than 0.2%.

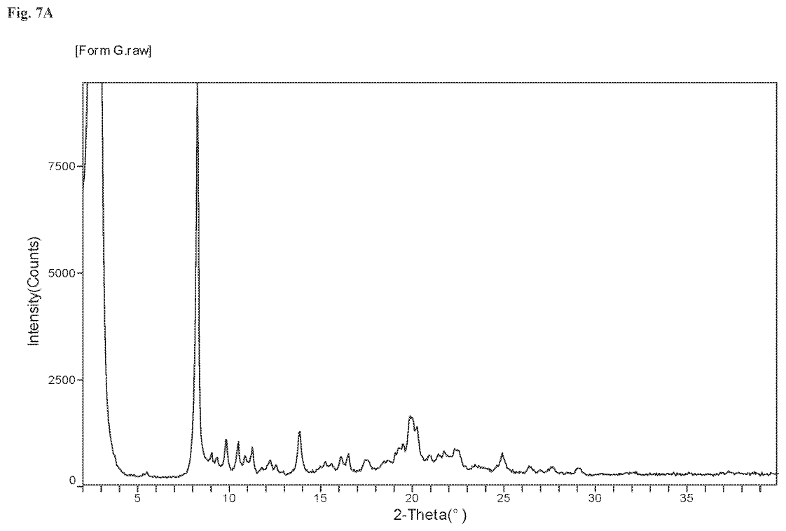

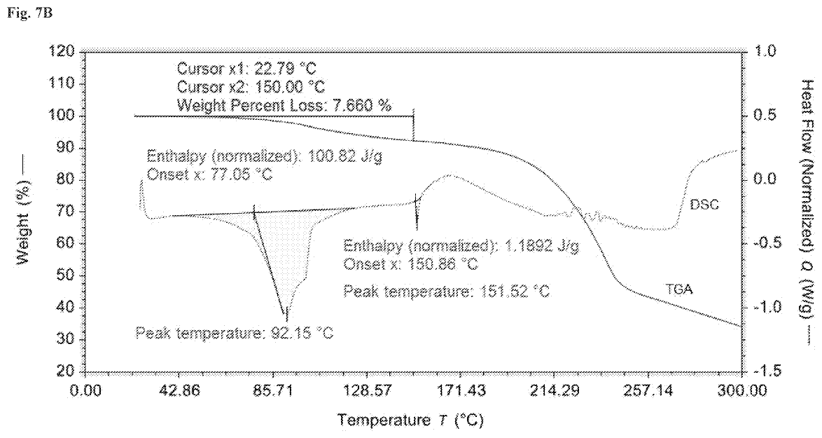

In some embodiments, Form G exhibits an XRPD pattern that is substantially the same as that depicted in FIG. 7A.

TABLE G
XRPD Table of Form G of Compound I

| # | 2-Theta | d(A) | BG | Height | 1% |
|---|---|---|---|---|---|
| 1 | 2.790 | 31.6394 | 538 | 166102 | 100.0 |
| 2 | 5.499 | 16.0589 | 203 | 108 | 0.1 |
| 3 | 8.255 | 10.7017 | 209 | 10121 | 6.1 |
| 4 | 9.035 | 9.7799 | 594 | 174 | 0.1 |
| 5 | 9.337 | 9.4642 | 576 | 81 | 0.0 |
| 6 | 9.824 | 8.9958 | 460 | 611 | 0.4 |
| 7 | 10.492 | 8.4248 | 396 | 627 | 0.4 |
| 8 | 10.868 | 8.1343 | 382 | 312 | 0.2 |
| 9 | 11.251 | 7.8577 | 370 | 535 | 0.3 |
| 10 | 12.250 | 7.2191 | 317 | 272 | 0.2 |
| 11 | 12.549 | 7.0481 | 301 | 181 | 0.1 |
| 12 | 13.852 | 6.3876 | 298 | 970 | 0.6 |
| 13 | 15.255 | 5.8032 | 367 | 190 | 0.1 |
| 14 | 15.558 | 5.6910 | 387 | 119 | 0.1 |
| 15 | 16.116 | 5.4952 | 385 | 286 | 0.2 |
| 16 | 16.501 | 5.3679 | 361 | 382 | 0.2 |
| 17 | 17.469 | 5.0726 | 366 | 232 | 0.1 |
| 18 | 19.274 | 4.6013 | 439 | 441 | 0.3 |
| 19 | 19.877 | 4.4630 | 607 | 1029 | 0.6 |
| 20 | 20.253 | 4.3809 | 809 | 572 | 0.3 |
| 21 | 20.947 | 4.2375 | 610 | 97 | 0.1 |
| 22 | 21.415 | 4.1458 | 666 | 69 | 0.0 |
| 23 | 21.736 | 4.0853 | 573 | 236 | 0.1 |
| 24 | 22.330 | 3.9779 | 488 | 373 | 0.2 |
| 25 | 23.426 | 3.7942 | 383 | 105 | 0.1 |
| 26 | 24.924 | 3.5696 | 334 | 427 | 0.3 |
| 27 | 26.389 | 3.3746 | 314 | 139 | 0.1 |
| 28 | 27.669 | 3.2214 | 300 | 134 | 0.1 |
| 29 | 29.124 | 3.0636 | 260 | 146 | 0.1 |

In some embodiments, Form G has a melting range from about 157 °C to about 166 °C.

In some embodiments, Form G exhibits a differential scanning calorimetry (DSC) thermogram comprising an endothermic peak having a peak maximum of from about 85 °C to about 100 °C. In some embodiments, Form G exhibits a DSC thermogram comprising an endothermic peak having a peak maximum of from about 88 °C to about 95 °C. In some embodiments, Form G exhibits a DSC thermogram comprising an endothermic peak having a peak maximum of from about 90 °C to about 94 °C. In some embodiments, Form G exhibits a DSC thermogram comprising an endothermic peak having a peak maximum of about 92 °C. In some embodiments, Form G exhibits a DSC thermogram comprising an endothermic peak that onsets at about 77 °C.

In some embodiments, Form G exhibits a differential scanning calorimetry (DSC) thermogram comprising an endothermic peak having a peak maximum of from about 148 °C to about 155 °C. In some embodiments, Form G exhibits a DSC thermogram comprising an endothermic peak having a peak maximum of from about 150 °C to about 153 °C. In some embodiments, Form G exhibits a DSC thermogram comprising an endothermic peak having a peak maximum of about 152 °C. In some embodiments, Form G exhibits a DSC thermogram comprising an endothermic peak that onsets at about 151 °C.

In some embodiments, Form G exhibits weight percent loss of about 7.7% by thermogravimetric analysis (TGA). In some embodiments, Form G exhibits weight percent loss of about 7.7% by TGA from room temperature to about 150 °C.

In some embodiments, Form G exhibits a DSC thermogram that is substantially the same as that depicted in FIG. 7B. In some embodiments, Form G exhibits a TGA thermogram that is substantially the same as that depicted in FIG. 7B.

In some embodiments, Form G comprises about 0.13 molar equivalent of 2-methyltetrahydrofuran (2.07% by weight) by ¹H NMR.

In some embodiments, Form G is purified. In some embodiments, Form G is isolated. In some embodiments, Form G is purified and isolated.

In some embodiments, Form G is at least about 90% pure by weight. In some embodiments, Form G is at least about 95% pure by weight. In some embodiments, Form G is at least about 95% pure by weight. In some embodiments, Form G is at least about 99% pure by weight. In some embodiments, Form G is at least about 90%, about 91%, about 92%, about 93%, about 94%, about 95%, about 96%, about 97%, about 98%, or about 99% pure by weight.

In some embodiments, Form G obtained according to Example 8 is at least about 90% pure by weight. In some embodiments, Form G obtained according to Example 8 is at least about 95% pure by weight. In some embodiments, Form G obtained according to Example 8 is at least about 95% pure by weight. In some embodiments, Form G obtained according to Example 8 is at least about 99% pure by weight.

In some embodiments, Form G is at least about 95% pure by weight, and Form G comprises no more than about 5% of an impurity by weight of Form G. In some embodiments, Form G is about 95.0% to 100% pure by weight, and Form G comprises 0% to about 5% of an impurity by weight of Form G. In some embodiments, Form G is about 98% to 100% pure by weight, and Form G comprises 0% to about 2% of an impurity by weight of Form G. In some embodiments, Form G is about 98%, about 98.5%, about 99%, about 99.5%, or 100% pure by weight, and Form G comprises about 2%, about 1.5%, about 1%, about 0.5%, or 0%, respectively, of an impurity by weight of Form G. In some embodiments, Form G is about 99.5%, about 99.9%, or about 99.95% pure by weight, and Form G comprises about 0.5%, about 0.1%, or about 0.05%, respectively, of an impurity by weight of Form G. In some embodiments, the purity or the impurity are determined by high-performance liquid chromatography (HPLC).

In some embodiments, Form G comprises less than 5% of each of other forms of Compound I selected from an amorphous form, Form A, Form B, Form C, Form D, Form E, Form F, Form H, Form I, Form J, Form K, or Form L. In some embodiments, Form G comprises less than 3% of each of other forms of Compound I selected from an amorphous form, Form A, Form B, Form C, Form D, Form E, Form F, Form H, Form I, Form J, Form K, or Form L. In some embodiments, Form G comprises less than 2% of each of other forms of Compound I selected from an amorphous form, Form A, Form B, Form C, Form D, Form E, Form F, Form H, Form I, Form J, Form K, or Form L. In some embodiments, Form G comprises less than 1% of each of other forms of Compound I selected from an amorphous form, Form A, Form B, Form C, Form D, Form E, Form F, Form H, Form I, Form J, Form K, or Form L. In some embodiments, Form G is purified. In some embodiments, Form G is isolated. In some embodiments, Form G is purified and isolated.

### Form H of Compound I

In one embodiment, the present disclosure relates to a Form H of Compound I or a pharmaceutically acceptable salt, solvate, or solvate salt thereof. In one embodiment, Form H is crystalline.

In some embodiments, Form H exhibits an X-ray powder diffraction (XRPD) pattern comprising a peak at about 2.8±0.2, about 8.1±0.2, and about 21.7±0.2 degrees two-theta. In some embodiments, Form H exhibits an XRPD pattern further comprising a peak at about 10.8±0.2, about 16.3±0.2, and/or about 19.0±0.2 degrees two-theta. In some embodiments, Form H exhibits an XRPD pattern further comprising a peak at about 5.4±0.2, about 19.8±0.2, and/or about 22.0±0.2 degrees two-theta.

In some embodiments, Form H exhibits an XRPD pattern comprising 1 peak, 2 peaks, 3 peaks, 4 peaks, 5 peaks, 6 peaks, 7 peaks, 8 peaks, 9 peaks, or 10 peaks of Table H. In some embodiments, Form H exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.8±0.2, about 5.4±0.2, about 8.1±0.2, about 10.8±0.2, about 16.3±0.2, about 19.0±0.2, about 19.8±0.2, about 21.7±0.2, about 22.0±0.2, and about 24.6±0.2 degrees two-theta.

In some embodiments, Form H exhibits an XRPD pattern comprising a peak from Table H having an intensity (I %) of greater than 5%. In some embodiments, Form H exhibits an XRPD pattern comprising a peak from Table H having an intensity (I %) of greater than 0.5%. In some embodiments, Form H exhibits an XRPD pattern comprising a peak from Table H having an intensity (I %) of greater than 0.2%.

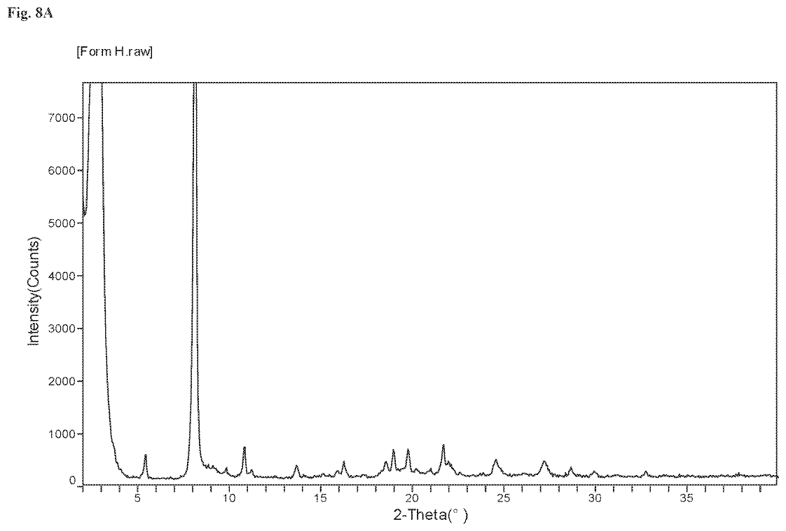

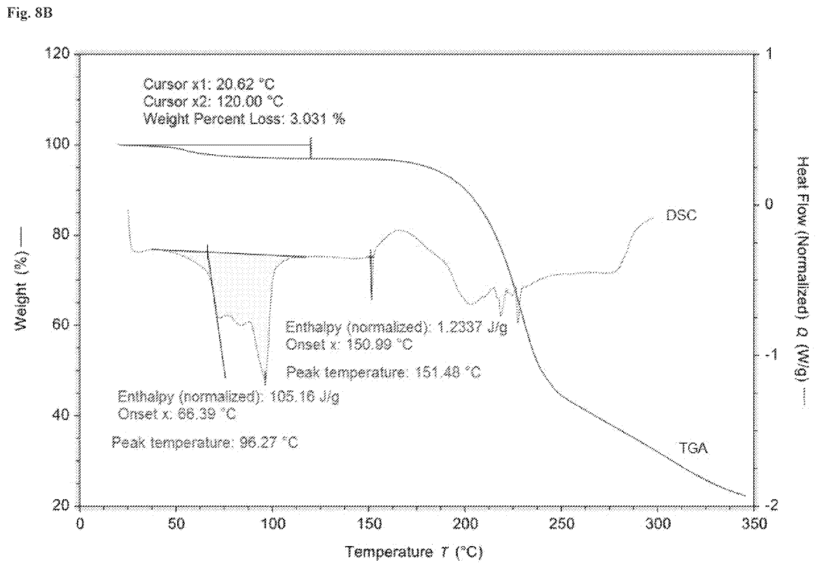

In some embodiments, Form H exhibits an XRPD pattern that is substantially the same as that depicted in FIG. 8A.

TABLE H
XRPD Table of Form H of Compound I

| # | 2-Theta | d(A) | BG | Height | 1% |
|---|---|---|---|---|---|
| 1 | 2.754 | 32.0507 | 555 | 365454 | 100.0 |
| 2 | 5.429 | 16.2660 | 152 | 429 | 0.1 |
| 3 | 8.142 | 10.8501 | 125 | 22576 | 6.2 |
| 4 | 10.835 | 8.1586 | 181 | 548 | 0.1 |
| 5 | 16.267 | 5.4443 | 165 | 295 | 0.1 |
| 6 | 18.972 | 4.6738 | 166 | 516 | 0.1 |
| 7 | 19.773 | 4.4862 | 280 | 410 | 0.1 |
| 8 | 21.707 | 4.0908 | 219 | 554 | 0.2 |
| 9 | 22.016 | 4.0339 | 206 | 211 | 0.1 |
| 10 | 24.571 | 3.6201 | 190 | 301 | 0.1 |
| 11 | 27.184 | 3.2777 | 189 | 274 | 0.1 |

In some embodiments, Form H has a melting range from about 154 °C to about 167 °C.

In some embodiments, Form H exhibits a differential scanning calorimetry (DSC) thermogram comprising an endothermic peak having a peak maximum of from about 90 °C to about 100 °C. In some embodiments, Form H exhibits a DSC thermogram comprising an endothermic peak having a peak maximum of from about 92 °C to about 100 °C. In some embodiments, Form H exhibits a DSC thermogram comprising an endothermic peak having a peak maximum of from about 94 °C to about 98 °C. In some embodiments, Form H exhibits a DSC thermogram comprising an endothermic peak having a peak maximum of about 96 °C. In some embodiments, Form H exhibits a DSC thermogram comprising an endothermic peak that onsets at about 66 °C.

In some embodiments, Form H exhibits a differential scanning calorimetry (DSC) thermogram comprising an endothermic peak having a peak maximum of from about 148 °C to about 155 °C. In some embodiments, Form H exhibits a DSC thermogram comprising an endothermic peak having a peak maximum of from about 149 °C to about 153 °C. In some embodiments, Form H exhibits a DSC thermogram comprising an endothermic peak having a peak maximum of about 151 °C. In some embodiments, Form H exhibits a DSC thermogram comprising an endothermic peak that onsets at about 151 °C.

In some embodiments, Form H exhibits weight percent loss of about 3.0% by thermogravimetric analysis (TGA). In some embodiments, Form H exhibits weight percent loss of about 3.0% by TGA from room temperature to about 120 °C.

In some embodiments, Form H exhibits a DSC thermogram that is substantially the same as that depicted in FIG. 8B. In some embodiments, Form H exhibits a TGA thermogram that is substantially the same as that depicted in FIG. 8B.

In some embodiments, Form H comprises about 0.3 molar equivalent of MTBE (4.76% by weight) by ¹H NMR.

In some embodiments, Form H is purified. In some embodiments, Form H is isolated. In some embodiments, Form H is purified and isolated.

In some embodiments, Form H is at least about 90% pure by weight. In some embodiments, Form H is at least about 95% pure by weight. In some embodiments, Form H is at least about 95% pure by weight. In some embodiments, Form H is at least about 99% pure by weight. In some embodiments, Form H is at least about 90%, about 91%, about 92%, about 93%, about 94%, about 95%, about 96%, about 97%, about 98%, or about 99% pure by weight.

In some embodiments, Form H obtained according to Example 9 is at least about 90% pure by weight. In some embodiments, Form H obtained according to Example 9 is at least about 95% pure by weight. In some embodiments, Form H obtained according to Example 9 is at least about 95% pure by weight. In some embodiments, Form H obtained according to Example 9 is at least about 99% pure by weight.

In some embodiments, Form H is at least about 95% pure by weight, and Form H comprises no more than about 5% of an impurity by weight of Form H. In some embodiments, Form H is about 95.0% to 100% pure by weight, and Form H comprises 0% to about 5% of an impurity by weight of Form H. In some embodiments, Form H is about 98% to 100% pure by weight, and Form H comprises 0% to about 2% of an impurity by weight of Form H. In some embodiments, Form H is about 98%, about 98.5%, about 99%, about 99.5%, or 100% pure by weight, and Form H comprises about 2%, about 1.5%, about 1%, about 0.5%, or 0%, respectively, of an impurity by weight of Form H. In some embodiments, Form H is about 99.5%, about 99.9%, or about 99.95% pure by weight, and Form H comprises about 0.5%, about 0.1%, or about 0.05%, respectively, of an impurity by weight of Form H. In some embodiments, the purity or the impurity are determined by high-performance liquid chromatography (HPLC).

In some embodiments, Form H comprises less than 5% of each of other forms of Compound I selected from an amorphous form, Form A, Form B, Form C, Form D, Form E, Form F, Form G, Form I, Form J, Form K, or Form L. In some embodiments, Form H comprises less than 3% of each of other forms of Compound I selected from an amorphous form, Form A, Form B, Form C, Form D, Form E, Form F, Form G, Form I, Form J, Form K, or Form L. In some embodiments, Form H comprises less than 2% of each of other forms of Compound I selected from an amorphous form, Form A, Form B, Form C, Form D, Form E, Form F, Form G, Form I, Form J, Form K, or Form L. In some embodiments, Form H comprises less than 1% of each of other forms of Compound I selected from an amorphous form, Form A, Form B, Form C, Form D, Form E, Form F, Form G, Form I, Form J, Form K, or Form L. In some embodiments, Form H is purified. In some embodiments, Form H is isolated. In some embodiments, Form H is purified and isolated.

### Form I of Compound I

In one embodiment, the present disclosure relates to a Form I of Compound I or a pharmaceutically acceptable salt, solvate, or solvate salt thereof. In one embodiment, Form I is crystalline.

In some embodiments, Form I exhibits an X-ray powder diffraction (XRPD) pattern comprising a peak at about 2.5±0.2, about 2.8±0.2, and about 8.4±0.2 degrees two-theta. In some embodiments, Form I exhibits an XRPD pattern further comprising a peak at about 5.0±0.2, about 7.4±0.2, and/or about 16.9±0.2 degrees two-theta. In some embodiments, Form I exhibits an XRPD pattern further comprising a peak at about 12.1±0.2, about 14.1±0.2, and/or about 19.7±0.2 degrees two-theta.

In some embodiments, Form I exhibits an XRPD pattern comprising 1 peak, 2 peaks, 3 peaks, 4 peaks, 5 peaks, 6 peaks, 7 peaks, 8 peaks, 9 peaks, or 10 peaks of Table I. In some embodiments, Form I exhibits an XRPD pattern comprising 1 peak, 2 peaks, 3 peaks, 4 peaks, 5 peaks, 6 peaks, 7 peaks, 8 peaks, 9 peaks, or 10 peaks of the following peaks: about 2.5±0.2, about 2.8±0.2, about 5.0±0.2, about 7.4±0.2, about 8.4±0.2, about 12.1±0.2, about 14.1±0.2, about 16.9±0.2, about 19.7±0.2, and about 22.6±0.2 degrees two-theta.

In some embodiments, Form I exhibits an XRPD pattern comprising a peak from Table I having an intensity (I %) of greater than 30%. In some embodiments, Form I exhibits an XRPD pattern comprising a peak from Table I having an intensity (I %) of greater than 5%. In some embodiments, Form I exhibits an XRPD pattern comprising a peak from Table I having an intensity (I %) of greater than 4%. In some embodiments, Form I exhibits an XRPD pattern comprising a peak from Table I having an intensity (I %) of greater than 3%. In some embodiments, Form I exhibits an XRPD pattern comprising a peak from Table I having an intensity (I %) of greater than 0.5%.

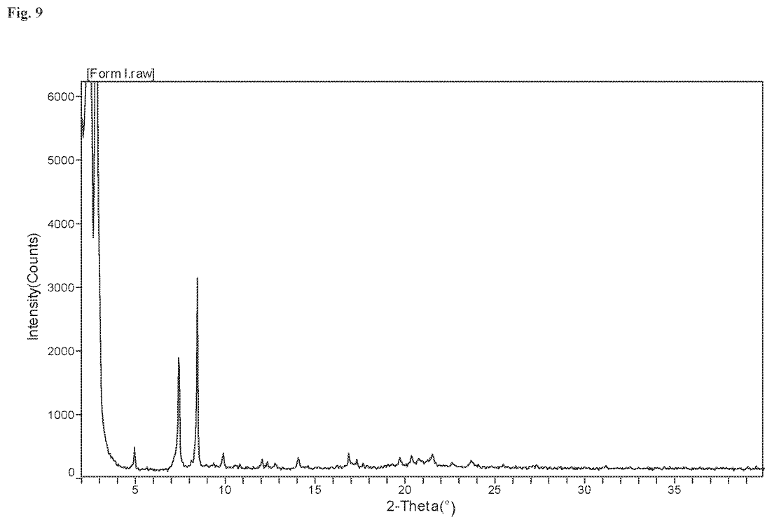

In some embodiments, Form I exhibits an XRPD pattern that is substantially the same as that depicted in FIG. 9.

TABLE I
XRPD Table of Form I of Compound I

| # | 2-Theta | d(A) | BG | Height | 1% |
|---|---|---|---|---|---|
| 1 | 2.477 | 35.6379 | 392 | 16315 | 36.3 |
| 2 | 2.844 | 31.0382 | 234 | 44898 | 100.0 |
| 3 | 4.949 | 17.8421 | 139 | 330 | 0.7 |
| 4 | 7.414 | 11.9146 | 154 | 1731 | 3.9 |
| 5 | 8.443 | 10.4643 | 175 | 2981 | 6.6 |
| 6 | 12.046 | 7.3413 | 142 | 140 | 0.3 |
| 7 | 12.781 | 6.9205 | 143 | 75 | 0.2 |
| 8 | 14.069 | 6.2899 | 139 | 168 | 0.4 |
| 9 | 16.884 | 5.2470 | 164 | 216 | 0.5 |
| 10 | 17.302 | 5.1209 | 154 | 124 | 0.3 |
| 11 | 19.725 | 4.4970 | 189 | 121 | 0.3 |
| 12 | 20.773 | 4.2726 | 198 | 93 | 0.2 |
| 13 | 21.520 | 4.1258 | 208 | 156 | 0.3 |
| 14 | 22.638 | 3.9246 | 171 | 59 | 0.1 |
| 15 | 23.681 | 3.7541 | 159 | 101 | 0.2 |
| 16 | 27.326 | 3.2610 | 147 | 44 | 0.1 |
| 17 | 31.180 | 2.8662 | 135 | 45 | 0.1 |

In some embodiments, Form I is purified. In some embodiments, Form I is isolated. In some embodiments, Form I is purified and isolated.

In some embodiments, Form I is at least about 90% pure by weight. In some embodiments, Form I is at least about 95% pure by weight. In some embodiments, Form I is at least about 95% pure by weight. In some embodiments, Form I is at least about 99% pure by weight. In some embodiments, Form I is at least about 90%, about 91%, about 92%, about 93%, about 94%, about 95%, about 96%, about 97%, about 98%, or about 99% pure by weight.

In some embodiments, Form I obtained according to Example 10 is at least about 90% pure by weight. In some embodiments, Form I obtained according to Example 10 is at least about 95% pure by weight. In some embodiments, Form I obtained according to Example 10 is at least about 95% pure by weight. In some embodiments, Form I obtained according to Example 10 is at least about 99% pure by weight.

In some embodiments, Form I is at least about 95% pure by weight, and Form I comprises no more than about 5% of an impurity by weight of Form I. In some embodiments, Form I is about 95.0% to 100% pure by weight, and Form I comprises 0% to about 5% of an impurity by weight of Form I. In some embodiments, Form I is about 98% to 100% pure by weight, and Form I comprises 0% to about 2% of an impurity by weight of Form I. In some embodiments, Form I is about 98%, about 98.5%, about 99%, about 99.5%, or 100% pure by weight, and Form I comprises about 2%, about 1.5%, about 1%, about 0.5%, or 0%, respectively, of an impurity by weight of Form I. In some embodiments, Form I is about 99.5%, about 99.9%, or about 99.95% pure by weight, and Form I comprises about 0.5%, about 0.1%, or about 0.05%, respectively, of an impurity by weight of Form I. In some embodiments, the purity or the impurity are determined by high-performance liquid chromatography (HPLC).

In some embodiments, Form I comprises less than 5% of each of other forms of Compound I selected from an amorphous form, Form A, Form B, Form C, Form D, Form E, Form F, Form G, Form H, Form J, Form K, or Form L. In some embodiments, Form I comprises less than 3% of each of other forms of Compound I selected from an amorphous form, Form A, Form B, Form C, Form D, Form E, Form F, Form G, Form H, Form J, Form K, or Form L. In some embodiments, Form I comprises less than 2% of each of other forms of Compound I selected from an amorphous form, Form A, Form B, Form C, Form D, Form E, Form F, Form G, Form H, Form J, Form K, or Form L. In some embodiments, Form I comprises less than 1% of each of other forms of Compound I selected from an amorphous form, Form A, Form B, Form C, Form D, Form E, Form F, Form G, Form H, Form J, Form K, or Form L. In some embodiments, Form I is purified. In some embodiments, Form I is isolated. In some embodiments, Form 1 is purified and isolated.

In some embodiments, Form I comprises Form F in an amount ranging from about 1% to about 90% by weight, including all subranges and values therebetween. In some embodiments, Form I comprises Form F in an amount ranging from about 5% to about 80% by weight, including all subranges and values therebetween. In some embodiments, Form I comprises Form F in an amount ranging from about 5% to about 50% by weight, including all subranges and values therebetween.

### Form J of Compound I

In one embodiment, the present disclosure relates to a Form J of Compound I or a pharmaceutically acceptable salt, solvate, or solvate salt thereof. In one embodiment, Form J is crystalline.

In some embodiments, Form J exhibits an X-ray powder diffraction (XRPD) pattern comprising a peak at about 2.8±0.2, about 8.2±0.2, and about 19.2±0.2 degrees two-theta. In some embodiments, Form J exhibits an XRPD pattern further comprising a peak at about 11.0±0.2, about 11.3±0.2, and/or about 16.4±0.2 degrees two-theta. In some embodiments, Form J exhibits an XRPD pattern further comprising a peak at about 5.5±0.2, and/or about 9.9±0.2 degrees two-theta.

In some embodiments, Form J exhibits an XRPD pattern comprising 1 peak, 2 peaks, 3 peaks, 4 peaks, 5 peaks, 6 peaks, 7 peaks, 8 peaks, 9 peaks, or 10 peaks of Table J. In some embodiments, Form J exhibits an XRPD pattern comprising 1 peak, 2 peaks, 3 peaks, 4 peaks, 5 peaks, 6 peaks, 7 peaks, or 8 peaks of the following peaks: about 2.8±0.2, about 5.5±0.2, about 8.2±0.2, about 9.9±0.2, about 11.0±0.2, about 11.3±0.2, about 16.4±0.2, and about 19.2±0.2 degrees two-theta.

In some embodiments, Form J exhibits an XRPD pattern comprising a peak from Table J having an intensity (I %) of greater than 10%. In some embodiments, Form J exhibits an XRPD pattern comprising a peak from Table J having an intensity (I %) of greater than 1%. In some embodiments, Form J exhibits an XRPD pattern comprising a peak from Table J having an intensity (I %) of greater than 0.5%. In some embodiments, Form J exhibits an XRPD pattern comprising a peak from Table J having an intensity (I %) of greater than 0.2%. In some embodiments, Form J exhibits an XRPD pattern comprising a peak from Table J having an intensity (I %) of greater than 0.1%.

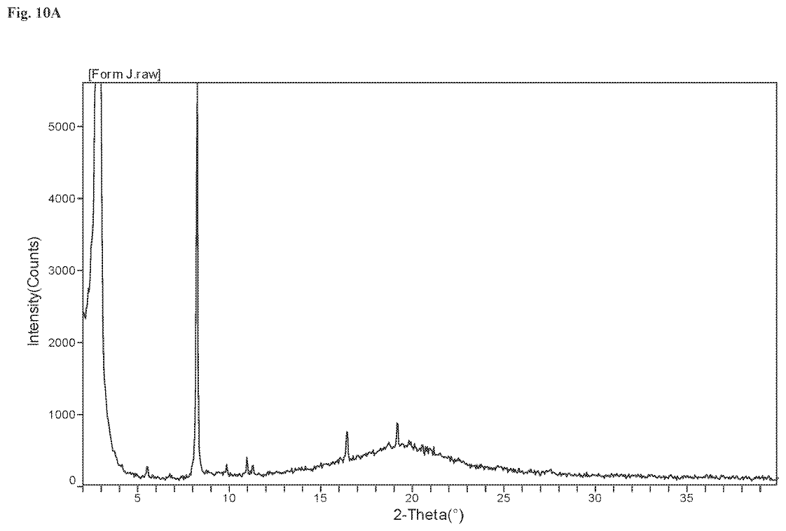

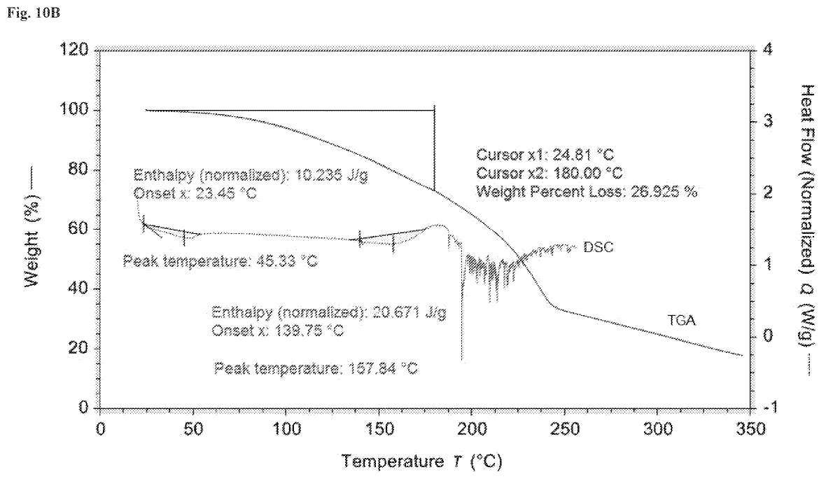

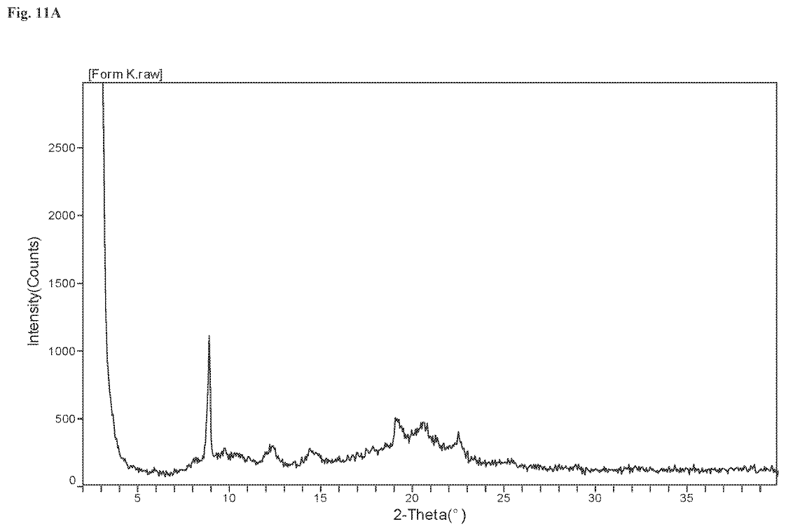

In some embodiments, Form J exhibits an XRPD pattern that is substantially the same as that depicted in FIG. 10A.

TABLE J
XRPD Table of Form J of Compound I

| # | 2-Theta | d(A) | BG | Height | 1% |
|---|---|---|---|---|---|
| 1 | 2.828 | 31.2128 | 496 | 101296 | 100.0 |
| 2 | 5.513 | 16.0182 | 265 | 144 | 0.1 |
| 3 | 8.244 | 10.7157 | 282 | 10802 | 10.7 |
| 4 | 9.851 | 8.9712 | 307 | 135 | 0.1 |
| 5 | 10.965 | 8.0622 | 298 | 247 | 0.2 |
| 6 | 11.293 | 7.8285 | 296 | 135 | 0.1 |
| 7 | 16.432 | 5.3901 | 564 | 338 | 0.3 |
| 8 | 19.183 | 4.6228 | 664 | 358 | 0.4 |

In some embodiments, Form J has a melting range from about 154 °C to about 167 °C.

In some embodiments, Form J exhibits a differential scanning calorimetry (DSC) thermogram comprising an endothermic peak having a peak maximum of from about 90 °C to about 100 °C. In some embodiments, Form J exhibits a DSC thermogram comprising an endothermic peak having a peak maximum of from about 92 °C to about 100 °C. In some embodiments, Form J exhibits a DSC thermogram comprising an endothermic peak having a peak maximum of from about 94 °C to about 98 °C. In some embodiments, Form J exhibits a DSC thermogram comprising an endothermic peak having a peak maximum of about 96 °C. In some embodiments, Form J exhibits a DSC thermogram comprising an endothermic peak that onsets at about 66 °C.

In some embodiments, Form J exhibits a differential scanning calorimetry (DSC) thermogram comprising an endothermic peak having a peak maximum of from about 148 °C to about 155 °C. In some embodiments, Form J exhibits a DSC thermogram comprising an endothermic peak having a peak maximum of from about 149 °C to about 153 °C. In some embodiments, Form J exhibits a DSC thermogram comprising an endothermic peak having a peak maximum of about 151 °C. In some embodiments, Form J exhibits a DSC thermogram comprising an endothermic peak that onsets at about 151 °C.

In some embodiments, Form J exhibits weight percent loss of about 3.0% by thermogravimetric analysis (TGA). In some embodiments, Form J exhibits weight percent loss of about 3.0% by TGA from room temperature to about 120 °C.

In some embodiments, Form J exhibits a DSC thermogram that is substantially the same as that depicted in FIG. 10B. In some embodiments, Form J exhibits a TGA thermogram that is substantially the same as that depicted in FIG. 10B.

In some embodiments, Form J comprises about 2.0 molar equivalent of NMP (27.24% by weight) by ¹H NMR.

In some embodiments, Form J is purified. In some embodiments, Form J is isolated. In some embodiments, Form J is purified and isolated.

In some embodiments, Form J is at least about 90% pure by weight. In some embodiments, Form J is at least about 95% pure by weight. In some embodiments, Form J is at least about 95% pure by weight. In some embodiments, Form J is at least about 99% pure by weight. In some embodiments, Form J is at least about 90%, about 91%, about 92%, about 93%, about 94%, about 95%, about 96%, about 97%, about 98%, or about 99% pure by weight.

In some embodiments, Form J obtained according to Example 11 is at least about 90% pure by weight. In some embodiments, Form J obtained according to Example 11 is at least about 95% pure by weight. In some embodiments, Form J obtained according to Example 11 is at least about 95% pure by weight. In some embodiments, Form J obtained according to Example 11 is at least about 99% pure by weight.

In some embodiments, Form J is at least about 95% pure by weight, and Form J comprises no more than about 5% of an impurity by weight of Form J. In some embodiments, Form J is about 95.0% to 100% pure by weight, and Form J comprises 0% to about 5% of an impurity by weight of Form J. In some embodiments, Form J is about 98% to 100% pure by weight, and Form J comprises 0% to about 2% of an impurity by weight of Form J. In some embodiments, Form J is about 98%, about 98.5%, about 99%, about 99.5%, or 100% pure by weight, and Form J comprises about 2%, about 1.5%, about 1%, about 0.5%, or 0%, respectively, of an impurity by weight of Form J. In some embodiments, Form J is about 99.5%, about 99.9%, or about 99.95% pure by weight, and Form J comprises about 0.5%, about 0.1%, or about 0.05%, respectively, of an impurity by weight of Form J. In some embodiments, the purity or the impurity are determined by high-performance liquid chromatography (HPLC).

In some embodiments, Form J comprises less than 5% of each of other forms of Compound I selected from an amorphous form, Form A, Form B, Form C, Form D, Form E. Form F, Form G, Form H, Form I, Form K, or Form L. In some embodiments, Form J comprises less than 3% of each of other forms of Compound I selected from an amorphous form, Form A, Form B, Form C, Form D, Form E, Form F, Form G, Form H, Form I, Form K, or Form L. In some embodiments, Form J comprises less than 2% of each of other forms of Compound I selected from an amorphous form, Form A, Form B, Form C, Form D, Form E, Form F, Form G, Form H, Form I, Form K, or Form L. In some embodiments, Form J comprises less than 1% of each of other forms of Compound I selected from an amorphous form, Form A, Form B. Form C, Form D, Form E, Form F, Form G, Form H, Form I, Form K, or Form L. In some embodiments, Form J is purified. In some embodiments, Form J is isolated. In some embodiments, Form J is purified and isolated.

### Form K of Compound I

In one embodiment, the present disclosure relates to a Form K of Compound I or a pharmaceutically acceptable salt, solvate, or solvate salt thereof. In one embodiment, Form K is crystalline.

In some embodiments, Form K exhibits an X-ray powder diffraction (XRPD) pattern comprising a peak at about 3.0±0.2, about 8.9±0.2, and about 19.1±0.2 degrees two-theta. In some embodiments, Form K exhibits an XRPD pattern further comprising a peak at about 22.5±0.2 degrees two-theta.

In some embodiments, Form K exhibits an XRPD pattern comprising 1 peak, 2 peaks, 3 peaks, 4 peaks, 5 peaks, 6 peaks, 7 peaks, 8 peaks, 9 peaks, or 10 peaks of Table K. In some embodiments, Form K exhibits an XRPD pattern comprising 1 peak, 2 peaks, 3 peaks, or 4 peaks, of the following peaks: about 3.0±0.2, about 8.9±0.2, about 19.1±0.2, and about 22.5±0.2 degrees two-theta.

In some embodiments, Form K exhibits an XRPD pattern comprising a peak from Table K having an intensity (I %) of greater than 50%. In some embodiments, Form K exhibits an XRPD pattern comprising a peak from Table K having an intensity (I %) of greater than 3%. In some embodiments, Form K exhibits an XRPD pattern comprising a peak from Table K having an intensity (I %) of greater than 2%. In some embodiments, Form K exhibits an XRPD pattern comprising a peak from Table K having an intensity (I %) of greater than 0.5%.

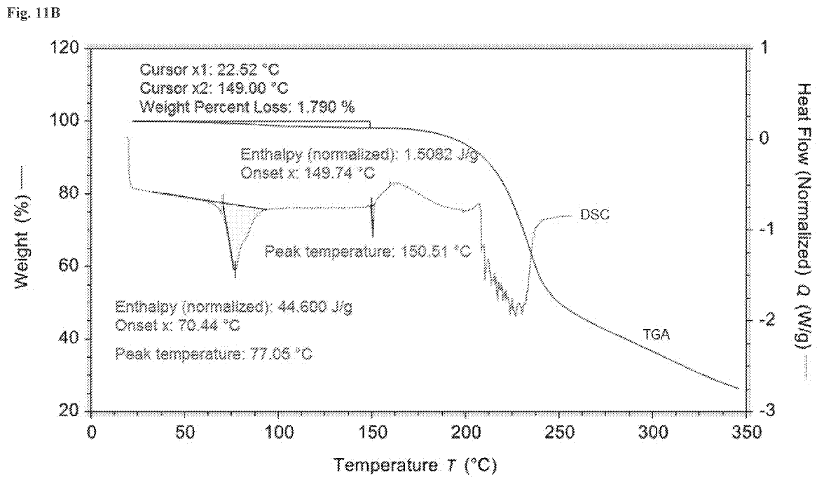

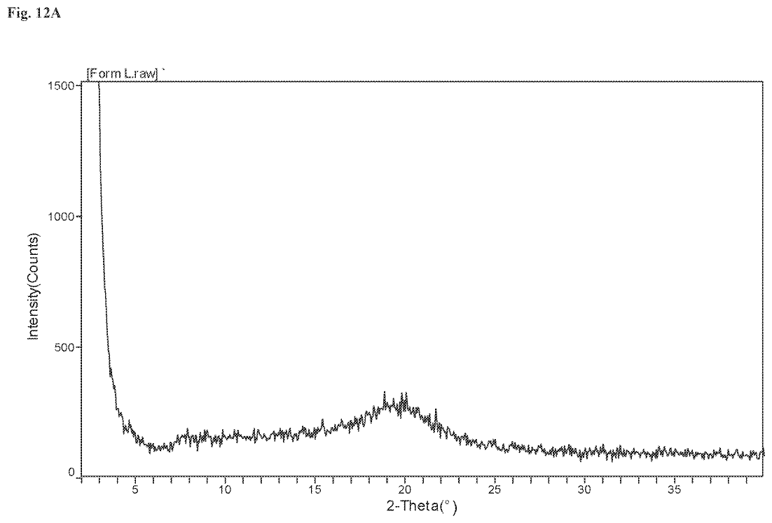

In some embodiments, Form K exhibits an XRPD pattern that is substantially the same as that depicted in FIG. 11A.

TABLE K
XRPD Table of Form K of Compound I

| # | 2-Theta | d(A) | BG | Height | 1% |
|---|---|---|---|---|---|
| 1 | 2.981 | 29.6173 | 308 | 23846 | 100.0 |
| 2 | 8.898 | 9.9301 | 267 | 918 | 3.8 |
| 3 | 19.097 | 4.6435 | 391 | 187 | 0.8 |
| 4 | 22.527 | 3.9436 | 315 | 157 | 0.7 |

In some embodiments, Form K has a melting range from about 154 °C to about 167 °C.

In some embodiments, Form K exhibits a differential scanning calorimetry (DSC) thermogram comprising an endothermic peak having a peak maximum of from about 90 °C to about 100 °C. In some embodiments, Form K exhibits a DSC thermogram comprising an endothermic peak having a peak maximum of from about 92 °C to about 100 °C. In some embodiments, Form K exhibits a DSC thermogram comprising an endothermic peak having a peak maximum of from about 94 °C to about 98 °C. In some embodiments, Form K exhibits a DSC thermogram comprising an endothermic peak having a peak maximum of about 96 °C. In some embodiments, Form K exhibits a DSC thermogram comprising an endothermic peak that onsets at about 66 °C.

In some embodiments, Form K exhibits a differential scanning calorimetry (DSC) thermogram comprising an endothermic peak having a peak maximum of from about 148 °C to about 155 °C. In some embodiments, Form K exhibits a DSC thermogram comprising an endothermic peak having a peak maximum of from about 149 °C to about 153 °C. In some embodiments, Form K exhibits a DSC thermogram comprising an endothermic peak having a peak maximum of about 151 °C. In some embodiments, Form K exhibits a DSC thermogram comprising an endothermic peak that onsets at about 151 °C.

In some embodiments, Form K exhibits weight percent loss of about 3.0% by thermogravimetric analysis (TGA). In some embodiments, Form K exhibits weight percent loss of about 3.0% by TGA from room temperature to about 120 °C.

In some embodiments, Form K exhibits a DSC thermogram that is substantially the same as that depicted in FIG. 10B. In some embodiments, Form K exhibits a TGA thermogram that is substantially the same as that depicted in FIG. 10B.

In some embodiments, Form K comprises about 2.0 molar equivalent of NMP (27.24% by weight) by ¹H NMR.

In some embodiments, Form K is purified. In some embodiments, Form K is isolated. In some embodiments, Form K is purified and isolated.

In some embodiments, Form K is at least about 90% pure by weight. In some embodiments, Form K is at least about 95% pure by weight. In some embodiments, Form K is at least about 95% pure by weight. In some embodiments, Form K is at least about 99% pure by weight. In some embodiments, Form K is at least about 90%, about 91%, about 92%, about 93%, about 94%, about 95%, about 96%, about 97%, about 98%, or about 99% pure by weight.

In some embodiments, Form K obtained according to Example 12 is at least about 90% pure by weight. In some embodiments, Form K obtained according to Example 12 is at least about 95% pure by weight. In some embodiments, Form K obtained according to Example 12 is at least about 95% pure by weight. In some embodiments, Form K obtained according to Example 12 is at least about 99% pure by weight.

In some embodiments, Form K is at least about 95% pure by weight, and Form K comprises no more than about 5% of an impurity by weight of Form K. In some embodiments, Form K is about 95.0% to 100% pure by weight, and Form K comprises 0% to about 5% of an impurity by weight of Form K. In some embodiments, Form K is about 98% to 100% pure by weight, and Form K comprises 0% to about 2% of an impurity by weight of Form K. In some embodiments, Form K is about 98%, about 98.5%, about 99%, about 99.5%, or 100% pure by weight, and Form K comprises about 2%, about 1.5%, about 1%, about 0.5%, or 0%, respectively, of an impurity by weight of Form K. In some embodiments, Form K is about 99.5%, about 99.9%, or about 99.95% pure by weight, and Form K comprises about 0.5%, about 0.1%, or about 0.05%, respectively, of an impurity by weight of Form K. In some embodiments, the purity or the impurity are determined by high-performance liquid chromatography (HPLC).

In some embodiments, Form K comprises less than 5% of each of other forms of Compound I selected from an amorphous form, Form A, Form B, Form C, Form D, Form E, Form F, Form G, Form H, Form I, Form J, or Form L. In some embodiments, Form K comprises less than 3% of each of other forms of Compound I selected from an amorphous form, Form A, Form B, Form C, Form D, Form E, Form F, Form G, Form H, Form I, Form J, or Form L. In some embodiments, Form K comprises less than 2% of each of other forms of Compound I selected from an amorphous form, Form A, Form B, Form C, Form D, Form E, Form F, Form G, Form H, Form I, Form J, or Form L. In some embodiments, Form K comprises less than 1% of each of other forms of Compound I selected from an amorphous form, Form A, Form B, Form C, Form D, Form E, Form F, Form G, Form H, Form I, Form J, or Form L. In some embodiments, Form K is purified. In some embodiments, Form K is isolated. In some embodiments, Form K is purified and isolated.

### Form L of Compound I

In one embodiment, the present disclosure relates to a Form L of Compound I or a pharmaceutically acceptable salt, solvate, or solvate salt thereof. In some embodiments, Form L is an anhydrate of Compound 1. In one embodiment, Form L is crystalline.

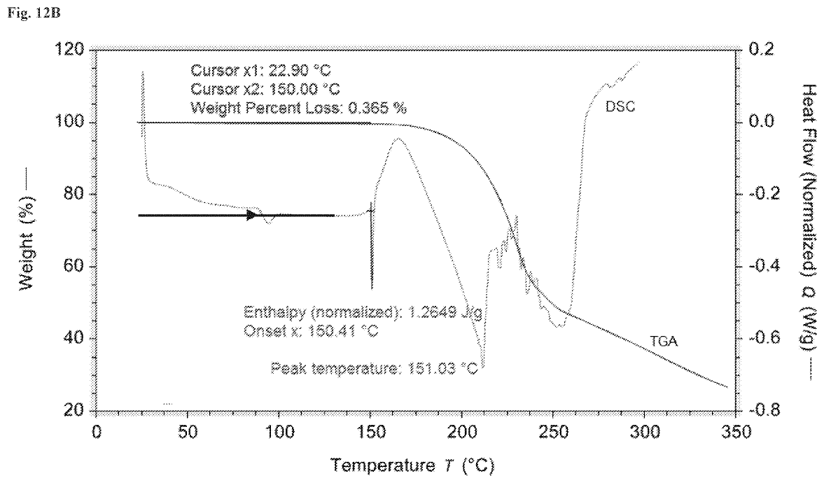

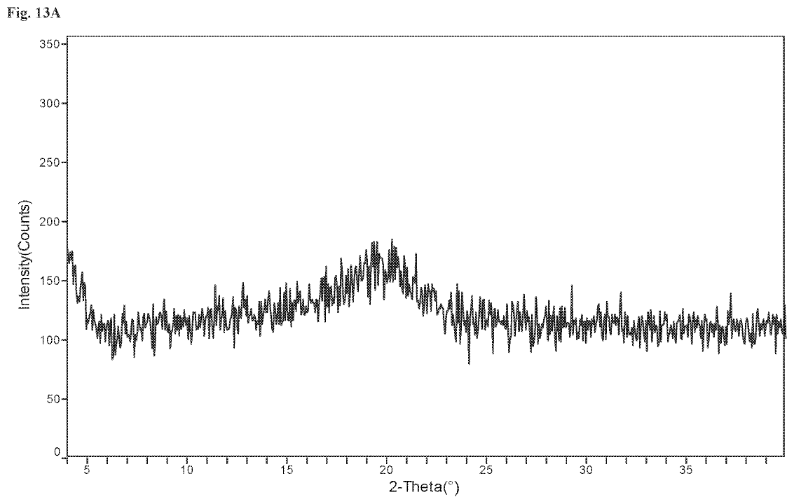

In some embodiments, Form L exhibits an XRPD pattern that is substantially the same as that depicted in FIG. 12A.

In some embodiments, Form L has a melting range from about 150 °C to about 162 °C.

In some embodiments, Form L exhibits a differential scanning calorimetry (DSC) thermogram comprising an endothermic peak having a peak maximum of from about 148 °C to about 155 °C. In some embodiments, Form L exhibits a DSC thermogram comprising an endothermic peak having a peak maximum of from about 149 °C to about 153 °C. In some embodiments, Form L exhibits a DSC thermogram comprising an endothermic peak having a peak maximum of about 151 °C. In some embodiments, Form L exhibits a DSC thermogram comprising an endothermic peak that onsets at about 150 °C.

In some embodiments, Form L exhibits weight percent loss of about 0.4% by thermogravimetric analysis (TGA). In some embodiments, Form L exhibits weight percent loss of about 0.4% by TGA from room temperature to about 150 °C.

In some embodiments, Form L exhibits a DSC thermogram that is substantially the same as that depicted in FIG. 12B. In some embodiments, Form L exhibits a TGA thermogram that is substantially the same as that depicted in FIG. 12B.

In some embodiments, Form L is purified. In some embodiments, Form L is isolated. In some embodiments, Form L is purified and isolated.

In some embodiments, Form L is at least about 90% pure by weight. In some embodiments, Form L is at least about 95% pure by weight. In some embodiments, Form L is at least about 95% pure by weight. In some embodiments, Form L is at least about 99% pure by weight. In some embodiments, Form L is at least about 90%, about 91%, about 92%, about 93%, about 94%, about 95%, about 96%, about 97%, about 98%, or about 99% pure by weight.

In some embodiments, Form L obtained according to Example 13 is at least about 90% pure by weight. In some embodiments, Form L obtained according to Example 13 is at least about 95% pure by weight. In some embodiments, Form L obtained according to Example 13 is at least about 95% pure by weight. In some embodiments, Form L obtained according to Example 13 is at least about 99% pure by weight.

In some embodiments, Form L is at least about 95% pure by weight, and Form L comprises no more than about 5% of an impurity by weight of Form L. In some embodiments. Form L is about 95.0% to 100% pure by weight, and Form L comprises 0% to about 5% of an impurity by weight of Form L. In some embodiments, Form L is about 98% to 100% pure by weight, and Form L comprises 0% to about 2% of an impurity by weight of Form L. In some embodiments, Form L is about 98%, about 98.5%, about 99%, about 99.5%, or 100% pure by weight, and Form L comprises about 2%, about 1.5%, about 1%, about 0.5%, or 0%, respectively, of an impurity by weight of Form L. In some embodiments, Form L is about 99.5%, about 99.9%, or about 99.95% pure by weight, and Form L comprises about 0.5%, about 0.1%, or about 0.05%, respectively, of an impurity by weight of Form L. In some embodiments, the purity or the impurity are determined by high-performance liquid chromatography (HPLC).

In some embodiments, Form L comprises less than 5% of each of other forms of Compound I selected from an amorphous form, Form A, Form B, Form C, Form D, Form E, Form F, Form G, Form H, Form I, Form J, or Form K. In some embodiments, Form L comprises less than 3% of each of other forms of Compound I selected from an amorphous form, Form A, Form B, Form C, Form D, Form E, Form F, Form G, Form H, Form 1, Form J, or Form K. In some embodiments, Form L comprises less than 2% of each of other forms of Compound I selected from an amorphous form, Form A, Form B, Form C, Form D, Form E, Form F, Form G, Form H, Form 1, Form J, or Form K. In some embodiments, Form L comprises less than 1% of each of other forms of Compound I selected from an amorphous form, Form A, Form B, Form C, Form D, Form E, Form F, Form G, Form H, Form I, Form J, or Form K. In some embodiments, Form L is purified. In some embodiments, Form L is isolated. In some embodiments, Form L is purified and isolated.

In some embodiments, Form L converted to Form A after placing Form L at 92.5% RH for 24 hours.

An amorphous form of Compound I

In one embodiment, the present disclosure relates to an amorphous form of Compound I or a pharmaceutically acceptable salt, solvate, or solvate salt thereof. In some embodiments, the amorphous form is anhydrous or non-solvated form of Compound I. In one embodiment, the amorphous form of Compound I is not a pharmaceutically acceptable salt.

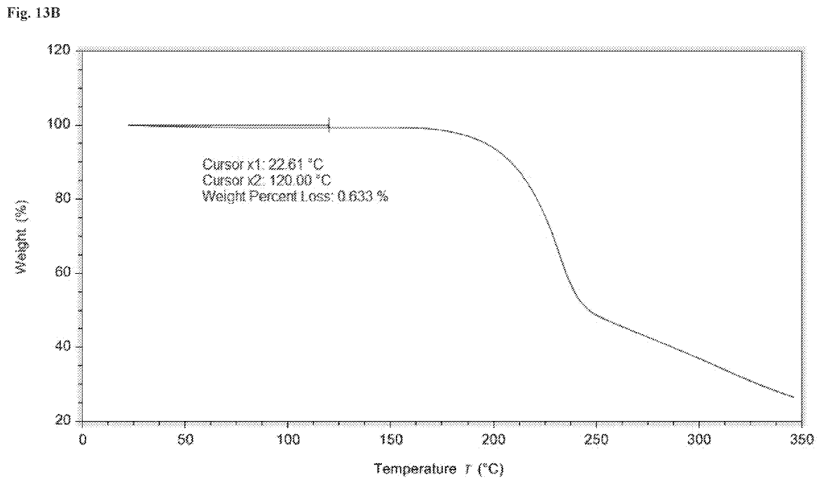

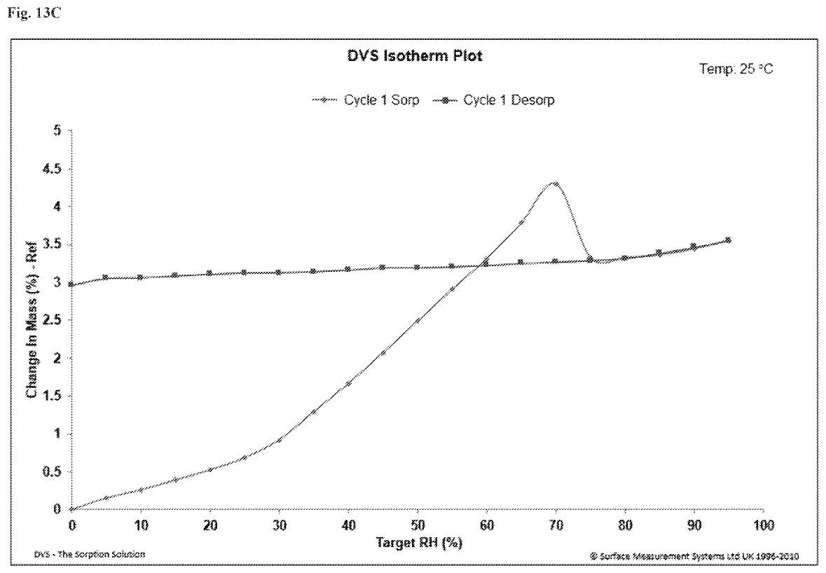

In some embodiments, the amorphous form of Compound I exhibits an XRPD pattern that is substantially the same as that depicted in FIG. 13A.

In some embodiments, the amorphous form of Compound I exhibits weight percent loss of about 0.6% by thermogravimetric analysis (TGA). In some embodiments, the amorphous form of Compound I exhibits weight percent loss of about 0.6% by TGA from room temperature to about 120 °C.

In some embodiments, the amorphous form of Compound I exhibits a TGA thermogram that is substantially the same as that depicted in FIG. 13B.

In some embodiments, the water uptake of the amorphous form of Compound I from 0% RH to 70% RH at 25 °C is about 3.5% by weight to about 5.0% by weight. In some embodiments, the water uptake of the amorphous form of Compound I from 0% RH to 70% RH at 25 °C is about 3.8% by weight to about 4.8% by weight. In some embodiments, the water uptake of the amorphous form of Compound I from 0% RH to 70% RH at 25 °C is about 4.0% by weight to about 4.6% by weight. In some embodiments, the water uptake of the amorphous form of Compound I from 0% RH to 70% RH at 25 °C is about 4.3% by weight. In some embodiments, the amorphous form of Compound I is hygroscopic. In some embodiments, water uptake is measured by dynamic vapor sorption (DVS).

In some embodiments, the amorphous form of Compound I is purified. In some embodiments, the amorphous form of Compound I is isolated. In some embodiments, the amorphous form of Compound I is purified and isolated.

In some embodiments, the amorphous form of Compound I is at least about 90% pure by weight. In some embodiments, the amorphous form of Compound I is at least about 95% pure by weight. In some embodiments, the amorphous form of Compound I is at least about 95% pure by weight. In some embodiments, the amorphous form of Compound I is at least about 99% pure by weight. In some embodiments, the amorphous form of Compound I is at least about 90%, about 91%, about 92%, about 93%, about 94%, about 95%, about 96%, about 97%, about 98%, or about 99% pure by weight.

In some embodiments, the amorphous form of Compound I is at least about 95% pure by weight, and the amorphous form of Compound I comprises no more than about 5% of an impurity by weight of the amorphous form of Compound I. In some embodiments, the amorphous form of Compound I is about 95.0% to 100% pure by weight, and the amorphous form of Compound I comprises 0% to about 5% of an impurity by weight of the amorphous form of Compound I. In some embodiments, the amorphous form of Compound I is about 98% to 100% pure by weight, and the amorphous form of Compound I comprises 0% to about 2% of an impurity by weight of the amorphous form of Compound 1. In some embodiments, the amorphous form of Compound I is about 98%, about 98.5%, about 99%, about 99.5%, or 100% pure by weight, and the amorphous form of Compound I comprises about 2%, about 1.5%, about 1%, about 0.5%, or 0%, respectively, of an impurity by weight of the amorphous form of Compound I. In some embodiments, the amorphous form of Compound I is about 99.5%, about 99.9%, or about 99.95% pure by weight, and the amorphous form of Compound I comprises about 0.5%, about 0.1%, or about 0.05%, respectively, of an impurity by weight of the amorphous form of Compound I. In some embodiments, the purity or the impurity are determined by high-performance liquid chromatography (HPLC).

In some embodiments, the amorphous form of Compound I comprises less than 5% of each of other forms of Compound I selected from Form A, Form B, Form C, Form D, Form E, Form F, Form G, Form H, Form 1, Form J, Form K, or Form L. In some embodiments, the amorphous form of Compound I comprises less than 3% of each of other forms of Compound I selected from Form A, Form B, Form C, Form D, Form E, Form F, Form G, Form H, Form 1, Form J, Form K, or Form L. In some embodiments, the amorphous form of Compound I comprises less than 2% of each of other forms of Compound I selected from Form A, Form B, Form C, Form D, Form E, Form F, Form G, Form H, Form I, Form J, Form K, or Form L. In some embodiments, the amorphous form of Compound I comprises less than 1% of each of other forms of Compound I selected from Form A, Form B, Form C, Form D, Form E, Form F, Form G, Form H, Form I, Form J, Form K, or Form L. In some embodiments, the amorphous form of Compound I is purified. In some embodiments, the amorphous form of Compound I is isolated. In some embodiments, the amorphous form of Compound I is purified and isolated.

Each of the solid forms of Compound I, or a pharmaceutically acceptable salt, solvate, or solvate salt thereof, as disclosed herein, is a “compound of the invention” (including amorphous form, Form A, Form B, Form C, Form D, Form E, Form F, Form G, Form H, Form I, Form J, Form K, or Form L).

### Pharmaceutical Compositions

The present disclosure provides pharmaceutical compositions comprising a compound of the invention and a pharmaceutically acceptable excipient or carrier. One or more excipients are added to the formulation(s) for a variety of purposes. In some embodiments, the pharmaceutical composition comprises an effective amount of the compound of the invention. In some embodiments, the pharmaceutical composition comprises a therapeutically effective amount of the compound of the invention.

In some embodiments, the pharmaceutical composition comprises a crystalline form of Compound I. In some embodiments, the pharmaceutical composition comprises Form A. In one embodiment, the pharmaceutical composition comprising a therapeutically effective amount of a Form A and a pharmaceutically acceptable excipient or carrier.

In one embodiment of the present disclosure, the pharmaceutical composition comprises Compound I or a pharmaceutically acceptable salt, solvate, or solvate salt thereof as a mixture of different forms. In one embodiment, the pharmaceutical composition comprises crystalline Form A of Compound I in about 99.9%, about 99.8%, about 99.7%, about 99.6%, about 99.5%, about 99.4%, about 99.3%, about 99.2%, about 99.1%, or about 99.0% of the total amount of Compound I or a pharmaceutically acceptable salt, solvate, or solvate salt thereof. In one embodiment, the pharmaceutical composition comprises crystalline Form A of Compound I in about 99%, 98%, 97%, 96%, 95%, 94%, 93%, 92%, 91%, or 90% of the total amount of Compound I or a pharmaceutically acceptable salt, solvate, or solvate salt thereof. In one embodiment, the pharmaceutical composition comprises crystalline Form A of Compound I in about 90%, 85%, 80%, 75%, 70%, 65%, 60%, 55%, 50%, 45%, 40%, 35%, 30%, 25%, or 20% of the total amount of Compound I or a pharmaceutically acceptable salt, solvate, or solvate salt thereof. In one embodiment, the pharmaceutical composition comprises crystalline Form A of Compound I in about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 18%, or 20% of the total amount of Compound I or a pharmaceutically acceptable salt, solvate, or solvate salt thereof.

In one embodiment, the present invention provides a pharmaceutical composition comprising a therapeutically effective amount of an amorphous form of Compound I, or a pharmaceutically acceptable salt, solvate, or solvate salt thereof, and a pharmaceutically acceptable excipient or carrier. In some embodiments, the amorphous form is anhydrous.

In one embodiment, the pharmaceutical composition comprises an amorphous form of Compound I in about 99.9%, about 99.8%, about 99.7%, about 99.6%, about 99.5%, about 99.4%, about 99.3%, about 99.2%, about 99.1%, or about 99.0% of the total amount of Compound I or a pharmaceutically acceptable salt, solvate, or solvate salt thereof. In one embodiment, the pharmaceutical composition comprises an amorphous form of Compound I in about 99%, 98%, 97%, 96%, 95%, 94%, 93%, 92%, 91%, or 90% of the total amount of Compound I or a pharmaceutically acceptable salt, solvate, or solvate salt thereof. In one embodiment, the pharmaceutical composition comprises an amorphous form of Compound 1 in about 90%, 85%, 80%, 75%, 70%, 65%, 60%, 55%, 50%, 45%, 40%, 35%, 30%, 25%, or 20% of the total amount of Compound I or a pharmaceutically acceptable salt, solvate, or solvate salt thereof. In one embodiment, the pharmaceutical composition comprises an amorphous form of Compound I in about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 18%, or 20% of the total amount of Compound 1 or a pharmaceutically acceptable salt, solvate, or solvate salt thereof.

In one embodiment of the present disclosure, the pharmaceutical composition comprises a mixture of solid forms of Compound I or a pharmaceutically acceptable salt, solvate, or solvate salt thereof. In one embodiment, the mixture comprises Form A in about 99%, 98%, 97%, 96%, 95%, 94%, 93%, 92%, 91%, or 90% and another solid form of Compound I in about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, wherein the amount represents the percentage of the total amount of Compound I or a pharmaceutically acceptable salt, solvate, or solvate salt thereof in the mixture. In one embodiment, the mixture comprises Form A in about 90%, 85%, 80%, 75%, 70%, 65%, 60%, 55%, 50%, 45%, 40%, 35%, 30%, 25%, or 20% and another solid form of Compound I in about 10%, 15%, 20%, 25%, 30%, 35%, 40%, 45%, 50%, 55%, 60%, 65%, 70%, 75%, or 80%, wherein the amount represents the percentage of the total amount of Compound I or a pharmaceutically acceptable salt, solvate, or solvate salt thereof in the mixture. In one embodiment, the mixture comprises a crystalline form of Compound I in about 99%, 98%, 97%, 96%, 95%, 94%, 93%, 92%, 91%, or 90% and an amorphous form of Compound 1 in about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, wherein the amount represents the percentage of the total amount of Compound I or a pharmaceutically acceptable salt, solvate, or solvate salt thereof in the mixture. In one embodiment, the mixture comprises a crystalline form of Compound 1 in about 90%, 85%, 80%, 75%, 70%, 65%, 60%, 55%, 50%, 45%, 40%, 35%, 30%, 25%, or 20% and an amorphous form of Compound I in about 10%, 15%, 20%, 25%, 30%, 35%, 40%, 45%, 50%, 55%, 60%, 65%, 70%, 75%, or 80%, wherein the amount represents the percentage of the total amount of Compound I or a pharmaceutically acceptable salt, solvate, or solvate salt thereof in the mixture. In one embodiment, the crystalline form of Compound I is Form A.

In one embodiment, the Compound I can be present in the pharmaceutical composition as a solvate. In one embodiment, the Compound I can be present in the pharmaceutical composition as a solvate but not a salt. In one embodiment, the Compound I can be present in the pharmaceutical composition as a hydrate. In one embodiment, the Compound I can be present in the pharmaceutical composition as a hydrate but not a salt. In one embodiment, the Compound I can be present in the pharmaceutical composition as a monohydrate. In one embodiment, the Compound I can be present in the pharmaceutical composition as a monohydrate but not a salt. In one embodiment, the Compound I can be present in the pharmaceutical composition as a pharmaceutically acceptable salt. In one embodiment, the Compound I can be present in the pharmaceutical composition as a pharmaceutical solvate salt. In one embodiment, the Compound I can be present in the pharmaceutical composition as an amorphous form.

In some embodiments, the pharmaceutical composition of the present disclosure can be formulated to be administered orally, nasally, transdermally, pulmonary, inhalationally, buccally, sublingually, intraperintoneally, subcutaneously, intramuscularly, intravenously, rectally, intrapleurally, intrathecally, intraportally, and/or parenterally. Alternatively or in addition, the pharmaceutical composition is formulated for local administration, e.g., topical. In some embodiments, the pharmaceutical composition is formulated for oral administration.

The pharmaceutically acceptable excipients are added to the composition for a variety of purposes. In one embodiment, a pharmaceutically acceptable carrier includes a pharmaceutically acceptable excipient, binder, and/or diluent. In one embodiment, suitable pharmaceutically acceptable excipients include, but are not limited to, solubilizers, emulsifiers, buffers, preservatives, sweeteners, flavoring agents, suspending agents, thickening agents, colors, viscosity regulators, stabilizers osmo-regulators, diluents, granulating agents, lubricants, binders, and disintegrating agents.

In some embodiments, the pharmaceutical composition is formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, disintegrating agents (e.g., cross-linked polyvinyl pyrrolidone, agar, or alginic acid or a salt thereof, such as sodium alginate) are added. In some embodiments, the pharmaceutical compositions comprise binders such as starches, lubricants such as talc or magnesium stearate, and/or stabilizers.

Diluents may be added to the pharmaceutical compositions as disclosed herein. Diluents increase the bulk of a solid pharmaceutical composition. Diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Binders for solid pharmaceutical compositions include, but not limited to, acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the pharmaceutical composition. Disintegrants include, but not limited to, alginic acid, carboxymethylcellulose calcium, carboxymethylcellulose sodium (e.g., ΔC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

The pharmaceutical compositions may also be dyed using any pharmaceutically acceptable colorant to improve their appearance and/or facilitate patient identification of the product and unit dosage level.

In some embodiments, the pharmaceutical composition of the present disclosure provides a sustained-release of Compound I or a pharmaceutically acceptable salt, solvate, or salt solvate thereof. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months. In some embodiments, the pharmaceutical composition provides a release of the compound of the invention over a period of at least: about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In various aspects, the amount of the one or more solid forms of Compound I, or a pharmaceutically acceptable salt, solvate, or solvate salt thereof can be administered at about 0.001 mg/kg to about 100 mg/kg body weight (e.g., about 0.01 mg/kg to about 10 mg/kg or about 0.1 mg/kg to about 5 mg/kg).

In some embodiments, an amount of the one or more solid forms of Compound 1, or a pharmaceutically acceptable salt, solvate, or solvate salt thereof, is administered to a subject irrespective of the subject's boy weight. In some embodiments, the amount of the one or more solid forms of Compound I, or a pharmaceutically acceptable salt, solvate, or solvate salt thereof administered to a subject can be about 1-5 g per day (e.g., about 1, about 2, about 3, about 4, or about 5 g per day). For example, in some embodiments, an adult subject may be administered about 2 to about 4 g per day of the one or more solid forms of Compound 1, or a pharmaceutically acceptable salt, solvate, or solvate salt thereof. The amount administered per day may be administered in multiple dosages of the one or more solid forms of Compound 1, or pharmaceutically acceptable salt, solvate, or solvate salt thereof.

The concentration or the amount of the compound of the invention in a pharmaceutically composition will vary depending on several factors, including the dosage of the solid forms of Compound I to be administered, the pharmacokinetic characteristics of the solid form(s) employed, and the route of administration. The agent may be administered in a single dose or in repeat doses. The dosage regimen utilizing the solid forms of Compound I of the present invention is selected in accordance with a variety of factors including type, species, age, weight, sex and medical condition of the patient; the severity of the condition to be treated: the route of administration; the renal and hepatic function of the patient; and the particular solid forms or salt thereof employed. Treatments may be administered daily or more frequently depending upon a number of factors, including the overall health of a patient, and the formulation and route of administration of the selected form(s). An ordinarily skilled physician or veterinarian can readily determine and prescribe the effective amount of the drug required to prevent, counter or arrest the progress of the condition.

The pharmaceutical compositions of the present disclosure may be manufactured and/or administered in single or multiple unit dose forms.

In some embodiments, any one of the pharmaceutical compositions disclosed herein can further comprise another therapeutically active agent, such as one or more therapeutically active agents useful in the treatment of sialic acid deficiency disorders such as GNE myopathy.

Each of the pharmaceutical compositions comprising the compound of the invention and a pharmaceutically acceptable carrier or excipient, as disclosed herein, is a “composition of the invention”).

### Therapeutic Use

In some embodiments, the present disclosure relates to a method for treating or preventing sialic acid deficiency, comprising administering to a subject in need thereof a therapeutically effective amount of a compound of the invention or a composition of the invention. In some embodiments, sialic acid deficiency is GNE myopathy.

In some embodiments, the present disclosure also relates to a method for treating or preventing GNE myopathy, comprising administering to a subject in need thereof a therapeutically effective amount of a compound of the invention or a composition of the invention.

In some embodiments, the present disclosure also relates to a method for delivering sialic acid to a subject in need thereof, comprising administering to the subject a therapeutically effective amount of a compound of the invention or a composition of the invention.

In some embodiments, the present disclosure also relates to a method for restoring the level of sialylation biochemistry in a subject in need thereof, comprising administering to the subject a therapeutically effective amount of a compound of the invention or a composition of the invention.

In some embodiments, the present disclosure also relates to a method for restoring the level of sialylation in muscle in a subject in need thereof, comprising administering to the subject a therapeutically effective amount of a compound of the invention or a composition of the invention.

In some embodiments of any one of the methods as disclosed herein, the solid form is Form A.

In some embodiments of any one of the methods as disclosed herein, the compound of the invention or the composition of the invention is administered in combination with another therapeutically active agent. In some embodiments, the other therapeutically active agent is administered together, sequentially, or separately with the compound of the invention or the composition of the invention. In some embodiments, the other therapeutically active agent is administered on a same or separate dosing regiment with the compound of the invention or the composition of the invention.

In some embodiments, the present disclosure relates to a means for restoring the level of sialylation biochemistry in a subject in need thereof. In some embodiments, the present disclosure relates to a means for restoring the level of sialylation in muscle in a subject in need thereof.

In some embodiments, the present disclosure relates to a means for treating or preventing sialic acid deficiency. In some embodiments, the present disclosure relates to a means for treating or preventing GNE myopathy. In some embodiments, the present disclosure relates to a means for delivering sialic acid to a subject in need thereof.

In some embodiments, the means is a solid form of Compound I or a pharmaceutically acceptable salt, solvate, or solvate salt thereof. In some embodiments, the means is a crystalline form of Compound I or a pharmaceutically acceptable salt, solvate, or solvate salt thereof. In some embodiments, the means is a Form A of Compound I monohydrate.

### EXAMPLES

The disclosure now being generally described, it will be more readily understood by reference to the following examples which are included merely for purposes of illustration of certain aspects and embodiments of the present invention, and are not intended to limit the invention

### General Procedures—Analytical Methods

X-Ray Powder Diffraction (XRPD): XRPD patterns were obtained on a Bruker D8 Advance. A CuK source (1.54056 angstrom) operating minimally at 40 kV and 40 mA scans each sample between 2 and 40 degrees 2-theta. The step size is 0.05° and scan speed is 0.5 s/step.

Differential scanning calorimetry (DSC); DSC analyses were carried out on a TA Instrument DSC unit (Model DSC 25). Samples were heated in non-hermetic aluminum pans from ambient to 300 °C at 10 °C/min with a nitrogen purge of 50 mL/mi.

Thermogravimetric Analysis (TGA): Approximately 10 mg of a sample was weighed into a platinum pan, and heat from ambient to 350 °C at the rate of 10 °C/min with N₂ purge at 60 ml/min and balance purge at 40 ml/min.

Dynamic vapor sorption (DVS) for Forms A and B: The moisture sorption profile was generated at 25 °C using a DVS moisture balance flow system (Model Advantage 1.0) with the following conditions: sample size approximately 20 mg, adsorption range 40% to 95% RH, desorption range 95% to 0% RH, adsorption range 0% to 40% RH and step interval 5%. The equilibrium criterion was <0.01% weight change in 5 minutes for a maximum of 120 minute.

Dynamic vapor sorption (DVS) for Forms C and E: The moisture sorption profile was generated at 25 °C using a DVS moisture balance flow system (Model Advantage 1.0) with the following conditions: sample size approximately 20 mg, adsorption range 0% to 95% RH, desorption range 95% to 0% RH and step interval 5%. The equilibrium criterion was <0.01% weight change in 5 minutes for a maximum of 120 minute.

Dynamic vapor sorption (DVS) for Amorphous Compound I: The moisture sorption profile was generated at 25 °C using a DVS moisture balance flow system (Model Advantage 1.0) with the following conditions: sample size approximately 10 mg, drying 25 °C for 60 minutes, adsorption range 0% to 95% RH, desorption range 95% to 0% RH and step interval 5%. The equilibrium criterion was <0.01% weight change in 5 minutes for a maximum of 120 minute.

¹H NMR: ¹H NMR spectra were collected on Bruker 400 MHz NMR Spectrometer using DMSO-d₆ as solvent.

High-performance liquid chromatography (HPLC): HPLC was used to evaluate purity of each crystalline form A-L in the following Examples, and purity in each case was found to be greater than 90%. Following instrument and parameters were generally used.

TABLE 1
HPLC parameters

| Instrument | HPLC (Agilent 1200) | |
|---|---|---|
| Column | ZORBAX SB- C8(150 mm × 4.6 mm, 3.5 um) | |
| Detection wavelength | 205 nm | |
| ELSD parameters | Evap: 40 °C | Gas: 1.2 |
| | Neb: 40 °C | Pmt: 1 |

| Mobile phase | A: 0.1% TFA in 10% ACN | | |
|---|---|---|---|
| | B: 0.1% TFA in ACN | | |
| | Time(min) | A % | B % |
| Gradient | 0 | 90 | 10 |
| | 1 | 90 | 10 |
| | 11 | 10 | 90 |
| | 15 | 10 | 90 |
| | 15.5 | 90 | 10 |
| | 22 | 90 | 10 |

| | |
|---|---|
| Flow rate | 0.9 mL/min |
| Column temperature | 50 °C |
| Injection volume | 10 μL |
| Running time | 22 min |
| Washing needle | EtOH:water = 50:50 (v/v) |
| solution | |

### Example 1. Synthesis and Characterization of Amorphous Form of Compound I

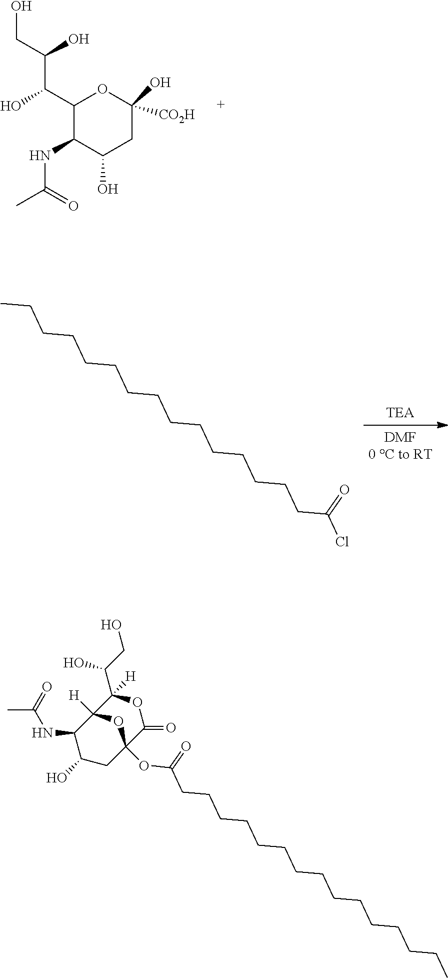 [SMILES: CC(=O)N[C@H]1C([C@H](O)[C@H](O)CO)O[C@](O)(C(=O)O)C[C@@H]1O ; CCCCCCCCCCCCCCCC(=O)Cl ; CCCCCCCCCCCCCCCC(=O)O[C@@]12C[C@H](O)[C@@H](NC(C)=O)[C@@H](O1)[C@@H]([C@H](O)CO)OC2=O]

A 5 L 3-neck round bottom flask was equipped with an overhead stirrer, addition funnel and nitrogen inlet. To a suspension of sialic acid (50 g, 161.7 mmol) in DMF (2 L) was added triethylamine (68 mL, 485.0 mmol). The solution stirred at room temperature until all solid had dissolved. The reaction was cooled to 0 °C and palmitoyl chloride (98 mL, 323.3 mmol) was added drop-wise over 20 minutes. The ice bath was removed and the reaction was allowed to warm to room temperature. After 3 hours the crude reaction mixture was filtered. The filtrate was diluted with H₂O (500 mL) and extracted with heptanes (2×1 L). Heptanes was discarded. The aqueous solution was diluted with an additional 1.5 L H₂O and extracted with EtOAc (5×1 L). The organics were concentrated down to a thick tan oil. The oil was then taken up in EtOAc (1 L) and washed with 5% LiCl (2×150 mL). The organic solution again concentrated off. The oil was taken up in 1 L MTBE and stirred vigorously. After 1 hour, the resulting precipitate was collected and dried under vacuum to yield 36 g (42%) of amorphous form of Compound I as a white powder.

ES LC-MS m/z=530 (M+H+). 1H NMR (400 MHz, CHLOROFORM-d) δ ppm 0.89 (t, J=6.74 Hz, 3H) 1.14-1.41 (m, 24H) 1.51-1.70 (m, 2H) 1.92 (br. s., 1H) 1.97-2.12 (m, 4H) 2.20 (d, J=13.28 Hz, 1H) 2.25-2.49 (m, 2H) 3.68-4.17 (m, 4H) 4.18-4.49 (m, 2H) 4.49-4.65 (m, 2H) 4.88 (br. s., 1H) 6.79 (br. s., 1H).

Amorphous form of Compound I was characterized by PLM, XRPD, TGA, DVS, and Karl-Fischer titration.

The amorphous form, evident by its XRPD (FIG. 13A), was white fine powder (after it was jet milled) and easy to agglomerate. PLM showed the material was a mixture of birefringence and non-birefringence with particles morphology, with particle size less than 10 μm. Only 0.633% weight loss from RT to 120 °C was observed on TGA curve (FIG. 13B). The assay and purity value of the amorphous form was 100.58% (HPLC, UV) and 93.41% (HPLC, UV+ELSD), respectively. The water content was about 1.35% determined by volumetric Karl Fischer titration.

The amorphous form was hygroscopic with 4.293% of water uptake from 0% RH to 70% RH at 25 °C on DVS plot, and desorption 1% water around 75% RH, and then no obvious weight change from 75% RH to 95% RH to 0% RH on DVS plot (FIG. 13C). The amorphous form converted to Form A (monohydrate) after DVS test evidenced by XRPD pattern.

### Example 2. Synthesis and Characterization of Form A of Compound I (Monohydrate)

About 500 mg of an amorphous form of Compound I was placed under 92.5% RH for 24 hours to yield crystalline Form A as a white powder (100% yield).

Form A was characterized by PLM, XRPD, DSC, TGA, DVS, and Karl-Fischer titration. The XRPD pattern (FIG. 1A) showed that Form A was crystal with high crystallinity. The PLM result showed birefringence with plates and particles morphology, and the particle size was around 2~15 μm size. The melting range of Form A was 130 °C~164 °C detected by melting point apparatus. The DSC curve (FIG. 1B) showed two endothermic peaks at 69.68 °C and 150.64 °C, respectively (see Table 2 for DSC method). The first endothermic was believed to be due to the desolvation of water, corresponding to 3.185% weight loss from RT to 150 °C on TGA curve (FIG. 1B), it's close to theoretical value (3.28%) of monohydrate. The water content was 3.49% determined by volumetric Karl Fischer titration (performed at 18 °C, 45% RH), which was consistent with TGA result.

TABLE 2
DSC Method and Results for Form A

| DSC Method | DSC Result |
|---|---|
| Ramp 10.00 °C/min to 300.00 °C | Endo. = 69.68 °C (161.45 J/g) |
| | Endo. = 150.64 °C (1.2774 J/g) |
| Equilibrate 25.00 °C | Endo. = 151.02 °C (1.5699 J/g) |
| Ramp 10.00 °C/min to 100.00 °C | |
| Ramp 10.00 °C/min to 25.00 °C | |
| Equilibrate 25.00 °C | |
| Ramp 10.00 °C/min to 300.00 °C | |

Form A was non-hygroscopic with water uptake of 0.0986% from 40% RH to 80% RH at 25 °C, no polymorph changed after DVS experiment (FIG. 1C; see Table 3 for DVS data). The DSC and TGA profile of Form A pre and post DVS were substantially the same (Table 4). The DSC profile of sample post DVS showed two endothermic peaks at 72.07 °C and 150.40 °C, respectively. 3.036% weight loss from RT to 100 °C on the TGA profile.

TABLE 3
DVS data for Form A

| | |
|---|---|
| DVS stage | Weight gain/loss |

| | | |
|---|---|---|
| Adsorption | 40% RH~80% RH | 0.0986% weight gain |
| | | (=0.3154% − 0.2168%) |
| Adsorption | 80% RH~95% RH | 0.0949% weight gain |
| | | (=0.4103% − 0.3154%) |
| Desorption | 95% RH~0% RH | 0.4094% weight loss |
| Adsorption | 0% RH~40% RH | 0.1724% weight gain |

TABLE 4
Characterization of Form A Pre- and Post-DVS

| | | | TGA |
|---|---|---|---|
| | XRPD | DSC | (weight loss) |
| Pre-DVS | Form A | Endo. = 69.68 °C (161.45 J/g) | 3.185% |
| | | Endo. = 150.64 °C (1.2774 J/g) | (RT-150 °C) |
| Post- DVS | Form A | Endo. = 72.07 °C (150.73 J/g) | 3.036% |
| | | Endo. = 150.40 °C (2.1151 J/g) | (RT-100 °C) |

### Example 3. Synthesis and Characterization of Form B of Compound I

401.33 mg of an amorphous form of Compound I was added into 4 ml glass vial, followed by the addition of 2.4 mL MTBE to obtain a suspension. Then sample was equilibrated in labquaker rotator at RT (21~23 °C). After 14 days, solids were collected and dried at 40 °C under vacuum for 5 hours to obtain 193.25 mg as a white solid (48.15% yield).

Form B was characterized by PLM, XRPD, DSC, TGA, DVS, Karl-Fischer titration, and ¹H NMR. The XRPD pattern (FIG. 2A) showed that Form B was a crystal with low crystallinity. The PLM result showed birefringence and non-birefringence with plates and particles morphology. The DSC profile (FIG. 2B) showed two endothermic peaks at 72.73 °C and 150.53 °C, respectively. The melting range of Form B was 153 °C~168 °C detected by melting point apparatus. 6.755% weight loss was observed from RT to 120 °C on the TGA profile (FIG. 2B). The water content was about 2.19% determined by volumetric Karl Fischer titration (performed at 20 °C, 39% RH). The ¹H-NMR spectra showed existence of 0.3 molar equiv. of MTBE (4.76%).

Form B was non-hygroscopic because only 0.072% weight gain from 40% RH to 80% RH on DVS plot (FIG. 2C; see Table 5 for DVS data). The DSC profile of sample post DVS showed two endothermic peaks at 71.90 °C and 150.71 °C. Post DVS sample had 3.059% weight loss from RT to 100 °C on the TGA profile. XRPD, DSC and TGA data of post DVS sample demonstrated that Form B converted to Form A after DVS experiment (Table 6).

TABLE 5
DVS data for Form B

| DVS stage | | Weight gain/loss |
|---|---|---|
| Adsorption | 40% RH~80% RH | 0.072% weight loss |
| | | (=3.725% − 3.653%) |
| Adsorption | 85% RH~95% RH | 2.912% weight loss |
| | | (=3.653% − 0.741%) |
| Desorption | 95% RH~0% RH | 0.738% weight loss |
| Adsorption | 0% RH~40% RH | 0.103% weight gain |

TABLE 6
Characterization of Form B Pre- and Post-DVS

| | | | TGA |
|---|---|---|---|
| | XRPD | DSC | (weight loss) |
| Pre-DVS | Form B | Endo. = 72.73 °C (111.30 J/g) | 6.755% |
| | | Endo. = 150.53 °C (1.3843 J/g) | (RT-120 °C) |
| Post-DVS | Form A | Endo. = 71.90 °C (174.46 J/g) | 3.059% |
| | | Endo. = 150.71 °C (1.9919 J/g) | (RT-100 °C) |

### Example 4. Synthesis and Characterization of Form C of Compound I

302.15 mg of an amorphous form of Compound I was dissolved in 1.5 mL ethanol in a 4 mL glass vial. The sample vial was placed at ambient condition (21-23 °C) and sealed with a sealing film, allowing solvents to evaporate spontaneously in the fume hood after puncture with a needle. 263.80 mg of white solid was collected (87.31% yield).

Form C was characterized by PLM, XRPD, DSC, TGA and ¹H-NMR. The XRPD pattern (FIG. 3A) showed that Form C was a crystal with high crystallinity. The PLM result showed birefringence with irregular plates morphology. The DSC profile (FIG. 3B) showed two endothermic peaks at 73.80 °C and 150.96 °C, respectively. The melting range of Form C was 153°C~168 °C detected by melting point apparatus. 5.285% weight loss was observed from RT to 150 °C on the TGA profile (FIG. 3B). The ¹H-NMR spectra showed existence of 0.5 molar equiv. of EtOH (4.17%).

Form C was slightly hygroscopic with water uptake of 0.733% from 0% RH to 80% RH at 25 °C, Form C converted to the mixture of Form A and Form C (mostly were Form A) after DVS process (FIG. 3C; see Table 7 for DVS data). The DSC profile of Form C post DVS was almost same as Form A, which showed two endothermic peaks at 69.05 °C and 150.68 °C, respectively. 3.175% weight loss from RT to 100 °C on the TGA profile (Table 8).

TABLE 7
DVS data for Form C

| DVS stage | | Weight gain/loss |
|---|---|---|
| Adsorption | 0% RH~80% RH | 0.733% weight gain |
| Adsorption | 80% RH~95% RH | 0.156% weight gain |
| Desorption | 95% RH~0% RH | 2.5% weight loss |

TABLE 8
Characterization of Form C Pre- and Post-DVS

| | | | TGA |
|---|---|---|---|
| | XRPD | DSC | (weight loss) |
| Pre-DVS | Form C | Endo. = 73.80 °C (121.74 J/g) | 5.285% |
| | | Endo. = 150.96 °C (1.2923 J/g) | (RT-150 °C) |
| Post-DVS | Form A + Form C | Endo. = 69.05 °C (153.64 J/g) | 3.175% |
| | (mostly were Form A) | Endo. = 150.68 °C (2.1991 J/g) | (RT-100 °C) |

### Example 5. Synthesis and Characterization of Form D of Compound I

About 50 mg of an amorphous form of Compound I was weighed into a 2 mL glass vial and was dissolved in 70% N-methylpyrrolidone/30% water mixture to form a slurry. Then mixture was equilibrated in labquaker rotator at about 20-21 °C for 7 days. White solids were collected by filtration (92.75% pure by HPLC. UV+ELSD).

### Example 6. Synthesis and Characterization of Form E of Compound I

300.37 mg of an amorphous form of Compound I was dissolved in 1.2 mL methanol in a 4 mL glass vial. The sample vial was placed at ambient condition (21-23 °C) and sealed with a sealing film, allowing solvents to evaporate spontaneously in the fume hood after puncture with a needle. 253.8 mg of white solid was collected (84.501% yield).

Form E was characterized by PLM, XRPD, DSC, TGA, Karl-Fischer titration, ¹H-NMR and DVS. The XRPD pattern (FIG. 5A) showed that Form E was a crystal with high crystallinity. The PLM result showed birefringence with plates and particles morphology. The DSC profile (FIG. 5B) showed three endothermic peaks at 32.99 °C, 74.18 °C and 150.95 °C, respectively. The melting range of Form E was 152 °C~163 °C detected by melting point apparatus. The water content of Form E was 2.84% determined by volumetric Karl Fischer titration. The ¹H-NMR spectra showed existence of 0.2 molar equiv. of MeOH (1.20%). 2.887% weight loss was observed from RT to 100 °C on the TGA profile (FIG. 5B), which was attributed to desolvation of water and methanol.

Form E was slightly hygroscopic with water uptake of 1.160% from 0% RH to 80% RH at 25 °C, Form E converted to the mixture of Form A and Form E (mostly were Form A) after DVS process (FIG. 5C; see Table 9 for DVS data). The DSC profile of Form E post DVS was almost same as Form A, the DSC profile showed two endothermic peaks at 66.64 °C and 150.72 °C, respectively (Table 10). 2.610% weight loss from RT to 100 °C on the TGA profile (Table 10).

TABLE 9
DVS data for Form E

| DVS stage | Process | Weight gain/loss |
|---|---|---|
| Adsorption | 0% RH~80% RH | 1.160% weight gain |
| Adsorption | 80% RH~95% RH | 0.290% weight gain |
| Desorption | 95% RH~0% RH | 0.856% weight loss |

TABLE 10
Characterization of Form E Pre- and Post-DVS

| | | | TGA |
|---|---|---|---|
| | XRPD | DSC | (weight loss) |
| Pre-DVS | Form E | Endo. = 32.99 °C (12.726 J/g) | 2.887% |
| | | Endo. = 74.18 °C (70.959 J/g) | (RT-100 °C) |
| | | Endo. = 150.95 °C (1.2832 J/g) | |
| Post-DVS | Form A + Form E | Endo. = 66.64 °C (156.39 J/g) | 2.610% |
| | (mostly Form A) | Endo. = 150.72 °C (1.4711 J/g) | (RT-100 °C) |

### Example 7. Synthesis and Characterization of Form F of Compound 1

401.02 mg of an amorphous form of Compound I was dissolved in 8 mL 50% tetrahydrofuran (THF): 50% water (v/v) to obtain a clear solution. 16 mL water was added into the solution under rapid stirring. The resulting precipitates were isolated and dried at 40 °C under vacuum for 10 hours. 385.37 mg of the dried white solid was collected (96.10% yield).

Form F was characterized by PLM, XRPD, DSC, TGA and ¹H-NMR. The XRPD pattern (FIG. 6A) showed that Form F was a crystal with medium crystallinity. The PLM result showed birefringence with plates and particles morphology. The DSC profile (FIG. 6B) showed two endothermic peaks at 48.60 °C and 149.78 °C, respectively. The melting range of Form F was 150 °C~163 °C detected by melting point apparatus. The ¹H-NMR spectra showed existence of 0.7 molar equiv. of THF (8.70%). 23.156% weight loss was observed from RT to 115 °C on the TGA profile (FIG. 6B).

### Example 8. Synthesis and Characterization of Form G of Compound I

353.91 mg of an amorphous form of Compound I was dissolved in 3 mL 2-methyltetrahydrofuran (2-MeTHF) in 4 mL glass vial at 50 °C. The solution sample was placed in −20 °C refrigerators immediately. The solids were isolated and dried at 40 °C under vacuum for 5 hours. 268.00 mg of the dried waxy-like white solid was collected (75.73% yield).

Form G was characterized by PLM, XRPD, DSC, TGA and ¹H-NMR. The XRPD pattern (FIG. 7A) showed that Form G was a crystal with medium crystallinity. The PLM result showed birefringence with plates and particles morphology. The DSC profile (FIG. 7B) showed two endothermic peaks at 77.05 °C and 150.86 °C, respectively. The melting range of Form G was 157 °C~166 °C detected by melting point apparatus. The ¹H-NMR spectra showed existence of 0.13 molar equiv. of 2-MeTHF (2.07%). 7.660% weight loss was observed from RT to 150 °C on the TGA profile (FIG. 7B).

### Example 9. Synthesis and Characterization of Form H of Compound I

293.50 mg of an amorphous form of Compound I was dissolved in 1.3 mL ethanol to obtain a clear solution. 7 mL MTBE was added into the solution under rapid stirring. The resulting solids were isolated and dried at 40 °C under vacuum for 5 hours. 124.09 mg of the dried white solid was collected (42.28% yield).

Form H was characterized by PLM, XRPD, DSC, TGA and ¹H-NMR. The XRPD pattern (FIG. 8A) showed that Form H was a crystal with medium crystallinity. The PLM result showed birefringence with plates and particles morphology. The DSC profile (FIG. 8B) showed two endothermic peaks at 66.39 °C and 150.99 °C, respectively. The melting range of Form H was 154 °C~167 °C detected by melting point apparatus. The ¹H-NMR spectra showed existence of 0.3 molar equiv. of MTBE (4.76%). 3.031% weight loss was observed from RT to 120 °C on the TGA profile (FIG. 8B), which was attributed to desolvation of MTBE.

### Example 10. Synthesis and Characterization of Form I of Compound I

About 40 mg of an amorphous form of Compound I was dissolved in 50% THF:50% water mixture in a 2 mL glass vial. The vial was placed at ambient condition (20-21 °C) and sealed with a scaling film, allowing solvents to evaporate spontaneously in the fume hood after puncture with a needle. Transparent gel with small amounts of white solids were collected.

### Example 11. Synthesis and Characterization of Form J of Compound I

599.24 mg of an amorphous form of Compound I was added into 20 ml glass vial, followed by the addition of 70% NMP/30% water (v/v) to obtain a suspension. Then suspension was stirred under room temperature (−25 °C). The suspension was kept stirring with temperature cycle (25 °C~40 °C) at about 5 °C/hour for three times. The resulting solids were isolated and dried at 40 °C under vacuum for 13 hours. 124.09 mg of the dried gel-like materials were collected (yield not determined).

Form J was characterized by PLM, XRPD, DSC, TGA and ¹H-NMR. The XRPD pattern (FIG. 10A) showed that Form J was a crystal with medium crystallinity. The PLM result showed birefringence with irregular plates morphology. The DSC profile (FIG. 10B) showed two endothermic peaks at 23.45 °C and 139.75 °C, respectively. The ¹H-NMR spectra showed existence of 2 molar equiv. of NMP (27.24%). 26.925% weight loss was observed from RT to 180 °C on the TGA profile (FIG. 10B), which was attributed to desolvation of NMP.

### Example 12. Synthesis and Characterization of Form K of Compound I

852.39 mg of an amorphous form of Compound I was added into 20 ml glass vial, followed by the addition of ethyl acetate (EA) to obtain a suspension. Then suspension was stirred under room temperature (−25 °C). The suspension was kept stirring with temperature cycle (25 °C~40 °C) at about 5 °C/hour for six times. The resulting solids were isolated and dried at 40 °C under vacuum for 10 hours. 462.81 mg of the dried waxy-like white solids were collected (54.30% yield).

Form K was characterized by PLM, XRPD, DSC, TGA and ¹H-NMR. The XRPD pattern (FIG. 11A) showed that Form K was a crystal with poor crystallinity. The PLM result showed birefringence with rods and particles morphology, and the particle size was around 2-30 μm size. The DSC profile (FIG. 11B) showed two endothermic peaks at 70.44 °C and 149.74 °C, respectively. The ¹H-NMR spectra showed existence of 0.11 molar equiv. of ethyl acetate (1.80%). 1.790% weight loss was observed from RT to 149 °C on the TGA profile (FIG. 11B), which was attributed to desolvation of ethyl acetate.

### Example 13. Synthesis and Characterization of Crystalline Form L of Compound I (Anhydrate)

About 40 mg of Compound I Form A was heated to 100 °C. The resulting transparent solids were collected (yield not determined).

Form L was characterized by PLM, XRPD, DSC, TGA and ¹H-NMR. The XRPD pattern (FIG. 12A) showed that Form L was a crystal with poor crystallinity. The PLM result showed birefringence with plates and particles morphology. The DSC profile (FIG. 12B) showed one melting point at 150.41 °C. The melting range of Form L was 150 °C~162 °C detected by melting point apparatus. The ¹H-NMR spectra showed no residual solvent. 0.365% weight loss was observed from RT to 150 °C on the TGA profile (FIG. 12B).

Form L converted to Form A after placing Form L at 92.5% RH for 24 hours.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A solid form of Compound I: 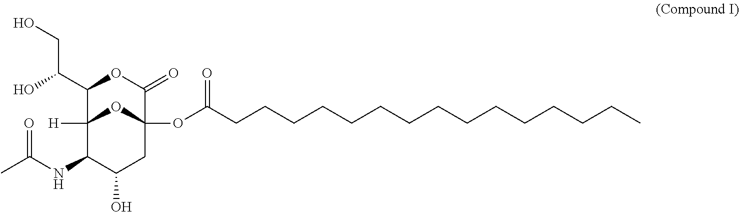 [SMILES: CCCCCCCCCCCCCCCC(=O)O[C@@]12C[C@H](O)[C@@H](NC(C)=O)[C@@H](O1)[C@@H]([C@H](O)CO)OC2=O]
or a pharmaceutically acceptable salt, solvate, or solvate salt thereof.

2. The solid form of claim 1, wherein Compound I is a hydrate.

3. The solid form of claim 1, wherein Compound I is a monohydrate.

4. The solid form of any one of claims 1-3, which exhibits an X-ray powder diffraction (XRPD) pattern comprising a peak at about 18.0±0.2, about 16.6±0.2, and about 19.5±0.2 degrees two-theta.

5. The solid form of claim 4, wherein the XRPD pattern further comprises a peak at about 3.9±0.2, about 11.5±0.2, about 15.3±0.2, and/or about 21.0±0.2 degrees two-theta.

6. The solid form of claim 4 or 5, wherein the XRPD pattern further comprises a peak at about 11.7±0.2, about 14.2±0.2, and/or about 23.6±0.2 degrees two-theta.

7. The solid form of any one of claims 1-6 that exhibits an XRPD pattern that is substantially the same as that depicted in FIG. 1A.

8. The solid form of any one of claims 1-7 that exhibits a differential scanning calorimetry (DSC) thermogram comprising an endothermic peak having a peak maximum of from about 73 °C to about 77 °C.

9. The solid form of any one of claims 1-8 that exhibits a DSC thermogram comprising an endothermic peak having a peak maximum of from about 150 °C to about 152 °C.

10. The solid form of any one of claims 1-9 that exhibits a DSC thermogram that is substantially the same as that depicted in FIG. 1B.

11. The solid form of any one of claims 1-10 that exhibits weight percent loss of about 3.2% by thermogravimetric analysis (TGA).

12. The solid form of any one of claims 1-11 that exhibits a TGA thermogram that is substantially the same as that depicted in FIG. 1B.

13. The solid form of any one of claims 1-12, wherein water uptake by the crystalline form from 40% RH to 80% RH at 25 °C is less than about 0.2% by weight.

14. The solid form of any one of claims 1-13, wherein the crystalline form is Form A.

15. The solid form of claim 1, which exhibits an X-ray powder diffraction (XRPD) pattern comprising a peak at about 2.8±0.2, about 8.4±0.2, and about 19.9±0.2 degrees two-theta.

16. The solid form of claim 15, wherein the XRPD pattern further comprises a peak at about 20.3±0.2, about 9.9±0.2, and/or about 13.9±0.2 degrees two-theta.

17. The crystalline form of claim 15 or 16, wherein the XRPD pattern further comprises a peak at about 19.1±0.2, about 9.1±0.2, and/or about 12.3±0.2 degrees two-theta.

18. The solid form of any one of claims 15-17 that exhibits an XRPD pattern that is substantially the same as that depicted in FIG. 2A.

19. The solid form of any one of claims 15-18 that exhibits a differential scanning calorimetry (DSC) thermogram comprising:
a) an endothermic peak having a peak maximum of from about 77 °C to about 82 °C; and/or
b) an endothermic peak having a peak maximum of from about 148 °C to about 152 °C.

20. The solid form of any one of claims 15-19 that exhibits weight percent loss of about 6.8% by thermogravimetric analysis (TGA).

21. The solid form of any one of claims 15-20 that exhibits:
a) a DSC thermogram that is substantially the same as that depicted in FIG. 2B; and/or
b) a TGA thermogram that is substantially the same as that depicted in FIG. 2B.

22. The solid form of any one of claims 15-21, wherein the crystalline form is Form B.

23. The solid form of claim 1, which exhibits an X-ray powder diffraction (XRPD) pattern comprising a peak at about 2.9±0.2, about 8.5±0.2, and about 17.0±0.2 degrees two-theta.

24. The solid form of claim 23, wherein the XRPD pattern further comprises a peak at about 19.9±0.2, about 14.1±0.2, and/or about 22.9±0.2 degrees two-theta.

25. The solid form of claim 23 or 24, wherein the XRPD pattern further comprises a peak at about 10.6±0.2, about 19.3±0.2, and/or about 25.6±0.2 degrees two-theta.

26. The solid form of any one of claims 23-25 that exhibits an XRPD pattern that is substantially the same as that depicted in FIG. 3A.

27. The solid form of any one of claims 23-26 that exhibits a differential scanning calorimetry (DSC) thermogram comprising:
a) an endothermic peak having a peak maximum of from about 81 °C to about 85 °C; and/or
b) an endothermic peak having a peak maximum of from about 148 °C to about 152 °C.

28. The solid form of any one of claims 23-27 that exhibits weight percent loss of about 5.3% by thermogravimetric analysis (TGA).

29. The solid form of any one of claims 23-28 that exhibits:
a) a DSC thermogram that is substantially the same as that depicted in FIG. 3B; and/or
b) a TGA thermogram that is substantially the same as that depicted in FIG. 3B.

30. The solid form of any one of claims 23-29, wherein the crystalline form is Form C.

31. The solid form of claim 1, which exhibits an X-ray powder diffraction (XRPD) pattern comprising a peak at about 2.8±0.2, about 3.9±0.2, and about 8.2±0.2 degrees two-theta.

32. The solid form of claim 31, wherein the XRPD pattern further comprises a peak at about 15.4±0.2, about 11.5±0.2, and/or about 7.7±0.2 degrees two-theta.

33. The solid form of claim 31 or 32, wherein the XRPD pattern further comprises a peak at about 19.2±0.2, about 16.4±0.2, and/or about 34.9±0.2 degrees two-theta.

34. The solid form of any one of claims 31-33 that exhibits an XRPD pattern that is substantially the same as that depicted in FIG. 4.

35. The solid form of any one of claims 31-34, wherein the crystalline form is Form D.

36. The solid form of claim 1, which exhibits an X-ray powder diffraction (XRPD) pattern comprising a peak at about 3.1±0.2, about 9.3±0.2, and about 18.6±0.2 degrees two-theta.

37. The solid form of claim 36, wherein the XRPD pattern further comprises a peak at about 19.3±0.2, about 6.2±0.2, and/or about 21.8±0.2 degrees two-theta.

38. The solid form of claim 36 or 37, wherein the XRPD pattern further comprises a peak at about 24.8±0.2, about 12.4±0.2, and/or about 15.0±0.2 degrees two-theta.

39. The solid form of any one of claims 36-38 that exhibits an XRPD pattern that is substantially the same as that depicted in FIG. 5A.

40. The solid form of any one of claims 36-39 that exhibits a differential scanning calorimetry (DSC) thermogram comprising:
a) an endothermic peak having a peak maximum of from about 34 °C to about 38 °C;
b) an endothermic peak having a peak maximum of from about 76 °C to about 80 °C; and/or
C) an endothermic peak having a peak maximum of from about 148 °C to about 152 °C.

41. The solid form of any one of claims 36-40 that exhibits weight percent loss of about 2.9% by thermogravimetric analysis (TGA).

42. The solid form of any one of claims 36-41 that exhibits:
a) a DSC thermogram that is substantially the same as that depicted in FIG. 5B; and/or
b) a TGA thermogram that is substantially the same as that depicted in FIG. 5B.

43. The solid form of any one of claims 36-42, wherein the crystalline form is Form E.

44. The solid form of claim 1, which exhibits an X-ray powder diffraction (XRPD) pattern comprising a peak at about 2.9±0.2, about 8.5±0.2, and about 20.3±0.2 degrees two-theta.

45. The solid form of claim 44, wherein the XRPD pattern further comprises a peak at about 14.1±0.2, about 19.7±0.2, and/or about 10.0±0.2 degrees two-theta.

46. The solid form of claim 44 or 45, wherein the XRPD pattern further comprises a peak at about 10.6±0.2, about 22.7±0.2, and/or about 16.9±0.2 degrees two-theta.

47. The solid form of any one of claims 44-46 that exhibits an XRPD pattern that is substantially the same as that depicted in FIG. 6A.

48. The solid form of any one of claims 44-47 that exhibits a differential scanning calorimetry (DSC) thermogram comprising:
a) an endothermic peak having a peak maximum of from about 81 °C to about 85 °C; and/or
b) an endothermic peak having a peak maximum of from about 148 °C to about 152 °C.

49. The solid form of any one of claims 44-48 that exhibits weight percent loss of about 23.2% by thermogravimetric analysis (TGA).

50. The solid form of any one of claims 44-49 that exhibits:
a) a DSC thermogram that is substantially the same as that depicted in FIG. 6B; and/or
b) a TGA thermogram that is substantially the same as that depicted in FIG. 6B.

51. The solid form of any one of claims 44-50, wherein the crystalline form is Form F.

52. The solid form of claim 1, which exhibits an X-ray powder diffraction (XRPD) pattern comprising a peak at about 2.8±0.2, about 8.3±0.2, and about 19.9±0.2 degrees two-theta.

53. The solid form of claim 52, wherein the XRPD pattern further comprises a peak at about 13.9±0.2, about 9.8±0.2, and/or about 10.5±0.2 degrees two-theta.

54. The solid form of claim 52 or 53, wherein the XRPD pattern further comprises a peak at about 11.3±0.2, about 19.3±0.2, and/or about 24.9±0.2 degrees two-theta.

55. The solid form of any one of claims 52-54 that exhibits an XRPD pattern that is substantially the same as that depicted in FIG. 7A.

56. The solid form of any one of claims 52-55 that exhibits a differential scanning calorimetry (DSC) thermogram comprising:
a) an endothermic peak having a peak maximum of from about 90 °C to about 95 °C; and/or
b) an endothermic peak having a peak maximum of from about 150 °C to about 154 °C.

57. The solid form of any one of claims 52-56 that exhibits weight percent loss of about 7.7% by thermogravimetric analysis (TGA).

58. The solid form of any one of claims 52-57 that exhibits:
a) a DSC thermogram that is substantially the same as that depicted in FIG. 7B; and/or
b) a TGA thermogram that is substantially the same as that depicted in FIG. 7B.

59. The solid form of any one of claims 52-58, wherein the crystalline form is Form G.

60. The solid form of claim 1, which exhibits an X-ray powder diffraction (XRPD) pattern comprising a peak at about 2.8±0.2, about 8.1±0.2, and about 21.7±0.2 degrees two-theta.

61. The solid form of claim 60, wherein the XRPD pattern further comprises a peak at about 10.8±0.2, about 16.3±0.2, and/or about 19.0±0.2 degrees two-theta.

62. The solid form of claim 60 or 61, wherein the XRPD pattern further comprises a peak at about 5.4±0.2, about 19.8±0.2, and/or about 22.0±0.2 degrees two-theta.

63. The solid form of any one of claims 60-62 that exhibits an XRPD pattern that is substantially the same as that depicted in FIG. 8A.

64. The solid form of any one of claims 60-63 that exhibits a differential scanning calorimetry (DSC) thermogram comprising:
a) an endothermic peak having a peak maximum of from about 94 °C to about 98 °C; and/or
b) an endothermic peak having a peak maximum of from about 149 °C to about 153 °C.

65. The solid form of any one of claims 60-64 that exhibits weight percent loss of about 3.0% by thermogravimetric analysis (TGA).

66. The solid form of any one of claims 60-65 that exhibits:
a) a DSC thermogram that is substantially the same as that depicted in FIG. 8B; and/or
b) a TGA thermogram that is substantially the same as that depicted in FIG. 8B.

67. The solid form of any one of claims 60-66, wherein the crystalline form is Form H.

68. The solid form of claim 1, which exhibits an X-ray powder diffraction (XRPD) pattern comprising a peak at about 2.5±0.2, about 2.8±0.2, and about 8.4±0.2 degrees two-theta.

69. The solid form of claim 68, wherein the XRPD pattern further comprises a peak at about 5.0±0.2, about 7.4±0.2, and/or about 16.9±0.2 degrees two-theta.

70. The solid form of claim 68 or 69, wherein the XRPD pattern further comprises a peak at about 12.1±0.2, about 14.1±0.2, and/or about 19.7±0.2 degrees two-theta.

71. The solid form of any one of claims 68-70 that exhibits an XRPD pattern that is substantially the same as that depicted in FIG. 9.

72. The solid form of any one of claims 68-71, wherein the crystalline form is Form I.

73. The solid form of claim 1, which exhibits an X-ray powder diffraction (XRPD) pattern comprising a peak at about 2.8±0.2, about 8.2±0.2, and about 19.2±0.2 degrees two-theta.

74. The solid form of claim 73, wherein the XRPD pattern further comprises a peak at about 11.0±0.2, about 11.3±0.2, and/or about 16.4±0.2 degrees two-theta.

75. The solid form of claim 73 or 74, wherein the XRPD pattern further comprises a peak at about 5.5±0.2, and/or about 9.9±0.2 degrees two-theta.

76. The solid form of any one of claims 73-75 that exhibits an XRPD pattern that is substantially the same as that depicted in FIG. 10A.

77. The solid form of any one of claims 73-76 that exhibits a differential scanning calorimetry (DSC) thermogram comprising:
a) an endothermic peak having a peak maximum of from about 40 °C to about 50 °C; and/or
b) an endothermic peak having a peak maximum of from about 150 °C to about 165 °C.

78. The solid form of any one of claims 73-77 that exhibits weight percent loss of about 27.0% by thermogravimetric analysis (TGA).

79. The solid form of any one of claims 73-78 that exhibits:
a) a DSC thermogram that is substantially the same as that depicted in FIG. 10B; and/or
b) a TGA thermogram that is substantially the same as that depicted in FIG. 10B.

80. The solid form of any one of claims 73-79, wherein the crystalline form is Form J.

81. The solid form of claim 1, which exhibits an X-ray powder diffraction (XRPD) pattern comprising a peak at about 3.0±0.2, about 8.9±0.2, and about 19.1±0.2 degrees two-theta.

82. The solid form of claim 81, wherein the XRPD pattern further comprises a peak at about 22.5±0.2 degrees two-theta.

83. The solid form of any one of claims 81-82 that exhibits an XRPD pattern that is substantially the same as that depicted in FIG. 11A.

84. The solid form of any one of claims 81-83 that exhibits a differential scanning calorimetry (DSC) thermogram comprising:
a) an endothermic peak having a peak maximum of from about 75 °C to about 80 °C; and/or
b) an endothermic peak having a peak maximum of from about 148 °C to about 153 °C.

85. The solid form of any one of claims 81-84 that exhibits weight percent loss of about 1.8% by thermogravimetric analysis (TGA).

86. The solid form of any one of claims 81-85 that exhibits:
a) a DSC thermogram that is substantially the same as that depicted in FIG. 11B; and/or
b) a TGA thermogram that is substantially the same as that depicted in FIG. 11B.

87. The solid form of any one of claims 81-86, wherein the crystalline form is Form K.

88. The solid form of claim 1, wherein Compound I is an anhydrate.

89. The solid form of claim 1 or 88 that exhibits an XRPD pattern that is substantially the same as that depicted in FIG. 12A.

90. The solid form of 88 or 89 that exhibits a differential scanning calorimetry (DSC) thermogram comprising an endothermic peak having a peak maximum of from about 148 °C to about 153 °C.

91. The solid form of any one of claims 88-90 that exhibits weight percent loss of about 0.4% by thermogravimetric analysis (TGA).

92. The solid form of any one of claims 88-91 that exhibits:
a) a DSC thermogram that is substantially the same as that depicted in FIG. 12B; and/or
b) a TGA thermogram that is substantially the same as that depicted in FIG. 12B.

93. The solid form of any one of claims 88-92, wherein the crystalline form is Form L.

94. The solid form of any one of claim 1-93, wherein the solid form is a crystalline form.

95. The solid form of any one of claim 1-94, wherein the solid form is a polymorph.

96. The solid form of claim 88, wherein the solid form is an amorphous form.

97. The solid form of claim 96, wherein Compound I is not present as a pharmaceutically acceptable salt.

98. The solid form of 96 or 97 that exhibits an XRPD pattern that is substantially the same as that depicted in FIG. 13A.

99. The solid form of any one of claims 96-98 that exhibits weight percent loss of about 0.6% by thermogravimetric analysis (TGA).

100. The solid form of any one of claims 96-99 that exhibits a TGA thermogram that is substantially the same as that depicted in FIG. 13B.

101. The solid form of any one of claims 96-100, wherein water uptake by the solid form from 0% RH to 70% RH at 25 °C is about 4.0% by weight to 4.5% by weight.

102. The solid form of any one of claims 1-101, wherein the solid form is isolated.

103. The solid form of any one of claims 1-102, wherein the solid form is purified.

104. A composition comprising a solid form of any one of claims 1-103 and a pharmaceutically acceptable carrier.

105. The composition of claim 104, wherein the solid form is Form A.

106. The composition of claim 104, wherein the solid form is an amorphous form of Compound I.

107. The composition of claim 104, wherein the composition comprises one or more solid forms selected from Form A, Form B, Form C, Form D, Form E, Form F, Form G, Form H, Form I, Form J, Form K, Form L, or an amorphous form.

108. The composition of claim 104, further comprising Form B, Form C, Form E, Form L, and/or an amorphous form.

109. The composition of any one of claims 104-108, further comprising another therapeutically active agent.

110. The composition of any one of claims 104-109, wherein the composition is formulated for an oral dosage form.

111. The composition of claim 110, wherein the oral dosage form is a solid oral dosage form.

112. The composition of claim 111, wherein the solid oral dosage form comprises at least one tablet, at least one capsule, or granules.

113. The composition of claim 110, wherein the oral dosage form is a liquid oral dosage form.

114. A method for treating or preventing sialic acid deficiency, comprising administering to a subject in need thereof a therapeutically effective amount of the solid form of any one of claims 1-103 or any one of the composition of claims 104-113.

115. A method for treating or preventing GNE myopathy, comprising administering to a subject in need thereof a therapeutically effective amount of the solid form of any one of claims 1-103 or any one of the composition of claims 104-113.
